SEQUENCE LISTING (14 sequences)

SEQ ID NO: 1 - DNA, 47 bp, Artificial Sequence
  Synthetic polynucleotide
nnnnnnnnnnnnnnnnnnnngatcatcgtcagcagtcgcgtagctag

SEQ ID NO: 2 - DNA, 24 bp, Artificial Sequence
  Synthetic polynucleotide
gctacgcgactgctgacgatgatc

SEQ ID NO: 3 - DNA, 35 bp, Artificial Sequence
  Synthetic polynucleotide
ctagctacgcgactgctgacgatgatcnnnncnnn

SEQ ID NO: 4 - DNA, 45 bp, Artificial Sequence
  Synthetic polynucleotide
nnnnnnnnnnnnngnnnngatcatcgtcagcagtcgcgtagctag

SEQ ID NO: 5 - DNA, 28 bp, Artificial Sequence
  Synthetic polynucleotide
gctacgcgactgctgacgatgatcnnnn

SEQ ID NO: 6 - DNA, 39 bp, Artificial Sequence
  Synthetic polynucleotide
ctagctacgcgactgctgacgatgctannnnnnncnnnn

SEQ ID NO: 7 - DNA, 17 bp, Artificial Sequence
  Synthetic polynucleotide
ctagctacgcgactgct

SEQ ID NO: 8 - DNA, 15 bp, Artificial Sequence
  Synthetic polynucleotide
gacgatgatcnnnnn

SEQ ID NO: 9 - DNA, 32 bp, Artificial Sequence
  Synthetic polynucleotide
ctagctacgcgactgctgacgatgatcnnnnn

SEQ ID NO: 10 - DNA, 40 bp, Artificial Sequence
  Synthetic polynucleotide
ctagctacgcgactgctgacgatcctannnnnnnnnannn

SEQ ID NO: 11 - DNA, 47 bp, Artificial Sequence
  Synthetic polynucleotide
nnnnnnnnnntnnnnnnnnngatcatcgtcagcagtcgcgtagctag

SEQ ID NO: 12 - DNA, 37 bp, Artificial Sequence
  Synthetic polynucleotide
nnnnnnnnnnnnnnnnnnnnagcagtcgcgtagctag

SEQ ID NO: 13 - DNA, 24 bp, Artificial Sequence
  Synthetic polynucleotide
ctagctacgcgactgctnnnnnnn

SEQ ID NO: 14 - DNA, 40 bp, Artificial Sequence
  Synthetic polynucleotide
ctagctacgcgactgctgacgatgatcnnnnnnnnnannn

(19) United States
(12) Patent Application Publication
(10) Pub. No.: US 2013/0296173 A1
(43) Pub. Date: Nov. 7, 2013

(54) PRE-ANCHOR WASH

(71) Applicant: Complete Genomics, Inc., Mountain View, CA (US)

(72) Inventors: Matthew Callow, San Mateo, CA (US); Linsu Chen, Cupertino, CA (US); Dennis Ballinger, Menlo Park, CA (US)

(21) Appl. No.: 13/868,000

(22) Filed: Apr. 22, 2013

Related U.S. Application Data
(60) Provisional application No. 61/637,240, filed on Apr. 23, 2012.

(51) Int. Cl. C12Q 1/68

(52) U.S. Cl. CPC C12Q 1/6874
USPC 506/2; 506/42

(57) ABSTRACT

The present invention is directed to compositions and methods for improving the discordance rate and mapping yield in nucleic acid sequencing reactions.

### CROSS-REFERENCE TO RELATED APPLICATIONS

This application claims the benefit of priority to U.S. Provisional Patent Application No. 61/637,240, filed Apr. 23, 2012, which is hereby incorporated herein by reference in its entirety for all purposes.

### BACKGROUND OF THE INVENTION

Biochemical assays performed on nucleic acid molecules, such as DNA sequencing, for example, can subject the DNA molecules to a harsh environment that affects the data resulting from such assays. For example, after multiple cycles of DNA sequencing reactions performed on DNA molecules that are arrayed on a solid substrate, there can be an increase in the discordance rate and a reduction in mapping yield.

### SUMMARY OF THE INVENTION

The present invention is directed to methods and compositions for improving discordance, mappable yield and other metrics of nucleic acid sequencing reactions. In particular, according to one embodiment, a “pre-anchor wash”—an aqueous wash solution that includes an effective amount of a weak acid or a cationic surfactant—is used. In the description of the invention that follows, this wash step is described as occurring after attachment of a nucleic acid to the surface of a solid support and before performing the sequencing reaction in each cycle or in later cycles. However, it can occur at other points in the sequencing cycle.

According to one aspect, the present invention provides methods of sequencing a target sequence of a nucleic acid molecule, the method comprising: (a) providing a surface comprising the nucleic acid molecule, the nucleic acid molecule comprising: (i) a first adaptor comprising a first anchor site; and (ii) the target sequence; (b) applying to the surface an aqueous wash solution comprising an effective amount of a member of the group consisting of an acid, a cationic surfactant, and both an acid and a cationic surfactant; (c) hybridizing an anchor to the first anchor site; (d) extending the anchor to produce an anchor extension product; (e) detecting the extension product, thereby identifying a base of the target sequence; and (f) repeating steps (b) to (e) until the sequence of the target sequence is determined. According to one embodiment, the surface comprising the nucleic acid molecule is an nucleic acid array comprising a surface and a plurality of the nucleic acid molecules attached to the surface. According to another embodiment, the nucleic acid molecule is a concatemer comprising a plurality of monomer units, each monomer unit comprising the first adaptor and the target sequence. According to another embodiment, such methods comprise applying to the surface an aqueous wash solution before hybridizing the anchor to the first anchor site, although the aqueous wash solution can be applied at other steps in the sequencing cycle.

Such methods can be used in connection with a number of sequencing technologies. According to another embodiment, such methods comprise extending the anchor by adding a nucleotide to the anchor or a product of a previous extension of the anchor (e.g., as in sequencing-by synthesis). According to another embodiment, such methods comprise extending the anchor by ligating a sequencing probe to the anchor or a product of a previous extension of the anchor. According to one embodiment, such methods are used in the context of cPAL sequencing biochemistry, including double cPAL. Accordingly, according to one embodiment, such methods comprise extending the anchor by: (i) ligating one or more extension anchors to the anchor, and (ii) ligating the sequence probe to said one or more extension anchors.

According to another embodiment, such methods comprise stripping the extension product from the nucleic acid molecule before repeating steps (b) to (e).

The pre-anchor wash reagent can comprise various weak acids and cationic surfactants, for example. According to one embodiment, the acid is citric acid. According to another embodiment, the cationic surfactant is CTAB.

According to another aspect, the aqueous wash solution comprises an amount of an acid or a cationic surfactant that is effective to reduce discordance by 5 percent or more or to increase a mappable yield by 0.5 percent or more or both compared to a suitable control.

According to another aspect, an aqueous wash solution is provided for sequencing a nucleic acid molecule attached to a surface, the wash solution comprising a member of the group consisting of an acid, a cationic surfactant, and both, wherein the wash solution is effective to detectably reduce discordance, e.g., by 5 percent or more, or to detectably increase a mappable yield, e.g., by 0.5 percent or more, or both, when compared to a suitable control.

### BRIEF DESCRIPTION OF THE DRAWINGS

FIG. 1 is a schematic illustration of one embodiment of a combinatorial probe-anchor ligation method.

FIG. 2 is a schematic illustration of one embodiment of a combinatorial probe-anchor ligation method.

FIG. 3 is a schematic illustration of one embodiment of a combinatorial probe-anchor ligation method.

FIG. 4 is a schematic illustration of one embodiment of a combinatorial probe anchor ligation method.

FIG. 5 shows results from use of a pre-anchor wash with 0.1 mM CTAB or 10 mM citric acid.

### DETAILED DESCRIPTION OF THE INVENTION

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry, 3^{rd} Ed., W. H. Freeman Pub., New York, N.Y. and Berg et al. (2002) Biochemistry, 5th Ed., W. H. Freeman Pub., New York, N.Y., all of which are herein incorporated in their entirety by reference for all purposes.

Note that as used herein and in the appended claims, the singular forms “a,” “an,” and “the” include plural referents unless the context clearly dictates otherwise. Thus, for example, reference to “a polymerase” refers to one agent or mixtures of such agents, and reference to “the method” includes reference to equivalent steps and methods known to those skilled in the art, and so forth.

Unless defined otherwise, all technical and scientific terms used herein have the same meaning as commonly understood by one of ordinary skill in the art to which this invention belongs. All publications mentioned herein are incorporated herein by reference for the purpose of describing and disclosing devices, compositions, formulations and methodologies which are described in the publication and which might be used in connection with the presently described invention.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

Although the present invention is described primarily with reference to specific embodiments, it is also envisioned that other embodiments will become apparent to those skilled in the art upon reading the present disclosure, and it is intended that such embodiments be contained within the present inventive methods.

### Overview

The present invention is directed to methods and compositions for improving discordance, mappable yield and other metrics of nucleic acid sequencing reactions. In particular, according to one embodiment, a “pre-anchor wash”—an aqueous wash solution that includes an effective amount of a weak acid or a cationic surfactant—is used in each cycle. In the description of the invention that follows, this wash step is described as occurring after attachment of a nucleic acid to the surface of a solid support and before performing the sequencing reaction in each cycle or in later cycles. However, it can occur at other points in the sequencing cycle.

### Methods for Sequencing Complex Nucleic Acids

Overview

According to one embodiment, the present inventions is employed in the context of methods for sequencing target nucleic acids as described herein and, for example, in U.S. Patent Application Publications 2010/0105052 and US2007099208, and U.S. patent application Ser. Nos. 11/679,124 (published as US 2009/0264299); 11/981,761 (US 2009/0155781); 11/981,661 (US 2009/0005252); 11/981,605 (US 2009/0011943); 11/981,793 (US 2009-0118488); 11/451,691 (US 2007/0099208); 11/981,607 (US 2008/0234136); 11/981,767 (US 2009/0137404); 11/982,467 (US 2009/0137414); 11/451,692 (US 2007/0072208); 11/541,225 (US 2010/0081128; 11/927,356 (US 2008/0318796); 11/927,388 (US 2009/0143235); 11/938,096 (US 2008/0213771); 11/938,106 (US 2008/0171331); 10/547,214 (US 2007/0037152); 11/981,730 (US 2009/0005259); 11/981,685 (US 2009/0036316); 11/981,797 (US 2009/0011416); 11/934,695 (US 2009/0075343); 11/934,697 (US 2009/0111705); 11/934,703 (US 2009/0111706); 12/265,593 (US 2009/0203551); 11/938,213 (US 2009/0105961); 11/938,221 (US 2008/0221832); 12/325,922 (US 2009/0318304); 12/252,280 (US 2009/0111115); 12/266,385 (US 2009/0176652); 12/335,168 (US 2009/0311691); 12/335,188 (US 2009/0176234); 12/361,507 (US 2009/0263802), 11/981,804 (US 2011/0004413); and 12/329,365; published international patent application numbers WO2007120208, WO2006073504, and WO2007133831, all of which are hereby incorporated herein by reference in their entirety for all purposes. Exemplary methods for calling variations in a polynucleotide sequence compared to a reference polynucleotide sequence and for polynucleotide sequence assembly (or reassembly), for example, are provided in U.S. patent publication No. 2011-0004413, (application Ser. No. 12/770,089) which is incorporated herein by reference in its entirety for all purposes. See also Drmanac et al., Science 327, 78-81, 2010.

This method includes extracting and fragmenting target nucleic acids from a sample. The fragmented nucleic acids are used to produce library constructs that generally include one or more adaptors. The library constructs are amplified to form amplicons, including in one embodiment concatemeric amplicons referred to herein as “DNA nanoballs” or “DNBs”) that are disposed on a surface. Nucleic acid sequencing is performed on the amplicons, e.g., using a sequencing-by-ligation method called combinatorial probe anchor ligation (“cPAL”). By comparing the resulting sequence information to a reference sequence, sequence variants are identified, including without limitation single nucleotide polymorphisms (SNPs), insertions and deletions (indels), structural variations (SVs), copy number variations (CNVs), etc.

As used herein, the term “complex nucleic acid” refers to a large population of nonidentical nucleic acids or polynucleotides. In certain embodiments, the target nucleic acid is genomic DNA; exome DNA (a subset of whole genomic DNA enriched for transcribed sequences which contains the set of exons in a genome); a transcriptome (i.e., the set of all mRNA transcripts produced in a cell or population of cells, or cDNA produced from such mRNA), a methylome (i.e., the population of methylated sites and the pattern of methylation in a genome); a microbiome; a mixture of genomes of different organisms, a mixture of genomes of different cell types of an organism; and other complex nucleic acid mixtures comprising large numbers of different nucleic acid molecules (examples include, without limitation, a microbiome, a xenograft, a solid tumor biopsy comprising both normal and tumor cells, etc.), including subsets of the aforementioned types of complex nucleic acids. In one embodiment, such a complex nucleic acid has a complete sequence comprising at least one gigabase (Gb) (a diploid human genome comprises approximately 6 Gb of sequence).

Nonlimiting examples of complex nucleic acids include “circulating nucleic acids” (CNA), which are nucleic acids circulating in human blood or other body fluids, including but not limited to lymphatic fluid, liquor, ascites, milk, urine, stool and bronchial lavage, for example, and can be distinguished as either cell-free (CF) or cell-associated nucleic acids (reviewed in Pinzani et al., Methods 50:302-307, 2010), e.g., circulating fetal cells in the bloodstream of a expecting mother (see, e.g., Kavanagh et al., J. Chromatol. B 878:1905-1911, 2010) or circulating tumor cells (CTC) from the bloodstream of a cancer patient (see, e.g., Allard et al., Clin. Cancer Res. 10:6897-6904, 2004). Another example is genomic DNA from a single cell or a small number of cells, such as, for example, from biopsies (e.g., fetal cells biopsied from the trophectoderm of a blastocyst; cancer cells from needle aspiration of a solid tumor; etc.). Another example is pathogens, e.g., bacteria cells, virus, or other pathogens, in a tissue, in blood or other body fluids, etc.

As used herein, the term “target nucleic acid” (or polynucleotide) or “nucleic acid of interest” refers to any nucleic acid (or polynucleotide) suitable for processing and sequencing by the methods described herein. The nucleic acid may be single-stranded or double-stranded and may include DNA, RNA, or other known nucleic acids. The target nucleic acids may be those of any organism, including but not limited to viruses, bacteria, yeast, plants, fish, reptiles, amphibians, birds, and mammals (including, without limitation, mice, rats, dogs, cats, goats, sheep, cattle, horses, pigs, rabbits, monkeys and other non-human primates, and humans). A target nucleic acid may be obtained from an individual or from a multiple individuals (i.e., a population). A sample from which the nucleic acid is obtained may contain a nucleic acids from a mixture of cells or even organisms, such as: a human saliva sample that includes human cells and bacterial cells; a mouse xenograft that includes mouse cells and cells from a transplanted human tumor; etc.

Target nucleic acids may be unamplified or they may be amplified by any suitable nucleic acid amplification method known in the art, including without limitation amplicons generated by the polymerase chain reaction (PCR) (including, for example, two-dimensional PCR, or bridge amplification), strand displacement amplification (SDA), multiple displacement amplification (MDA), rolling circle amplification (RCA), rolling circle replication (RCR), or other well-known amplification methods. Target nucleic acids may be purified according to methods known in the art to remove cellular and subcellular contaminants (lipids, proteins, carbohydrates, nucleic acids other than those to be sequenced, etc.), or they may be unpurified, i.e., include at least some cellular and subcellular contaminants, including without limitation intact cells that are disrupted to release their nucleic acids for processing and sequencing. Target nucleic acids can be obtained from any suitable sample using methods known in the art. Such samples include but are not limited to: tissues, isolated cells or cell cultures, bodily fluids (including, but not limited to, blood, urine, serum, lymph, saliva, anal and vaginal secretions, perspiration and semen); air, agricultural, water and soil samples, etc. In one aspect, the nucleic acid constructs of the invention are formed from genomic DNA.

High coverage in shotgun sequencing is desired because it can overcome errors in base calling and assembly. As used herein, for any given position in an assembled sequence, the term “sequence coverage redundancy,” “sequence coverage” or simply “coverage” means the number of reads representing that position. It can be calculated from the length of the original genome (G), the number of reads (N), and the average read length (L) as N×L/G. Coverage also can be calculated directly by making a tally of the bases for each reference position. For a whole-genome sequence, coverage is expressed as an average for all bases in the assembled sequence. Sequence coverage is the average number of times a base is read (as described above). It is often expressed as “fold coverage,” for example, as in “40× coverage,” meaning that each base in the final assembled sequence is represented on an average of 40 reads.

As used herein, term “call rate” means a comparison of the percent of bases of the complex nucleic acid that are fully called, commonly with reference to a suitable reference sequence such as, for example, a reference genome. Thus, for a whole human genome, the “genome call rate” (or simply “call rate”) is the percent of the bases of the human genome that are fully called with reference to a whole human genome reference. An “exome call rate” is the percent of the bases of the exome that are fully called with reference to an exome reference. An exome sequence may be obtained by sequencing portions of a genome that have been enriched by various known methods that selectively capture genomic regions of interest from a DNA sample prior to sequencing. Alternatively, an exome sequence may be obtained by sequencing a whole human genome, which includes exome sequences. Thus, a whole human genome sequence may have both a “genome call rate” and an “exome call rate.” There is also a “raw read call rate” that reflects the number of bases that get an A/C/G/T designation as opposed to the total number of attempted bases. (Occasionally, the term “coverage” is used in place of “call rate,” but the meaning will be apparent from the context).

DNBs are produced by rolling circle replication in a uniform-temperature, solution-phase reaction with high template concentrations (>20 billion per ml). This approach avoids significant selection bottlenecks and non-clonal amplicons as well as the stochastic inefficiencies of approaches that require precise titration of template concentrations for in situ clonal amplification in emulsion or bridge PCR. These features also allow for automated DNB production of hundreds of genomes per day in standard 96-well plates.

Arrays of the present invention are amenable to relatively inexpensive and efficient imaging techniques. High-occupancy and high-density nanoarrays are self-assembled on photolithography-patterned, solid-phase substrates through electrostatic adsorption of solution-phase DNBs. Such patterned arrays yield a high proportion of informative pixels compared to random-position DNA arrays. Several hundred reaction sites in the compact (˜300 nm diameter in some embodiments) DNB produce bright signals useful for rapid imaging. Such a spot density and resulting image efficiency and reduced reagent consumption enable high sequencing throughput per instrument that can be critical for high scale human genome sequencing for research and clinical applications.

The “unchained” cPAL sequencing biochemistry of the present invention enables inexpensive and accurate base reads. In general, other than the present invention, two different sequencing chemistries are used for contemporary sequencing platforms: sequencing-by-synthesis (SBS) and sequencing-by-ligation (SBL). Both use “chained” reads, wherein the substrate for cycle N+1 is dependent on the product of cycle N; consequently errors may accumulate over multiple cycles and data quality may be affected by errors (especially incomplete extensions) occurring in previous cycles. Thus, these chained sequencing reactions need to be driven to near completion with high concentrations of expensive high purity labeled substrate molecules and enzymes. Thus, the independent, unchained nature of cPAL avoids error accumulation and tolerates low quality bases in otherwise high quality reads, thereby decreasing reagent costs.

Sequencing data generated using methods and compositions of the present invention achieve sufficient quality and accuracy for complete genome association studies, the identification of potentially rare variants associated with disease or therapeutic treatments, and the identification of somatic mutations. The low cost of consumables and efficient imaging enables studies of several hundreds of individuals. The higher accuracy and completeness required for clinical diagnostic applications provides incentive for continued improvement of this and other technologies.

### Preparing Fragments of Genomic Nucleic Acid

Nucleic Acid Isolation

The target genomic DNA is isolated using conventional techniques, for example as disclosed in Sambrook and Russell, Molecular Cloning: A Laboratory Manual, cited supra. In some cases, particularly if small amounts of DNA are employed in a particular step, it is advantageous to provide carrier DNA, e.g. unrelated circular synthetic double-stranded DNA, to be mixed and used with the sample DNA whenever only small amounts of sample DNA are available and there is danger of losses through nonspecific binding, e.g. to container walls and the like.

The term “target nucleic acid” refers to a nucleic acid of interest. In one aspect, target nucleic acids of the invention are genomic nucleic acids, although other target nucleic acids can be used, including mRNA (and corresponding cDNAs, etc.). Target nucleic acids include naturally occurring or genetically altered or synthetically prepared nucleic acids (such as genomic DNA from a mammalian disease model). Target nucleic acids can be obtained from virtually any source and can be prepared using methods known in the art. For example, target nucleic acids can be directly isolated without amplification, isolated by amplification using methods known in the art, including without limitation polymerase chain reaction (PCR), strand displacement amplification (SDA), multiple displacement amplification (MDA), rolling circle amplification (RCA), rolling circle replication (RCR) and other amplification methodologies. Target nucleic acids may also be obtained through cloning, including but not limited to cloning into vehicles such as plasmids, yeast, and bacterial artificial chromosomes.

In some aspects, the target nucleic acids comprise mRNAs or cDNAs. In certain embodiments, the target DNA is created using isolated transcripts from a biological sample. Isolated mRNA may be reverse transcribed into cDNAs using conventional techniques, again as described in Genome Analysis: A Laboratory Manual Series (Vols. I-IV) or Molecular Cloning: A Laboratory Manual.

The target nucleic acids may be single stranded or double-stranded, as specified, or contain portions of both double-stranded or single-stranded sequence. Depending on the application, the nucleic acids may be DNA (including genomic and cDNA), RNA (including mRNA and rRNA) or a hybrid, where the nucleic acid contains any combination of deoxyribo- and ribo-nucleotides, and any combination of bases, including uracil, adenine, thymine, cytosine, guanine, inosine, xathanine hypoxathanine, isocytosine, isoguanine, etc.

By “nucleic acid” or “oligonucleotide” or “polynucleotide” or grammatical equivalents herein means at least two nucleotides covalently linked together. A nucleic acid of the present invention will generally contain phosphodiester bonds, although in some cases, as outlined below (for example in the construction of anchors, primers and probes), nucleic acid analogs are included that may have alternate backbones, comprising, for example, phosphoramide (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphosphoroamidite linkages (see Eckstein, Oligonucleotides and Analogues: A Practical Approach, Oxford University Press), and peptide nucleic acid (also referred to herein as “PNA”) backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with bicyclic structures including locked nucleic acids (also referred to herein as “LNA”), Koshkin et al., J. Am. Chem. Soc. 120:13252 3 (1998); positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins et al., Chem. Soc. Rev. (1995) pp 169 176). Several nucleic acid analogs are described in Rawls, C & E News Jun. 2, 1997 page 35. “Locked nucleic acids” (LNA™) are also included within the definition of nucleic acid analogs. LNAs are a class of nucleic acid analogues in which the ribose ring is “locked” by a methylene bridge connecting the 2′-O atom with the 4′-C atom. All of these references are hereby expressly incorporated by reference in their entirety for all purposes and in particular for all teachings related to nucleic acids. These modifications of the ribose-phosphate backbone may be done to increase the stability and half-life of such molecules in physiological environments. For example, PNA:DNA and LNA-DNA hybrids can exhibit higher stability and thus may be used in some embodiments.

According to some embodiments of the invention, genomic DNA or other complex nucleic acids are obtained from an individual cell or small number of cells with or without purification.

Long fragments are desirable for LFR, for example. Long fragments of genomic nucleic acid can be isolated from a cell by a number of different methods. In one embodiment, cells are lysed and the intact nuclei are pelleted with a gentle centrifugation step. The genomic DNA is then released through proteinase K and RNase digestion for several hours. The material can be treated to lower the concentration of remaining cellular waste, e.g., by dialysis for a period of time (i.e., from 2-16 hours) and/or dilution. Since such methods need not employ many disruptive processes (such as ethanol precipitation, centrifugation, and vortexing), the genomic nucleic acid remains largely intact, yielding a majority of fragments that have lengths in excess of 150 kilobases. In some embodiments, the fragments are from about 5 to about 750 kilobases in lengths. In further embodiments, the fragments are from about 150 to about 600, about 200 to about 500, about 250 to about 400, and about 300 to about 350 kilobases in length. The smallest fragment that can be used for LFR is one containing at least two hets (approximately 2-5 kb), and there is no maximum theoretical size, although fragment length can be limited by shearing resulting from manipulation of the starting nucleic acid preparation. Techniques that produce larger fragments result in a need for fewer aliquots, and those that result in shorter fragments may require more aliquots. Long DNA fragments are isolated and manipulated in a manner that minimizes shearing or absorption of the DNA to a vessel, including, for example, isolating cells in agarose in agarose gel plugs or oil or by using specially coated tubes and plates.

According to embodiments of the invention that employ aliquoting, once the DNA is isolated and before it is aliquoted into individual wells, it is carefully fragmented to avoid loss of material, particularly sequences from the ends of each fragment, since loss of such material can result in gaps in the final genome assembly. In one embodiment, sequence loss is avoided through use of an infrequent nicking enzyme, which creates starting sites for a polymerase, such as phi29 polymerase, at distances of approximately 100 kb from each other. As the polymerase creates a new DNA strand, it displaces the old strand, creating overlapping sequences near the sites of polymerase initiation. As a result, there are very few deletions of sequence.

A controlled use of a 5′ exonuclease (either before or during amplification, e.g., by MDA) can promote multiple replications of the original DNA from a single cell and thus minimize propagation of early errors through copying of copies.

In some embodiments, further duplicating fragmented DNA from the single cell before aliquoting can be achieved by ligating an adaptor with single stranded priming overhang and using an adaptor-specific primer and phi29 polymerase to make two copies from each long fragment. This can generate four cells-worth of DNA from a single cell.

Fragmentation

The target genomic DNA is then fractionated or fragmented to a desired size by conventional techniques including enzymatic digestion, shearing, or sonication, with the latter two finding particular use in the present invention.

Fragment sizes of the target nucleic acid can vary depending on the source target nucleic acid and the library construction methods used, but for standard whole-genome sequencing such fragments typically range from 50 to 600 nucleotides in length. In another embodiment, the fragments are 300 to 600 or 200 to 2000 nucleotides in length. In yet another embodiment, the fragments are 10-100, 50-100, 50-300, 100-200, 200-300, 50-400, 100-400, 200-400, 300-400, 400-500, 400-600, 500-600, 50-1000, 100-1000, 200-1000, 300-1000, 400-1000, 500-1000, 600-1000, 700-1000, 700-900, 700-800, 800-1000, 900-1000, 1500-2000, 1750-2000, and 50-2000 nucleotides in length. Longer fragments are useful for LFR.

In a further embodiment, fragments of a particular size or in a particular range of sizes are isolated. Such methods are well known in the art. For example, gel fractionation can be used to produce a population of fragments of a particular size within a range of basepairs, for example for 500 base pairs+50 base pairs.

In many cases, enzymatic digestion of extracted DNA is not required because shear forces created during lysis and extraction will generate fragments in the desired range. In a further embodiment, shorter fragments (1-5 kb) can be generated by enzymatic fragmentation using restriction endonucleases. In a still further embodiment, about 10 to about 1,000,000 genome-equivalents of DNA ensure that the population of fragments covers the entire genome. Libraries containing nucleic acid templates generated from such a population of overlapping fragments will thus comprise target nucleic acids whose sequences, once identified and assembled, will provide most or all of the sequence of an entire genome.

In some embodiments of the invention, a controlled random enzymatic (“CoRE”) fragmentation method is utilized to prepare fragments. CoRE fragmentation is an enzymatic endpoint assay, and has the advantages of enzymatic fragmentation (such as the ability to use it on low amounts and/or volumes of DNA) without many of its drawbacks (including sensitivity to variation in substrate or enzyme concentration and sensitivity to digestion time).

In one aspect, the present invention provides a method of fragmentation referred to herein as Controlled Random Enzymatic (CoRE) fragmentation, which can be used alone or in combination with other mechanical and enzymatic fragmentation methods known in the art. CoRE fragmentation involves a series of three enzymatic steps. First, a nucleic acid is subjected to an amplification method that is conducted in the present of dNTPs doped with a proportion of deoxyuracil (“dU”) or uracil (“U”) to result in substitution of dUTP or UTP at defined and controllable proportions of the T positions in both strands of the amplification product. Any suitable amplification method can be used in this step of the invention. In certain embodiment, multiple displacement amplification (MDA) in the presence of dNTPs doped with dUTP or UTP in a defined ratio to the dTTP is used to create amplification products with dUTP or UTP substituted into certain points on both strands.

After amplification and insertion of the uracil moieties, the uracils are then excised, usually through a combination of UDG, EndoVIII, and T4PNK, to create single base gaps with functional 5′ phosphate and 3′ hydroxyl ends. The single base gaps will be created at an average spacing defined by the frequency of U in the MDA product. That is, the higher the amount of dUTP, the shorter the resulting fragments. As will be appreciated by those in the art, other techniques that will result in selective replacement of a nucleotide with a modified nucleotide that can similarly result in cleavage can also be used, such as chemically or other enzymatically susceptible nucleotides.

Treatment of the gapped nucleic acid with a polymerase with exonuclease activity results in “translation” or “translocation” of the nicks along the length of the nucleic acid until nicks on opposite strands converge, thereby creating double-strand breaks, resulting a relatively population of double-stranded fragments of a relatively homogenous size. The exonuclease activity of the polymerase (such as Taq polymerase) will excise the short DNA strand that abuts the nick while the polymerase activity will “fill in” the nick and subsequent nucleotides in that strand (essentially, the Taq moves along the strand, excising bases using the exonuclease activity and adding the same bases, with the result being that the nick is translocated along the strand until the enzyme reaches the end).

Since the size distribution of the double-stranded fragments is a result of the ration of dTTP to dUTP or UTP used in the MDA reaction, rather than by the duration or degree of enzymatic treatment, this CoRE fragmentation method produces high degrees of fragmentation reproducibility, resulting in a population of double-stranded nucleic acid fragments that are all of a similar size.

Fragment End Repair and Modification

In certain embodiments, after fragmenting, target nucleic acids are further modified to prepare them for insertion of multiple adaptors according to methods of the invention.

After physical fragmentation, target nucleic acids frequently have a combination of blunt and overhang ends as well as combinations of phosphate and hydroxyl chemistries at the termini. In this embodiment, the target nucleic acids are treated with several enzymes to create blunt ends with particular chemistries. In one embodiment, a polymerase and dNTPs is used to fill in any 5′ single strands of an overhang to create a blunt end. Polymerase with 3′ exonuclease activity (generally but not always the same enzyme as the 5′ active one, such as T4 polymerase) is used to remove 3′ overhangs. Suitable polymerases include, but are not limited to, T4 polymerase, Taq polymerases, E. coli DNA Polymerase 1, Klenow fragment, reverse transcriptases, phi29 related polymerases including wild type phi29 polymerase and derivatives of such polymerases, T7 DNA Polymerase, T5 DNA Polymerase, RNA polymerases. These techniques can be used to generate blunt ends, which are useful in a variety of applications.

In further optional embodiments, the chemistry at the termini is altered to avoid target nucleic acids from ligating to each other. For example, in addition to a polymerase, a protein kinase can also be used in the process of creating blunt ends by utilizing its 3′ phosphatase activity to convert 3′ phosphate groups to hydroxyl groups. Such kinases can include without limitation commercially available kinases such as T4 kinase, as well as kinases that are not commercially available but have the desired activity.

Similarly, a phosphatase can be used to convert terminal phosphate groups to hydroxyl groups. Suitable phosphatases include, but are not limited to, alkaline phosphatase (including calf intestinal phosphatase), antarctic phosphatase, apyrase, pyrophosphatase, inorganic (yeast) thermostable inorganic pyrophosphatase, and the like, which are known in the art.

These modifications prevent the target nucleic acids from ligating to each other in later steps of methods of the invention, thus ensuring that during steps in which adaptors (and/or adaptor arms) are ligated to the termini of target nucleic acids, target nucleic acids will ligate to adaptors but not to other target nucleic acids. Target nucleic acids can be ligated to adaptors in a desired orientation. Modifying the ends avoids the undesired configurations in which the target nucleic acids ligate to each other and/or the adaptors ligate to each other. The orientation of each adaptor-target nucleic acid ligation can also be controlled through control of the chemistry of the termini of both the adaptors and the target nucleic acids. Such modifications can prevent the creation of nucleic acid templates containing different fragments ligated in an unknown conformation, thus reducing and/or removing the errors in sequence identification and assembly that can result from such undesired templates.

The DNA may be denatured after fragmentation to produce single-stranded fragments.

Amplification

In one embodiment, after fragmenting, (and in fact before or after any step outlined herein) an amplification step can be applied to the population of fragmented nucleic acids to ensure that a large enough concentration of all the fragments is available for subsequent steps. According to one embodiment of the invention, methods are provided for sequencing small quantities of complex nucleic acids, including those of higher organisms, in which such complex nucleic acids are amplified in order to produce sufficient nucleic acids for sequencing by the methods described herein. Sequencing methods described herein provide highly accurate sequences at a high call rate even with a fraction of a genome equivalent as the starting material with sufficient amplification. Note that a cell includes approximately 6.6 picograms (pg) of genomic DNA. Whole genomes or other complex nucleic acids from single cells or a small number of cells of an organism, including higher organisms such as humans, can be performed by the methods of the present invention. Sequencing of complex nucleic acids of a higher organism can be accomplished using 1 pg, 5 pg, 10 pg, 30 pg, 50 pg, 100 pg, or 1 ng of a complex nucleic acid as the starting material, which is amplified by any nucleic acid amplification method known in the art, to produce, for example, 200 ng, 400 ng, 600 ng, 800 ng, 1 μg, 2 μg, 3 μg, 4 μg, 5 μg, 10 μg or greater quantities of the complex nucleic acid. We also disclose nucleic acid amplification protocols that minimize GC bias. However, the need for amplification and subsequent GC bias can be reduced further simply by isolating one cell or a small number of cells, culturing them for a sufficient time under suitable culture conditions known in the art, and using progeny of the starting cell or cells for sequencing.

Such amplification methods include without limitation: multiple displacement amplification (MDA), polymerase chain reaction (PCR), ligation chain reaction (sometimes referred to as oligonucleotide ligase amplification OLA), cycling probe technology (CPT), strand displacement assay (SDA), transcription mediated amplification (TMA), nucleic acid sequence based amplification (NASBA), rolling circle amplification (RCA) (for circularized fragments), and invasive cleavage technology.

Amplification can be performed after fragmenting or before or after any step outlined herein.

MDA Amplification Protocol with Reduced GC Bias

In one aspect, the present invention provides methods of sample of preparation in which ˜10 Mb of DNA per aliquot is faithfully amplified, e.g., approximately 30,000-fold depending on the amount of starting DNA, prior to library construction and sequencing.

According to one embodiment of LFR methods of the present invention, LFR begins with treatment of genomic nucleic acids, usually genomic DNA, with a 5′ exonuclease to create 3′ single-stranded overhangs. Such single stranded overhangs serve as MDA initiation sites. Use of the exonuclease also eliminates the need for a heat or alkaline denaturation step prior to amplification without introducing bias into the population of fragments. In another embodiment, alkaline denaturation is combined with the 5′ exonuclease treatment, which results in a reduction in bias that is greater than what is seen with either treatment alone. DNA treated with 5′ exonuclease and optionally with alkaline denaturation is then diluted to sub-genome concentrations and dispersed across a number of aliquots, as discussed above. After separation into aliquots, e.g., across multiple wells, the fragments in each aliquot are amplified.

In one embodiment, a phi29-based multiple displacement amplification (MDA) is used. Numerous studies have examined the range of unwanted amplification biases, background product formation, and chimeric artifacts introduced via phi29 based MDA, but many of these short comings have occurred under extreme conditions of amplification (greater than 1 million fold). Commonly, LFR employs a substantially lower level of amplification and starts with long DNA fragments (e.g., ˜100 kb), resulting in efficient MDA and a more acceptable level of amplification biases and other amplification-related problems.

We have developed an improved MDA protocol to overcome problems associated with MDA that uses various additives (e.g., DNA modifying enzymes, sugars, and/or chemicals like DMSO), and/or different components of the reaction conditions for MDA are reduced, increased or substituted to further improve the protocol. To minimize chimeras, reagents can also be included to reduce the availability of the displaced single stranded DNA from acting as an incorrect template for the extending DNA strand, which is a common mechanism for chimera formation. A major source of coverage bias introduced by MDA is caused by differences in amplification between GC-rich verses AT-rich regions. This can be corrected by using different reagents in the MDA reaction and/or by adjusting the primer concentration to create an environment for even priming across all % GC regions of the genome. In some embodiments, random hexamers are used in priming MDA. In other embodiments, other primer designs are utilized to reduce bias. In further embodiments, use of 5′ exonuclease before or during MDA can help initiate low-bias successful priming, particularly with longer (i.e., 200 kb to 1 Mb) fragments that are useful for sequencing regions characterized by long segmental duplication (i.e., in some cancer cells) and complex repeats.

In some embodiments, improved, more efficient fragmentation and ligation steps are used that reduce the number of rounds of MDA amplification required for preparing samples by as much as 10,000 fold, which further reduces bias and chimera formation resulting from MDA.

In some embodiments, the MDA reaction is designed to introduce uracils into the amplification products in preparation for CoRE fragmentation. In some embodiments, a standard MDA reaction utilizing random hexamers is used to amplify the fragments in each well; alternatively, random 8-mer primers can be used to reduce amplification bias (e.g., GC-bias) in the population of fragments. In further embodiments, several different enzymes can also be added to the MDA reaction to reduce the bias of the amplification. For example, low concentrations of non-processive 5′ exonucleases and/or single-stranded binding proteins can be used to create binding sites for the 8-mers. Chemical agents such as betaine, DMSO, and trehalose can also be used to reduce bias.

After amplification of the fragments in each aliquot, the amplification products may optionally be subjected to another round of fragmentation. In some embodiments the CoRE method is used to further fragment the fragments in each aliquot following amplification. In such embodiments, MDA amplification of fragments in each aliquot is designed to incorporate uracils into the MDA products. Each aliquot containing MDA products is treated with a mix of Uracil DNA glycosylase (UDG), DNA glycosylase-lyase Endonuclease VIII, and T4 polynucleotide kinase to excise the uracil bases and create single base gaps with functional 5′ phosphate and 3′ hydroxyl groups. Nick translation through use of a polymerase such as Taq polymerase results in double-stranded blunt-end breaks, resulting in ligatable fragments of a size range dependent on the concentration of dUTP added in the MDA reaction. In some embodiments, the CoRE method used involves removing uracils by polymerization and strand displacement by phi29. The fragmenting of the MDA products can also be achieved via sonication or enzymatic treatment. Enzymatic treatment that could be used in this embodiment includes without limitation DNase I, T7 endonuclease I, micrococcal nuclease, and the like.

Following fragmentation of the MDA products, the ends of the resultant fragments may be repaired. Many fragmentation techniques can result in termini with overhanging ends and termini with functional groups that are not useful in later ligation reactions, such as 3′ and 5′ hydroxyl groups and/or 3′ and 5′ phosphate groups. It may be useful to have fragments that are repaired to have blunt ends. It may also be desirable to modify the termini to add or remove phosphate and hydroxyl groups to prevent “polymerization” of the target sequences. For example, a phosphatase can be used to eliminate phosphate groups, such that all ends contain hydroxyl groups. Each end can then be selectively altered to allow ligation between the desired components. One end of the fragments can then be “activated” by treatment with alkaline phosphatase. The fragments then can be tagged with an adaptor to identify fragments that come from the same aliquot in the LFR method.

Tagging Fragments in Each Aliquot

According to one embodiment, after amplification, the DNA in each aliquot is tagged so as to identify the aliquot in which each fragment originated. In further embodiments the amplified DNA in each aliquot is further fragmented before being tagged with an adaptor such that fragments from the same aliquot will all comprise the same tag; see for example US 2007/0072208, hereby incorporated by reference.

According to one embodiment, the adaptor is designed in two segments—one segment is common to all wells and blunt end ligates directly to the fragments using methods described further herein. The “common” adaptor is added as two adaptor arms—one arm is blunt end ligated to the 5′ end of the fragment and the other arm is blunt end ligated to the 3′ end of the fragment. The second segment of the tagging adaptor is a “barcode” segment that is unique to each well. This barcode is generally a unique sequence of nucleotides, and each fragment in a particular well is given the same barcode. Thus, when the tagged fragments from all the wells are re-combined for sequencing applications, fragments from the same well can be identified through identification of the barcode adaptor. The barcode is ligated to the 5′ end of the common adaptor arm. The common adaptor and the barcode adaptor can be ligated to the fragment sequentially or simultaneously. As will be described in further detail herein, the ends of the common adaptor and the barcode adaptor can be modified such that each adaptor segment will ligate in the correct orientation and to the proper molecule. Such modifications prevent “polymerization” of the adaptor segments or the fragments by ensuring that the fragments are unable to ligate to each other and that the adaptor segments are only able to ligate in the illustrated orientation.

In further embodiments, a three segment design is utilized for the adaptors used to tag fragments in each well. This embodiment is similar to the barcode adaptor design described above, except that the barcode adaptor segment is split into two segments. This design allows for a wider range of possible barcodes by allowing combinatorial barcode adaptor segments to be generated by ligating different barcode segments together to form the full barcode segment. This combinatorial design provides a larger repertoire of possible barcode adaptors while reducing the number of full size barcode adaptors that need to be generated. In further embodiments, unique identification of each aliquot is achieved with 8-12 base pair error correcting barcodes. In some embodiments, the same number of adaptors as wells (384 and 1536 in the above-described non-limiting examples) is used. In further embodiments, the costs associated with generating adaptors is are reduced through a novel combinatorial tagging approach based on two sets of 40 half-barcode adapters.

In one embodiment, library construction involves using two different adaptors. A and B adapters are easily be modified to each contain a different half-barcode sequence to yield thousands of combinations. In a further embodiment, the barcode sequences are incorporated on the same adapter. This can be achieved by breaking the B adaptor into two parts, each with a half barcode sequence separated by a common overlapping sequence used for ligation. The two tag components have 4-6 bases each. An 8-base (2×4 bases) tag set is capable of uniquely tagging 65,000 aliquots. One extra base (2×5 bases) will allow error detection and 12 base tags (2×6 bases, 12 million unique barcode sequences) can be designed to allow substantial error detection and correction in 10,000 or more aliquots using Reed-Solomon design (U.S. patent application Ser. No. 12/697,995, published as US 2010/0199155, which is incorporated herein by reference). Both 2×5 base and 2×6 base tags may include use of degenerate bases (i.e., “wild-cards”) to achieve optimal decoding efficiency.

After the fragments in each well are tagged, all of the fragments are combined or pooled to form a single population. These fragments can then be used to generate nucleic acid templates or library constructs for sequencing. The nucleic acid templates generated from these tagged fragments will be identifiable as belonging to a particular well by the barcode tag adaptors attached to each fragment.

### Library Constructs

Overview

The present invention provides library constructs comprising target nucleic acids and multiple interspersed adaptors. These constructs are created by inserting adaptors molecules at a multiplicity of sites throughout each target nucleic acid. The interspersed adaptors permit acquisition of sequence information from multiple sites in the target nucleic acid consecutively or simultaneously.

The nucleic acid templates (also referred to herein as “nucleic acid constructs” and “library constructs”) of the invention comprise target nucleic acids and adaptors. As used herein, the term “adaptor” refers to an oligonucleotide of known sequence. Adaptors of use in the present invention may include a number of elements. The types and numbers of elements (also referred to herein as “features”) included in an adaptor will depend on the intended use of the adaptor. Adaptors of use in the present invention will generally include without limitation sites for restriction endonuclease recognition and/or cutting, particularly Type IIs recognition sites that allow for endonuclease binding at a recognition site within the adaptor and cutting outside the adaptor as described below, sites for primer binding (for amplifying the nucleic acid constructs) or anchor binding (for sequencing the target nucleic acids in the nucleic acid constructs), nickase sites, and the like. In some embodiments, adaptors will comprise a single recognition site for a restriction endonuclease, whereas in other embodiments, adaptors will comprise two or more recognition sites for one or more restriction endonucleases. As outlined herein, the recognition sites are frequently (but not exclusively) found at the termini of the adaptors, to allow cleavage of the double-stranded constructs at the farthest possible position from the end of the adaptor.

In some embodiments, adaptors of the invention have a length of about 10 to about 250 nucleotides, depending on the number and size of the features included in the adaptors. In certain embodiments, adaptors of the invention have a length of about 50 nucleotides. In further embodiments, adaptors of use in the present invention have a length of about 20 to about 225, about 30 to about 200, about 40 to about 175, about 50 to about 150, about 60 to about 125, about 70 to about 100, and about 80 to about 90 nucleotides.

In further embodiments, adaptors may optionally include elements such that they can be ligated to a target nucleic acid as two “arms”. One or both of these arms may comprise an intact recognition site for a restriction endonuclease, or both arms may comprise part of a recognition site for a restriction endonuclease. In the latter case, circularization of a construct comprising a target nucleic acid bounded at each termini by an adaptor arm will reconstitute the entire recognition site.

In still further embodiments, adaptors of use in the invention will comprise different anchor binding sites at their 5′ and the 3′ ends of the adaptor. As described further herein, such anchor binding sites can be used in sequencing applications, including the combinatorial probe-anchor ligation (cPAL) method of sequencing, described herein and in U.S. Application Ser. Nos. 60/992,485; 61/026,337; 61/035,914; 61/061,134; 61/116,193; 61/102,586; 12/265,593; and 12/266,385 11/938,106; 11/938,096; 11/982,467; 11/981,804; 11/981,797; 11/981,793; 11/981,767; 11/981,761; 11/981,730; 11/981,685; 11/981,661; 11/981,607; 11/981,605; 11/927,388; 11/927,356; 11/679,124; 11/541,225;10/547,214; and 11/451,691, all of which are hereby incorporated by reference in their entirety, and particularly for disclosure relating to sequencing by ligation.

In one aspect, adaptors of the invention are interspersed adaptors. By “interspersed adaptors” is meant herein oligonucleotides that are inserted at spaced locations within the interior region of a target nucleic acid. In one aspect, “interior” in reference to a target nucleic acid means a site internal to a target nucleic acid prior to processing, such as circularization and cleavage, that may introduce sequence inversions, or like transformations, which disrupt the ordering of nucleotides within a target nucleic acid.

The nucleic acid template constructs of the invention contain multiple interspersed adaptors inserted into a target nucleic acid, and in a particular orientation. As discussed further herein, the target nucleic acids are produced from nucleic acids isolated from one or more cells, including one to several million cells. These nucleic acids are then fragmented using mechanical or enzymatic methods.

The target nucleic acid that becomes part of a nucleic acid template construct of the invention may have interspersed adaptors inserted at intervals within a contiguous region of the target nucleic acids at predetermined positions. The intervals may or may not be equal. In some aspects, the accuracy of the spacing between interspersed adaptors may be known only to an accuracy of one to a few nucleotides. In other aspects, the spacing of the adaptors is known, and the orientation of each adaptor relative to other adaptors in the library constructs is known. That is, in many embodiments, the adaptors are inserted at known distances, such that the target sequence on one termini is contiguous in the naturally occurring genomic sequence with the target sequence on the other termini. For example, in the case of a Type IIs restriction endonuclease that cuts 16 bases from the recognition site, located 3 bases into the adaptor, the endonuclease cuts 13 bases from the end of the adaptor. Upon the insertion of a second adaptor, the target sequence “upstream” of the adaptor and the target sequence “downstream” of the adaptor are actually contiguous sequences in the original target sequence. These “mate paired” sequences extend the number of contiguous reads possible from a construct, and are of particular use in reading through repetitive elements in genomes.

Although the embodiments of the invention described herein are generally described in terms of circular nucleic acid template constructs, it will be appreciated that nucleic acid template constructs may also be linear. Furthermore, nucleic acid template constructs of the invention may be single- or double-stranded, with the latter being preferred in some embodiments

The present invention provides nucleic acid templates comprising a target nucleic acid containing one or more interspersed adaptors. In a further embodiment, nucleic acid templates formed from a plurality of genomic fragments can be used to create a library of nucleic acid templates. Such libraries of nucleic acid templates will in some embodiments encompass target nucleic acids that together encompass all or part of an entire genome. That is, by using a sufficient number of starting genomes (e.g. cells), combined with random fragmentation, the resulting target nucleic acids of a particular size that are used to create the circular templates of the invention sufficiently “cover” the genome, although as will be appreciated, on occasion, bias may be introduced inadvertently to prevent the entire genome from being represented.

The nucleic acid template constructs of the invention comprise multiple interspersed adaptors, and in some aspects, these interspersed adaptors comprise one or more recognition sites for restriction endonucleases. In further aspect, the adaptors comprise recognition sites for Type IIs endonucleases. Type-IIs endonucleases are generally commercially available and are well known in the art. Like their Type-II counterparts, Type-IIs endonucleases recognize specific sequences of nucleotide base pairs within a double-stranded polynucleotide sequence. Upon recognizing that sequence, the endonuclease will cleave the polynucleotide sequence, generally leaving an overhang of one strand of the sequence, or “sticky end.” Type-IIs endonucleases also generally cleave outside of their recognition sites; the distance may be anywhere from about 2 to 30 nucleotides away from the recognition site depending on the particular endonuclease. Some Type-IIs endonucleases are “exact cutters” that cut a known number of bases away from their recognition sites. In some embodiments, Type IIs endonucleases are used that are not “exact cutters” but rather cut within a particular range (e.g. 6 to 8 nucleotides). Generally, Type IIs restriction endonucleases of use in the present invention have cleavage sites that are separated from their recognition sites by at least six nucleotides (i.e. the number of nucleotides between the end of the recognition site and the closest cleavage point). Exemplary Type IIs restriction endonucleases include, but are not limited to, Eco57M I, Mme I, Acu I, Bpm I, BceA I, Bbv I, BciV I, BpuE I, BseM II, BseR I, Bsg I, BsmF I, BtgZ I, Eci I, EcoP15 I, Eco57M I, Fok I, Hga I, Hph I, Mbo II, Mnl I, SfaN I, TspDT I, TspDW I, Taq II, and the like. In some exemplary embodiments, the Type IIs restriction endonucleases used in the present invention are AcuI, which has a cut length of about 16 bases with a 2-base 3′ overhang and EcoP15, which has a cut length of about 25 bases with a 2-base 5′ overhang. As will be discussed further below, the inclusion of a Type IIs site in the adaptors of the nucleic acid template constructs of the invention provides a tool for inserting multiple adaptors in a target nucleic acid at a defined location.

As will be appreciated, adaptors may also comprise other elements, including recognition sites for other (non-Type IIs) restriction endonucleases, primer binding sites for amplification as well as binding sites for anchors used in sequencing reactions, described further herein.

In one aspect, adaptors of use in the invention can comprise multiple functional features, including recognition sites for Type IIs restriction endonucleases, sites for nicking endonucleases, sequences that can influence secondary characteristics, such as bases to disrupt hairpins, etc. Adaptors of use in the invention may in addition contain palindromic sequences, which can serve to promote intramolecular binding once nucleic acid templates comprising such adaptors are used to generate concatemers.

Preparing Nucleic Acid Templates of the Invention

Methods for preparing library constructs is described in detail, for example, in U.S. Patent Application Publications 2010/0105052 and US2007099208, and U.S. patent application Ser. Nos. 11/679,124 (published as US 2009/0264299); 11/981,761 (US 2009/0155781); 11/981,661 (US 2009/0005252); 11/981,605 (US 2009/0011943); 11/981,793 (US 2009-0118488); 11/451,691 (US 2007/0099208); 11/981,607 (US 2008/0234136); 11/981,767 (US 2009/0137404); 11/982,467 (US 2009/0137414); 11/451,692 (US 2007/0072208); 11/541,225 (US 2010/0081128; 11/927,356 (US 2008/0318796); 11/927,388 (US 2009/0143235); 11/938,096 (US 2008/0213771); 11/938,106 (US 2008/0171331); 10/547,214 (US 2007/0037152); 11/981,730 (US 2009/0005259); 11/981,685 (US 2009/0036316); 11/981,797 (US 2009/0011416); 11/934,695 (US 2009/0075343); 11/934,697 (US 2009/0111705); 11/934,703 (US 2009/0111706); 12/265,593 (US 2009/0203551); 11/938,213 (US 2009/0105961); 11/938,221 (US 2008/0221832); 12/325,922 (US 2009/0318304); 12/252,280 (US 2009/0111115); 12/266,385 (US 2009/0176652); 12/335,168 (US 2009/0311691); 12/335,188 (US 2009/0176234); 12/361,507 (US 2009/0263802), 11/981,804 (US 2011/0004413); and 12/329,365; published international patent application numbers WO2007120208, WO2006073504, and WO2007133831, all of which are incorporated herein by reference in their entirety for all purposes. See also Drmanac et al., Science 327, 78-81, 2010. The following provides a summary of examples of such methods.

Overview of Generation of Circular Templates

The present invention is directed to compositions and methods for nucleic acid identification and detection, which finds use in a wide variety of applications as described herein, including a variety of sequencing and genotyping applications. The methods described herein allow the construction of circular nucleic acid templates that are used in amplification reactions that utilize such circular templates to create concatamers of the monomeric circular templates, forming “DNA nanoballs”, described below, which find use in a variety of sequencing and genotyping applications. The circular or linear constructs of the invention comprise target nucleic acid sequences, generally fragments of genomic DNA (although as described herein, other templates such as cDNA can be used), with interspersed exogeneous nucleic acid adaptors. The present invention provides methods for producing nucleic acid template constructs in which each subsequent adaptor is added at a defined position and also optionally in a defined orientation in relation to one or more previously inserted adaptors. These nucleic acid template constructs are generally circular nucleic acids (although in certain embodiments the constructs can be linear) that include target nucleic acids with multiple interspersed adaptors. These adaptors, as described below, are exogenous sequences used in the sequencing and genotyping applications, and usually contain a restriction endonuclease site, particularly for enzymes such as Type IIs enzymes that cut outside of their recognition site. For ease of analysis, the reactions of the invention preferably utilize embodiments where the adaptors are inserted in particular orientations, rather than randomly. Thus the invention provides methods for making nucleic acid constructs that contain multiple adaptors in particular orientations and with defined spacing between them.

In nucleic acid template constructs comprising multiple adaptors, at least one of the adaptors will be inserted into contiguous nucleotides of the target nucleic acid, so that reads from each end of these inserted (also referred to herein as “interspersed”) adaptors results in a read of contiguous bases. For example, 10-base reads from each end of an interspersed adaptor provides a read of 20 contiguous bases of the target nucleic acid.

Control over the spacing and orientation of insertion of each subsequent adaptor provides a number of advantages over random insertion of interspersed adaptors. In particular, the methods described herein improve the efficiency of the adaptor insertion process, thus reducing the need to introduce amplification steps as each subsequent adaptor is inserted. In addition, controlling the spacing and orientation of each added adaptor ensures that the restriction endonuclease recognition sites that are generally included in each adaptor are positioned to allow subsequent cleavage and ligation steps to occur at the proper point in the nucleic acid construct, thus further increasing efficiency of the process by reducing or eliminating the formation of nucleic acid templates that have adaptors in the improper location or orientation. In addition, control over location and orientation of each subsequently added adaptor can be beneficial to certain uses of the resultant nucleic acid construct, because the adaptors serve a variety of functions in sequencing applications, including serving as a reference point of known sequence to aid in identifying the relative spatial location of bases identified at certain positions within the target nucleic acid. Such uses of adaptors in sequencing applications are described further herein.

Genomic nucleic acid, generally double-stranded DNA, is obtained from one or more cells, generally from about 5, 100, or 1000 or more cells. The genomic nucleic acid is fractionated into appropriate sizes using standard techniques such as physical or enzymatic fractionation combined with size fractionation.

In addition, as needed, amplification can also optionally be conducted using a wide variety of known techniques to increase the number of genomic fragments for further manipulation, although in many embodiments, an amplification step is not needed at this step.

Adding a First Adaptor

As a first step in the creation of nucleic acid templates of the invention, a first adaptor is ligated to a target nucleic acid. The entire first adaptor may be added to one terminus, or two portions of the first adaptor, referred to herein as “adaptor arms”, can be ligated to each terminus of the target nucleic acid. The first adaptor arms are designed such that upon ligation they reconstitute the entire first adaptor. As described further above, the first adaptor will generally comprise one or more recognition sites for a Type IIs restriction endonuclease. In some embodiments, a Type IIs restriction endonuclease recognition site will be split between the two adaptor arms, such that the site is only available for binding to a restriction endonuclease upon ligation of the two adaptor arms.

According to one method for assembling adaptor/target nucleic acid templates (also referred to herein as “target library constructs”, “library constructs” and all grammatical equivalents), DNA, such as genomic DNA, is isolated and fragmented into target nucleic acids using standard techniques as described above. The fragmented target nucleic acids are then repaired so that the 5′ and 3′ ends of each strand are flush or blunt ended. Following this reaction, each fragment is “A-tailed” with a single A added to the 3′ end of each strand of the fragmented target nucleic acids using a non-proofreading polymerase. The A-tailing is generally accomplished by using a polymerase (such as Taq polymerase) and providing only adenosine nucleotides, such that the polymerase is forced to add one or more A's to the end of the target nucleic acid in a template-sequence-independent manner.

In an exemplary method, a first and second arm of a first adaptor is then ligated to each target nucleic acid, producing a target nucleic acid with adaptor arms ligated to each end. In one embodiment, the adaptor arms are “T tailed” to be complementary to the A tails of the target nucleic acid, facilitating ligation of the adaptor arms to the target nucleic acid by providing a way for the adaptor arms to first anneal to the target nucleic acids and then applying a ligase to join the adaptor arms to the target nucleic acid.

In a further embodiment, the invention provides adaptor ligation to each fragment in a manner that minimizes the creation of intra- or intermolecular ligation artifacts. This is desirable because random fragments of target nucleic acids forming ligation artifacts with one another create false proximal genomic relationships between target nucleic acid fragments, complicating the sequence alignment process. Using both A tailing and T tailing to attach the adaptor to the DNA fragments prevents random intra- or inter-molecular associations of adaptors and fragments, which reduces artifacts that would be created from self-ligation, adaptor-adaptor or fragment-fragment ligation.

As an alternative to A/T tailing (or G/C tailing), various other methods can be implemented to prevent formation of ligation artifacts of the target nucleic acids and the adaptors, as well as orient the adaptor arms with respect to the target nucleic acids, including using complementary NN overhangs in the target nucleic acids and the adaptor arms, or employing blunt end ligation with an appropriate target nucleic acid to adaptor ratio to optimize single fragment nucleic acid/adaptor arm ligation ratios.

After creating a linear construct comprising a target nucleic acid and with an adaptor arm on each terminus, the linear target nucleic acid is circularized, a process that will be discussed in further detail herein, resulting in a circular construct comprising target nucleic acid and an adaptor. Note that the circularization process results in bringing the first and second arms of the first adaptor together to form a contiguous first adaptor in the circular construct. In some embodiments, the circular construct is amplified, such as by circle dependent amplification, using, e.g., random hexamers and phi29 or helicase. Alternatively, target nucleic acid/adaptor structure may remain linear, and amplification may be accomplished by PCR primed from sites in the adaptor arms. The amplification preferably is a controlled amplification process and uses a high fidelity, proof-reading polymerase, resulting in a sequence-accurate library of amplified target nucleic acid/adaptor constructs where there is sufficient representation of the genome or one or more portions of the genome being queried.

Adding Multiple Adaptors

According to one method for assembling adaptor/target nucleic acid templates (also referred to herein as “target library constructs”, “library constructs” and all grammatical equivalents). DNA, such as genomic DNA, is isolated and fragmented into target nucleic acids using standard techniques. The fragmented target nucleic acids are then in some embodiments repaired so that the 5′ and 3′ ends of each strand are flush or blunt ended.

In one method, a first and second arm of a first adaptor is ligated to each target nucleic acid, producing a target nucleic acid with adaptor arms ligated to each end.

After creating a linear construct comprising a target nucleic acid and with an adaptor arm on each terminus, the linear target nucleic acid is circularized, a process that will be discussed in further detail herein, resulting in a circular construct comprising target nucleic acid and an adaptor. Note that the circularization process results in bringing the first and second arms of the first adaptor together to form a contiguous first adaptor in the circular construct. In some embodiments, the circular construct is amplified, such as by circle dependent amplification, using, e.g., random hexamers and phi29 or helicase. Alternatively, target nucleic acid/adaptor structure may remain linear, and amplification may be accomplished by PCR primed from sites in the adaptor arms. The amplification preferably is a controlled amplification process and uses a high fidelity, proof-reading polymerase, resulting in a sequence-accurate library of amplified target nucleic acid/adaptor constructs where there is sufficient representation of the genome or one or more portions of the genome being queried.

Similar to the process for adding the first adaptor, a second set of adaptor arms and can be added to each end of the linear molecule and then ligated to form the full adaptor and circular molecule. Again, a third adaptor can be added to the other side of adaptor by utilizing a Type IIs endonuclease that cleaves on the other side of adaptor and then ligating a third set of adaptor arms to each terminus of the linearized molecule. Finally, a fourth adaptor can be added by again cleaving the circular construct and adding a fourth set of adaptor arms to the linearized construct. In one method, Type IIs endonucleases with recognition sites in adaptors are applied to cleave the circular construct. The recognition sites in adaptors may be identical or different. Similarly, the recognition sites in all of the adaptors may be identical or different.

A circular construct comprising a first adaptor may contain two Type IIs restriction endonuclease recognition sites in that adaptor, positioned such that the target nucleic acid outside the recognition sequence (and outside of the adaptor) is cut. In one process, EcoP15, a Type IIs restriction endonuclease, is used to cut the circular construct. A portion of each library construct mapping to a portion of the target nucleic acid will be cut away from the construct. Restriction of the library constructs with EcoP15 in the process results in a library of linear constructs containing the first adaptor, with the first adaptor “interior” to the ends of the linear construct. The resulting linear library construct will have a size defined by the distance between the endonuclease recognition sites and the endonuclease restriction site plus the size of the adaptor. In this process, the linear construct, like the fragmented target nucleic acid, is treated by conventional methods to become blunt or flush ended, A tails comprising a single A are added to the 3′ ends of the linear library construct using a non-proofreading polymerase and first and second arms of a second adaptor are ligated to ends of the linearized library construct by A-T tailing and ligation. The resulting library construct comprises a structure with the first adaptor interior to the ends of the linear construct, with target nucleic acid flanked on one end by the first adaptor, and on the other end by either the first or second arm of the second adaptor.

In one process, the double-stranded linear library constructs are treated so as to become single-stranded, and the single-stranded library constructs are then ligated to form single-stranded circles of target nucleic acid interspersed with two adaptors. The ligation/circularization process is performed under conditions that optimize intramolecular ligation. At certain concentrations and reaction conditions, the local intramolecular ligation of the ends of each nucleic acid construct is favored over ligation between molecules.

In some embodiments, 2, 3, 4, 5, 6, 7, 8, 9 or 10 adaptors are included in nucleic acid templates of the invention, with each adapter being independently selected such that they can be all the same, all different, or have sets of the same adapters (e.g. two adapters having the same sequence, two having the same but different sequences, with all combinations possible as described herein). As is described herein, any number of restriction endonucleases can be used, and they can be the same or different depending on the format of the system. Each directionally inserted adaptor substantially extends the read length of SBS or SBL in addition to cPAL.

### Making DNBs

In one aspect, nucleic acid templates of the invention are used to generate nucleic acid nanoballs, which are also referred to herein as “DNA nanoballs,” “DNBs”, and “amplicons”. These nucleic acid nanoballs are generally concatemers comprising multiple copies of a monomer unit consisting of the sequence of a circular library construct. In general, this amplification process is performed in solution in a single reaction chamber, allowing for higher density and lower reagent usage. In addition, since DNB production produces clonal amplicons, this amplification method is generally not subject to stochastic variation from limiting dilution that is inherent in other approaches. Methods of producing DNBs according to the present invention can generate over 10 billion DNBs in one milliliter of reaction volume, which is sufficient for sequencing an entire human genome.

In one aspect, rolling circle replication (RCR) is used to create concatemers of the invention. The RCR process has been shown to generate multiple continuous copies of the M13 genome. (Blanco, et al., (1989) J Biol Chem 264:8935-8940). In such a method, a nucleic acid is replicated by linear concatemerization. Guidance for selecting conditions and reagents for RCR reactions is available in many references available to those of ordinary skill, including U.S. Pat. Nos. 5,426,180; 5,854,033; 6,143,495; and 5,871,921, each of which is hereby incorporated by reference in its entirety for all purposes and in particular for all teachings related to generating concatemers using RCR or other methods.

Generally, RCR reaction components include single stranded DNA circles, one or more primers that anneal to DNA circles, a DNA polymerase having strand displacement activity to extend the 3′ ends of primers annealed to DNA circles, nucleoside triphosphates, and a conventional polymerase reaction buffer. Such components are combined under conditions that permit primers to anneal to DNA circle. Extension of these primers by the DNA polymerase forms concatemers of DNA circle complements. In some embodiments, nucleic acid templates of the invention are double-stranded circles that are denatured to form single stranded circles that can be used in RCR reactions.

In some embodiments, amplification of circular nucleic acids may be implemented by successive ligation of short oligonucleotides, e.g., 6-mers, from a mixture containing all possible sequences, or if circles are synthetic, a limited mixture of these short oligonucleotides having selected sequences for circle replication, a process known as “circle dependent amplification” (CDA). “Circle dependent amplification” or “CDA” refers to multiple displacement amplification of a double-stranded circular template using primers annealing to both strands of the circular template to generate products representing both strands of the template, resulting in a cascade of multiple-hybridization, primer-extension and strand-displacement events. This leads to an exponential increase in the number of primer binding sites, with a consequent exponential increase in the amount of product generated over time. The primers used may be of a random sequence (e.g., random hexamers) or may have a specific sequence to select for amplification of a desired product. CDA results in a set of concatemeric double-stranded fragments being formed.

Concatemers may also be generated by ligation of target DNA in the presence of a bridging template DNA complementary to both beginning and end of the target molecule. A population of different target DNA may be converted in concatemers by a mixture of corresponding bridging templates.

In some embodiments, a subset of a population of nucleic acid templates may be isolated based on a particular feature, such as a desired number or type of adaptor. This population can be isolated or otherwise processed (e.g., size selected) using conventional techniques, e.g., a conventional spin column, or the like, to form a population from which a population of concatemers can be created using techniques such as RCR.

Methods for forming DNBs of the invention are described in Published Patent Application Nos. WO2007120208, WO2006073504, WO2007133831, and US2007099208, and U.S. Patent Application Ser. Nos. 60/992,485; 61/026,337; 61/035,914; 61/061,134; 61/116,193; 61/102,586; 12/265,593; 12/266,385; 11/938,096; 11/981,804; 11/981,797; 11/981,793; 11/981,767; 11/981,761; 11/981,730, filed Oct. 31, 2007; 11/981,685; 11/981,661; 11/981,607; 11/981,605; 11/927,388; 11/927,356; 11/679,124; 11/541,225; 10/547,214; 11/451,692; and 11/451,691, all of which are incorporated herein by reference in their entirety for all purposes and in particular for all teachings related to forming DNBs.

### Producing Arrays of DNBs

In one aspect, DNBs of the invention are disposed on a surface to form a random array of single molecules. DNBs can be fixed to surface by a variety of techniques, including covalent attachment and non-covalent attachment. In one embodiment, a surface may include capture probes that form complexes, e.g., double-stranded duplexes, with component of a polynucleotide molecule, such as an adaptor oligonucleotide. In other embodiments, capture probes may comprise oligonucleotide clamps, or like structures, that form triplexes with adaptors, as described in Gryaznov et al, U.S. Pat. No. 5,473,060, which is hereby incorporated in its entirety.

Methods for forming arrays of DNBs of the invention are described in Published Patent Application Nos. WO2007120208, WO2006073504, WO2007133831, and US2007099208, and U.S. Patent Application Ser. Nos. 60/992,485; 61/026,337; 61/035,914; 61/061,134; 61/116,193; 61/102,586; 12/265,593; 12/266,385; 11/938,096; 11/981,804; 11/981,797; 11/981,793; 11/981,767; 11/981,761; 11/981,730; 11/981,685; 11/981,661; 11/981,607; 11/981,605; 11/927,388; 11/927,356; 11/679,124; 11/541,225; 10/547,214; 11/451,692; and 11/451,691, all of which are incorporated herein by reference in their entirety for all purposes and in particular for all teachings related to forming arrays of DNBs.

In some embodiments, patterned substrates with two dimensional arrays of spots are used to produce arrays of DNBs. The spots are activated to capture and hold the DNBs, while the DNBs do not remain in the areas between spots. In general, a DNB on a spot will repel other DNBs, resulting in one DNB per spot. Since DNBs are three-dimensional (i.e., are not linear short pieces of DNA), arrays of the invention result in more DNA copies per square nanometer of binding surface than traditional DNA arrays. This three-dimensional quality further reduces the quantity of sequencing reagents required, resulting in brighter spots and more efficient imaging. Occupancy of DNB arrays often exceed 90%, but can range from 50% to 100% occupancy.

In further embodiments, the patterned surfaces are produced using standard silicon processing techniques. Such patterned arrays achieve a higher density of DNBs than unpatterned arrays, leading to fewer pixels per base read, faster processing, and increased efficiency in reagent use. In still further embodiments, patterned substrates are 25 mm×75 mm (1″×3″) standard microscope slides, each with the capacity to hold approximately 1 billion individual spots that can bind DNBs. As will be appreciated, slides with even higher densities are encompassed by the present invention. Since DNBs are disposed on a surface and then stick to the activated spots in these embodiments, a high-density DNB array essentially “self-assembles” from DN Bs in solution, eliminating one of the most costly aspects of producing traditional patterned oligo or DNA arrays.

In some embodiments, a surface may have reactive functionalities that react with complementary functionalities on the polynucleotide molecules to form a covalent linkage, e.g., by way of the same techniques used to attach cDNAs to microarrays, e.g., Smirnov et al (2004), Genes, Chromosomes & Cancer, 40: 72-77; Beaucage (2001), Current Medicinal Chemistry, 8: 1213-1244, which are incorporated herein by reference. DNBs may also be efficiently attached to hydrophobic surfaces, such as a clean glass surface that has a low concentration of various reactive functionalities, such as —OH groups. Attachment through covalent bonds formed between the polynucleotide molecules and reactive functionalities on the surface is also referred to herein as “chemical attachment”.

In still further embodiments, polynucleotide molecules can adsorb to a surface. In such an embodiment, the polynucleotide molecules are immobilized through non-specific interactions with the surface, or through non-covalent interactions such as hydrogen bonding, van der Waals forces, and the like.

Attachment may also include wash steps of varying stringencies to remove incompletely attached single molecules or other reagents present from earlier preparation steps whose presence is undesirable or that are nonspecifically bound to surface.

In one aspect, DNBs on a surface are confined to an area of a discrete region. Discrete regions may be incorporated into a surface using methods known in the art and described further herein. In exemplary embodiments, discrete regions contain reactive functionalities or capture probes which can be used to immobilize the polynucleotide molecules.

The discrete regions may have defined locations in a regular array, which may correspond to a rectilinear pattern, hexagonal pattern, or the like. A regular array of such regions is advantageous for detection and data analysis of signals collected from the arrays during an analysis. Also, first- and/or second-stage amplicons confined to the restricted area of a discrete region provide a more concentrated or intense signal, particularly when fluorescent probes are used in analytical operations, thereby providing higher signal-to-noise values. In some embodiments, DNBs are randomly distributed on the discrete regions so that a given region is equally likely to receive any of the different single molecules. In other words, the resulting arrays are not spatially addressable immediately upon fabrication, but may be made so by carrying out an identification, sequencing and/or decoding operation. As such, the identities of the polynucleotide molecules of the invention disposed on a surface are discernable, but not initially known upon their disposition on the surface. In some embodiments, the area of discrete is selected, along with attachment chemistries, macromolecular structures employed, and the like, to correspond to the size of single molecules of the invention so that when single molecules are applied to surface substantially every region is occupied by no more than one single molecule. In some embodiments, DNBs are disposed on a surface comprising discrete regions in a patterned manner, such that specific DNBs (identified, in an exemplary embodiment, by tag adaptors or other labels) are disposed on specific discrete regions or groups of discrete regions.

In some embodiments, the area of discrete regions is less than 1 μm²; and in some embodiments, the area of discrete regions is in the range of from 0.04 μm² to 1 μm²; and in some embodiments, the area of discrete regions is in the range of from 0.2 μm² to 1 μm². In embodiments in which discrete regions are approximately circular or square in shape so that their sizes can be indicated by a single linear dimension, the size of such regions are in the range of from 125 nm to 250 nm, or in the range of from 200 nm to 500 nm. In some embodiments, center-to-center distances of nearest neighbors of discrete regions are in the range of from 0.25 μm to 20 μm; and in some embodiments, such distances are in the range of from 1 μm to 10 μm, or in the range from 50 to 1000 nm. Generally, discrete regions are designed such that a majority of the discrete regions on a surface are optically resolvable. In some embodiments, regions may be arranged on a surface in virtually any pattern in which regions have defined locations.

In further embodiments, molecules are directed to the discrete regions of a surface, because the areas between the discrete regions, referred to herein as “inter-regional areas,” are inert, in the sense that concatemers, or other macromolecular structures, do not bind to such regions. In some embodiments, such inter-regional areas may be treated with blocking agents, e.g., DNAs unrelated to concatemer DNA, other polymers, and the like.

A wide variety of supports may be used with the compositions and methods of the invention to form random arrays. In one aspect, supports are rigid solids that have a surface, preferably a substantially planar surface so that single molecules to be interrogated are in the same plane. The latter feature permits efficient signal collection by detection optics, for example. In another aspect, the support comprises beads, wherein the surface of the beads comprise reactive functionalities or capture probes that can be used to immobilize polynucleotide molecules.

In still another aspect, solid supports of the invention are nonporous, particularly when random arrays of single molecules are analyzed by hybridization reactions requiring small volumes. Suitable solid support materials include materials such as glass, polyacrylamide-coated glass, ceramics, silica, silicon, quartz, various plastics, and the like. In one aspect, the area of a planar surface may be in the range of from 0.5 to 4 cm². In one aspect, the solid support is glass or quartz, such as a microscope slide, having a surface that is uniformly silanized. This may be accomplished using conventional protocols, e.g., acid treatment followed by immersion in a solution of 3-glycidoxypropyl trimethoxysilane, N,N-diisopropylethylamine, and anhydrous xylene (8:1:24 v/v) at 80° C., which forms an epoxysilanized surface. e.g., Beattie et a (1995), Molecular Biotechnology, 4: 213. Such a surface is readily treated to permit end-attachment of capture oligonucleotides, e.g., by providing capture oligonucleotides with a 3′ or 5′ triethylene glycol phosphoryl spacer (see Beattie et al, cited above) prior to application to the surface. Further embodiments for functionalizing and further preparing surfaces for use in the present invention are described for example in U.S. Patent Application Ser. Nos. 60/992,485; 61/026,337; 61/035,914; 61/061,134; 61/116,193; 61/102,586; 12/265,593; 12/266,385; 11/938,096; 11/981,804; 11/981,797; 11/981,793; 11/981,767; 11/981,761; 11/981,730; 11/981,685; 11/981,661; 11/981,607; 11/981,605; 11/927,388; 11/927,356; 11/679,124; 11/541,225; 10/547,214; 11/451,692; and 11/451,691, each of which is herein incorporated by reference in its entirety for all purposes and in particular for all teachings related to preparing surfaces for forming arrays and for all teachings related to forming arrays, particularly arrays of DNBs.

In embodiments of the invention in which patterns of discrete regions are required, photolithography, electron beam lithography, nano imprint lithography, and nano printing may be used to generate such patterns on a wide variety of surfaces, e.g., Pirrung et al, U.S. Pat. No. 5,143,854; Fodor et al, U.S. Pat. No. 5,774,305; Guo, (2004) Journal of Physics D: Applied Physics, 37: R123-141; which are incorporated herein by reference.

As will be appreciated, a wide range of densities of DNBs and/or nucleic acid templates of the invention can be placed on a surface comprising discrete regions to form an array. In some embodiments, each discrete region may comprise from about 1 to about 1000 molecules. In further embodiments, each discrete region may comprise from about 10 to about 900, about 20 to about 800, about 30 to about 700, about 40 to about 600, about 50 to about 500, about 60 to about 400, about 70 to about 300, about 80 to about 200, and about 90 to about 100 molecules.

In some embodiments, arrays of nucleic acid templates and/or DNBs are provided in densities of at least 0.5, 1, 2, 3, 4, 5, 6, 7, 8, 9, or 10 million molecules per square millimeter.

### Methods of Using DNBs

DNBs made according to the methods described above offer an advantage in identifying sequences in target nucleic acids, because the adaptors contained in the DNBs provide points of known sequence that allow spatial orientation and sequence determination when combined with methods utilizing anchors and sequencing probes. In addition, DNBs avoid the cost and challenges of relying on single fluorophore measurements used by single-molecule sequencing systems, because multiple copies of the target sequence are present within a single DNB.

Methods of using DNBs in accordance with the present invention include sequencing and detecting specific sequences in target nucleic acids (e.g., detecting particular target sequences (e.g. specific genes) and/or identifying and/or detecting SNPs). The methods described herein can also be used to detect nucleic acid rearrangements and copy number variation. Nucleic acid quantification, such as digital gene expression (i.e., analysis of an entire transcriptome—all mRNA present in a sample) and detection of the number of specific sequences or groups of sequences in a sample, can also be accomplished using the methods described herein. Although the majority of the discussion herein is directed to identifying sequences of DNBs, it will be appreciated that other, non-concatemeric nucleic acid constructs comprising adaptors may also be used in the embodiments described herein.

Overview of cPAL Sequencing

Sequences of DNBs are generally identified in accordance with the present invention using methods referred to herein as combinatorial probe-anchor ligation (“cPAL”) and variations thereof, as described below. In brief, cPAL involves identifying a nucleotide at a particular detection position in a target nucleic acid by detecting a ligation product formed by ligation of at least one anchor that hybridizes to all or part of an adaptor and a sequencing probe that contains a particular nucleotide at an “interrogation position” that corresponds to (e.g. will hybridize to) the detection position. The sequencing probe contains a unique identifying label. If the nucleotide at the interrogation position is complementary to the nucleotide at the detection position, ligation can occur, resulting in a ligation product containing the unique label which is then detected. Descriptions of different exemplary embodiments of cPAL methods are provided below. It will be appreciated that the following descriptions are not meant to be limiting and that variations of the following embodiments are encompassed by the present invention.

cPAL methods of the present invention have many of the advantages of sequencing by hybridization methods known in the art, including DNA array parallelism, independent and non-iterative base reading, and the capacity to read multiple bases per reaction. In addition, cPAL resolves two limitations of sequencing by hybridization methods: the inability to read simple repeats, and the need for intensive computation.

“Complementary” or “substantially complementary” refers to the hybridization or base pairing or the formation of a duplex between nucleotides or nucleic acids, such as, for instance, between the two strands of a double-stranded DNA molecule or between an oligonucleotide primer and a primer binding site on a single-stranded nucleic acid. Complementary nucleotides are, generally, A and T (or A and U), or C and G. Two single-stranded RNA or DNA molecules are said to be substantially complementary when the nucleotides of one strand, optimally aligned and compared and with appropriate nucleotide insertions or deletions, pair with at least about 80% of the other strand, usually at least about 90% to about 95%, and even about 98% to about 100%.

As used herein, “hybridization” refers to the process in which two single-stranded polynucleotides bind non-covalently to form a stable double-stranded polynucleotide. The resulting (usually) double-stranded polynucleotide is a “hybrid” or “duplex.” “Hybridization conditions” will typically include salt concentrations of less than about 1 M, more usually less than about 500 mM and may be less than about 200 mM. A “hybridization buffer” is a buffered salt solution such as 5% SSPE, or other such buffers known in the art. Hybridization temperatures can be as low as 5° C., but are typically greater than 22° C., and more typically greater than about 30° C., and typically in excess of 37° C. Hybridizations are usually performed under stringent conditions, i.e., conditions under which a probe will hybridize to its target subsequence but will not hybridize to the other, uncomplimentary sequences. Stringent conditions are sequence-dependent and are different in different circumstances. For example, longer fragments may require higher hybridization temperatures for specific hybridization than short fragments. As other factors may affect the stringency of hybridization, including base composition and length of the complementary strands, presence of organic solvents, and the extent of base mismatching, the combination of parameters is more important than the absolute measure of any one parameter alone. Generally stringent conditions are selected to be about 5° C. lower than the Tₘ for the specific sequence at a defined ionic strength and pH. Exemplary stringent conditions include a salt concentration of at least 0.01 M to no more than 1M sodium ion concentration (or other salt) at a pH of about 7.0 to about 8.3 and a temperature of at least 25° C. For example, conditions of 5×SSPE (750 mM NaCl, 50 mM sodium phosphate, 5 mM EDTA at pH 7.4) and a temperature of 30° C. are suitable for allele-specific probe hybridizations. Further examples of stringent conditions are well known in the art, see for example Sambrook J et al. (2001), Molecular Cloning, A Laboratory Manual, (3rd Ed., Cold Spring Harbor Laboratory Press.

As used herein, the term “Tₘ” generally refers to the temperature at which half of the population of double-stranded nucleic acid molecules becomes dissociated into single strands. The equation for calculating the Tm of nucleic acids is well known in the art. As indicated by standard references, a simple estimate of the Tₘ value may be calculated by the equation: Tₘ=81.5+16.6(log 10[Na+])0.41(%[G+C])−675/n−1.0 m, when a nucleic acid is in aqueous solution having cation concentrations of 0.5 M, or less, the (G+C) content is between 30% and 70%, n is the number of bases, and m is the percentage of base pair mismatches (see e.g., Sambrook J et al. (2001), Molecular Cloning, A Laboratory Manual, (3rd Ed., Cold Spring Harbor Laboratory Press). Other references include more sophisticated computations, which take structural as well as sequence characteristics into account for the calculation of Tₘ (see also, Anderson and Young (1985), Quantitative Filter Hybridization, Nucleic Acid Hybridization, and Allawi and SantaLucia (1997), Biochemistry 36:10581-94).

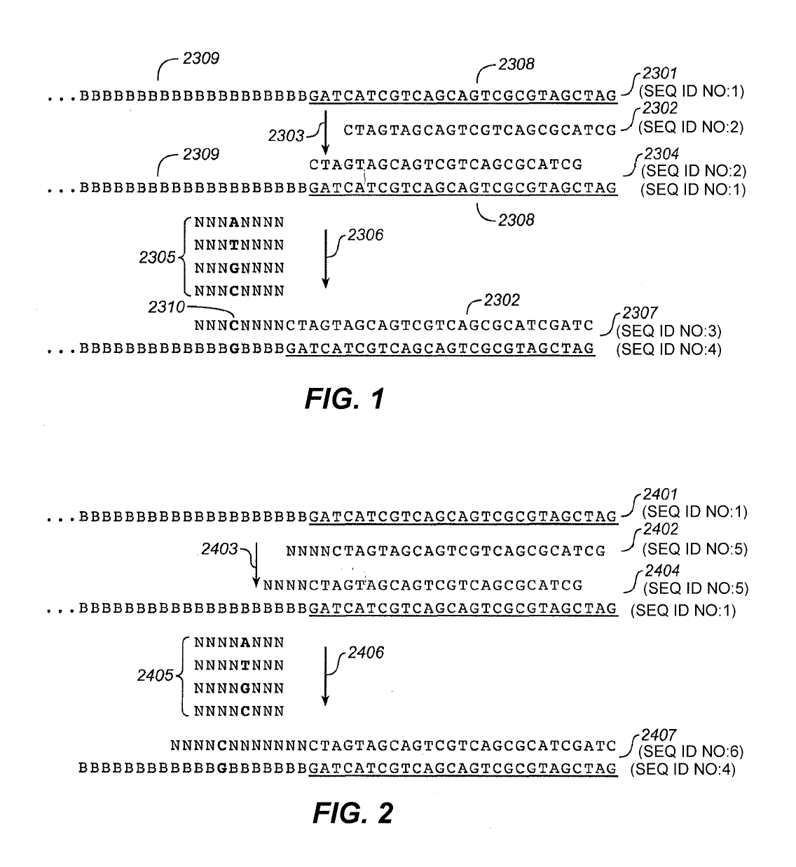

In one example of a cPAL method, referred to herein as “single cPAL”, as illustrated in FIG. 1, anchor 2302 hybridizes to a complementary region on adaptor 2308 of the DNB 2301. Anchor 2302 hybridizes to the adaptor region directly adjacent to target nucleic acid 2309, but in some cases, anchors can be designed to “reach into” the target nucleic acid by incorporating a desired number of degenerate bases at the terminus of the anchor, as is schematically illustrated in FIG. 2 and described further below. A pool of differentially labeled sequencing probes 2305 will hybridize to complementary regions of the target nucleic acid, and sequencing probes that hybridize adjacent to anchors are ligated to the anchors to form a probe ligation product, usually by application of a ligase. The sequencing probes are generally sets or pools of oligonucleotides comprising two parts: different nucleotides at the interrogation position, and then all possible bases (or a universal base) at the other positions; thus, each probe represents each base type at a specific position. The sequencing probes are labeled with a detectable label that differentiates each sequencing probe from the sequencing probes with other nucleotides at that position. Thus, in the example illustrated in FIG. 1, a sequencing probe 2310 that hybridizes adjacent to anchor 2302 and is ligated to the anchor will identify the base at a position in the target nucleic acid five bases from the adaptor as a “G”. FIG. 1 depicts a situation where the interrogation base is five bases in from the ligation site, but as more fully described below, the interrogation base can also be “closer” to the ligation site, and in some cases at the point of ligation. Once ligated, non-ligated anchor and sequencing probes are washed away, and the presence of the ligation product on the array is detected using the label. Multiple cycles of anchor and sequencing probe hybridization and ligation can be used to identify a desired number of bases of the target nucleic acid on each side of each adaptor in a DNB. Hybridization of the anchor and the sequencing probe may occur sequentially or simultaneously. The fidelity of the base call relies in part on the fidelity of the ligase, which generally will not ligate if there is a mismatch close to the ligation site.

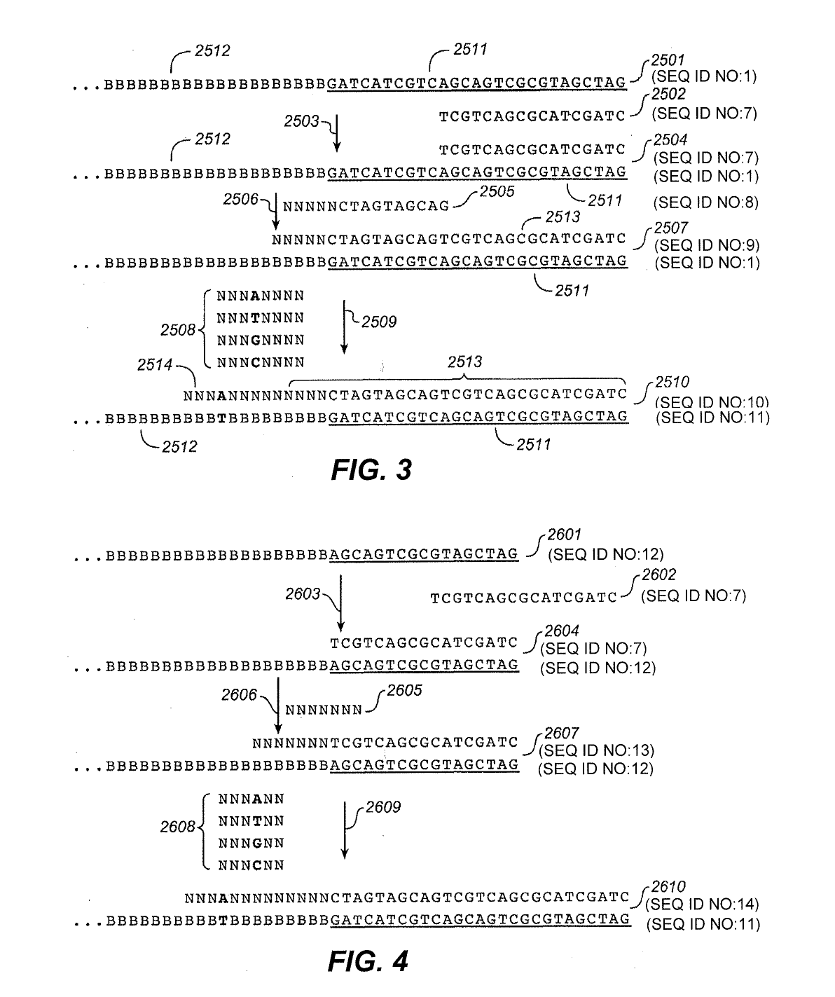

The present invention also provides methods in which two or more anchors are used in every hybridization-ligation cycle. FIG. 3 illustrates an additional example of a “double cPAL with overhang” method in which a first anchor 2502 and a second anchor 2505 each hybridize to complimentary regions of an adaptor. In the example illustrated in FIG. 3, the first anchor 2502 is fully complementary to a first region of the adaptor 2511, and the second anchor 2505 is complementary to a second adaptor region adjacent to the hybridization position of the first anchor. The second anchor also comprises degenerate bases at the terminus that is not adjacent to the first anchor. As a result, the second anchor is able to hybridize to a region of the target nucleic acid 2512 adjacent to adaptor 2511 (the “overhang” portion). The second anchor is generally too short to be maintained alone in its duplex hybridization state, but upon ligation to the first anchor it forms a longer anchor that is stably hybridized for subsequent methods. As discussed above for the “single cPAL” method, a pool of sequencing probes 2508 that represents each base type at a detection position of the target nucleic acid and labeled with a detectable label that differentiates each sequencing probe from the sequencing probes with other nucleotides at that position is hybridized 2509 to the adaptor-anchor duplex and ligated to the terminal 5′ or 3′ base of the anchors. In the example illustrated in FIG. 3, the sequencing probes are designed to interrogate the base that is five positions 5′ of the ligation point between the sequencing probe 2514 and the ligated anchors 2513. Since the second (or “extension”) anchor 2505 has five degenerate bases at its 5′ end, it reaches five bases into the target nucleic acid 2512, allowing interrogation with the sequencing probe at a full ten bases from the interface between the target nucleic acid 2512 and the adaptor 2511.

In double cPAL methods, the bases immediately adjacent an adaptor, which are sequenced using a single anchor (i.e., without one or more extension anchors), are referred to as the “inner positions.” Bases that are five bases further out from the “inner positions” are sequenced using both an anchor and an extension anchor and are referred to as the “outer positions” or the “outer five.” Two, three or more extension anchors can be used to sequence further into the sequence adjacent the adaptor. Extension anchors commonly are fully degenerate (and hybridize to unknown sequence within the target sequence adjacent an adaptor; for that reason they may be referred to as “degenerate anchors.” Therefore, according to one embodiment, an “extension anchor” is actually a pool of random oligomers of a specified length.

In variations of the above described examples of a double cPAL method, if the first anchor terminates closer to the end of the adaptor, the degenerate anchor will be proportionately more degenerate and therefore will have a greater potential to not only ligate to the end of the first anchor but also to ligate to other degenerate anchors at multiple sites on the DNB. To prevent such ligation artifacts, the degenerate anchors can be selectively activated to engage in ligation to a first anchor or to a sequencing probe. Such activation methods are described in further detail below, and include methods such as selectively modifying the termini of the anchors such that they are able to ligate only to a particular anchor or sequencing probe in a particular orientation with respect to the adaptor.

Similar to the double cPAL method described above, it will be appreciated that cPAL methods utilizing three or more anchors (i.e., a first anchor and two or more degenerate anchors) are also encompassed by the present invention.

In addition, sequencing reactions can be done at one or both of the termini of each adaptor, e.g., the sequencing reactions can be “unidirectional” with detection occurring 3′ or 5′ of the adaptor or the other or the reactions can be “bidirectional” in which bases are detected at detection positions 3′ and 5′ of the adaptor. Bidirectional sequencing reactions can occur simultaneously—i.e., bases on both sides of the adaptor are detected at the same time—or sequentially in any order.

Multiple cycles of cPAL (whether single, double, triple, etc.) will identify multiple bases in the regions of the target nucleic acid adjacent to the adaptors. In brief, the cPAL methods are repeated for interrogation of multiple adjacent bases within a target nucleic acid by cycling anchor hybridization and enzymatic ligation reactions with sequencing probe pools designed to detect nucleotides at varying positions removed from the interface between the adaptor and target nucleic acid. In any given cycle, the sequencing probes used are designed such that the identity of one or more of bases at one or more positions is correlated with the identity of the label attached to that sequencing probe. Once the ligated sequencing probe (and hence the base(s) at the interrogation position(s) is detected, the ligated complex is stripped off of the DNB and a new cycle of adaptor and sequencing probe hybridization and ligation is conducted.

As will be appreciated, DNBs of the invention can be used in other sequencing methods in addition to the cPAL methods described above, including other sequencing by ligation methods as well as other sequencing methods, including without limitation sequencing by hybridization, sequencing by synthesis (including sequencing by primer extension), chained sequencing by ligation of cleavable probes, and the like.

Methods similar to those described above for sequencing can also be used to detect specific sequences in a target nucleic acid, including detection of single nucleotide polymorphisms (SNPs). In such methods, sequencing probes that will hybridize to a particular sequence, such as a sequence containing a SNP, will be applied. Such sequencing probes can be differentially labeled to identify which SNP is present in the target nucleic acid. Anchors can also be used in combination with such sequencing probes to provide further stability and specificity.

Loading DNBs onto Flow Slides and Post-Load Treatment

According to one embodiment, DNBs preps are loaded into flow slides as described in Drmanac et al., Science 327:78-81, 2010. Briefly, slides are loaded by pipetting DNBs on the slide. For example, 2- to 3-fold more DNBs than binding sites can be pipetted onto the slide. Loaded slides are incubated for 2 h at 23° C. in a closed chamber, and rinsed to neutralize pH and remove unbound DNBs.

According to another embodiment, after loading such nucleic acid molecules onto nucleic acid arrays, the nucleic acid molecules are stabilized against chemical and physical degradation during biochemical analysis, including but not limited to nucleic acid sequencing, by a post-arraying treatment.

In order to stabilize the arrayed DNBs against chemical and physical degradation during the sequencing process, the DNBs may be treated after they are contacted with and attach to (i.e., loaded onto) the array. According to one embodiment, the DNBs are coated in a layer of partly denatured protein to improve the stability of the DNB array, which in turn improves the intensity and specificity of the signal resulting from cPAL sequencing reactions (described below). Various proteins, including but not limited to serum albumins such as bovine serum albumin (BSA) and human serum albumin, have properties that are conducive to the protective effect and non-interference in the assay in that they do not interact strongly with nucleic acids but bind irreversibly to the array-binding substrate. These properties depend on a number of physico-chemical properties of the stabilizing coat molecule including electrical charging properties, e.g., isoelectric point, molecular weight, non-reactivity with and the inability to intercalate nucleic acid. Without this coating, during the cPAL sequencing process, the quality of the probed DNB signal intensity and specificity can completely degrade in fewer than 30 probe cycles. With this coating, we have used DNB arrays for more than one hundred cycles and routinely see little or no degradation over 70 cycles.

It has been observed that individual DNBs of the array are subject to some degree of spreading on the surface if exposed to the coating process directly after initial load. The addition of a rinse step and a subsequent wash step causing DNB condensation before coating reduces the amount of spreading and physical interactions between adjacent nucleic acid molecules (e.g., intermingling of DNBs), thereby improving the quality of data produced by biochemical analyses, such as probing the DNBs or performing sequencing reactions. Thus, according to one embodiment, the nucleic acid molecules are coated in a layer of partly denatured protein to improve the stability of the nucleic acid molecule array, which in turn improves the intensity and specificity of the signal resulting from biochemical analysis, such as sequencing reactions involving fluorescent dyes.

Although described in terms of the sequencing of genomic DNA in the form of DNBs, post-load treatment according to the present invention is also useful for improving the stability and reducing the spreading of a range of biological molecules, including but not limited to nucleic acids (single- and double-stranded DNA, RNA, etc.), that are attached to or associated with any type of solid support for a wide range of biochemical analyses, including, for example, nucleic acid hybridization, enzymatic reactions (e.g., using endonucleases [including restriction endonucleases], exonucleases, kinases, phosphatases, ligases, etc.), nucleic acid synthesis, nucleic acid amplification (e.g., by the polymerase chain reaction, rolling circle replication, whole-genome amplification, multiple displacement amplification, etc.), and any other form of biochemical analysis known in the art.

### Pre-Anchor Wash

It has been discovered that certain reagents can improve data quality over the course of sequencing. In particular, according to one embodiment, a “pre-anchor wash,” an aqueous wash solution that includes an effective amount of a weak or dilute acid or a cationic surfactant, is used after attaching a nucleic acid to the surface of a solid support (including without limitation, a DNB array as described herein) and before performing the sequencing reaction in each cycle or in later cycles, or at any other time in the sequencing cycle. Any substance can be used for the pre-anchor wash that improves such metrics without interfering with enzymatic reactions in subsequent sequencing steps. Such a pre-anchor wash improves discordance, mappable yield and other metrics of nucleic acid sequencing reactions. Although referred to herein as a “pre-anchor wash,” this wash step may occur at any stage of the sequencing cycle, including without limitation after the strip reagent, after the anchor hybridization or ligation, after the pre-kinase wash, or after the kinase step.

Various treatments were tested in order to reduce the decay of quality of data from cPAL sequencing reactions over 70 cycles, which was observed beginning around cycle 30 to 40. In the standard sequencing protocol, the inside positions are sequenced after the inside positions. As used herein with reference to “double cPAL,” the term “inside positions” refers to the five bases immediately adjacent an adaptor; therefore, the inside positions can be sequenced using an anchor and a probe. The term “outside positions” refers to the next five bases, which can be sequenced using an anchor, a degenerate anchor (which permits sequencing to be performed farther out from the adaptor), and a probe.

Cationic surfactants include but are not limited to benzalkonium chloride, benzethonium chloride, Bronidox, cetyltrimethylammonium bromide (CTAB), cetrimonium chloride, dimethyldioctadecylammonium chloride, lauryl methyl gluceth-10 hydroxypropyl dimonium chloride, and tetramethylammonium hydroxide.

Weak acids include but are not limited to citric acid (Kₐ=1.7×10⁻⁴), nitrous acid (Kₐ=4.6×10⁻⁴, hydrofluoric acid (Kₐ=3.5×10⁻⁴), formic acid (Kₐ=1.8×10⁻⁴), benzoic acid (Kₐ=6.5×10⁻⁵), acetic acid (Kₐ=1.8×10⁻⁵), etc. Citric acid has been shown to perform well in improving data quality over a full 70 cycles of sequencing by the cPAL sequencing method, despite the fact that acidic conditions can cause depurination of the DNA template (partial depurination with 0.25 N hydrochloric acid is commonly used in Southern blotting to promote DNA transfer). In addition to weak acids, a dilute acid of any strength (i.e., Kₐ may be used). Acids with higher Kₐ values, including without limitation strong acids at low concentrations (e.g., less than 5 millimolar), may also be effective in creating the low pH environment that can facilitate the quality improvement.

In the tests described in the Examples, when used on inside positions, a pre-anchor wash was found to reduce discordance by over 40 percent and increase mappable yield by 5 percent, and when used on outside positions, a pre-anchor wash reduced discordance by over 15 percent and increase mappable yield by over 2 percent. In these examples the pre-anchor wash was only used on either the inside or outside positions, although it may be used in each cycle, that is, for both inside and outside positions. According to one embodiment, the pre-anchor wash is used for all cycles, but it can be used for a subset of cycles for example, either the inside or outside positions alone or only after a selected number of cycles (for inside positions, outside positions, or both), e.g., after 10, 20, 30, 40, 50 or 60 cycles.

An effective amount of an acid or cationic surfactant is that amount that reduces discordance or increases mappable yield by a detectable level. According to one embodiment, a pre-anchor wash comprises an amount of an acid or cationic surfactant that reduces discordance by 5, 10, 15, 20, 25, 30, 35, or 40 percent or more at least one position, or increases mappable yield by 0.5, 1.0, 1.5, 2, 3, 4 or 5 percent or more at least one position, or both reduces discordance and increases mappable yield compared to a suitable control.

Sequencing

In one aspect, the present invention provides methods for identifying sequences of DNBs by utilizing sequencing-by-ligation methods. In one aspect, the present invention provides methods for identifying sequences of DNBs that utilize a combinatorial probe-anchor ligation (cPAL) method. Generally, cPAL involves identifying a nucleotide at a detection position in a target nucleic acid by detecting a probe ligation product formed by ligation of an anchor and a sequencing probe. Methods of the invention can be used to sequence a portion or the entire sequence of the target nucleic acid contained in a DNB, and many DNBs that represent a portion or all of a genome.

In some aspects, the ligation reactions in cPAL methods according to the present invention are only driven to about 20% completion. By being “driven to” a specific level of completion as used herein refers to the percentage of individual DNBs or monomers within DNBs that must show a ligation event. Since each base read in a cPAL method is an independent event, every base in every monomer of every DNB does not have to support a ligation reaction in order to be able to read the next bases along the sequence in subsequent hybridization ligation cycles. As a result, cPAL methods of the present invention require dramatically lower amounts of reagents and time, resulting in significant decreases in costs and increases in efficiency. In some embodiments, the ligation reactions in cPAL methods according to the present invention are driven to about 20%, 25%, 30%, 35%, 40%, 45%, 50%, 60%, 70%, 80%, 90% or 100% completion. In further embodiments, ligation reactions in cPAL methods according to the present invention are driven to about 10% to about 100% completion. In still further embodiments, ligation reactions according to the present invention are driven to about 20%-95%, 30%-90%, 40%-85%, 50%-80% and 60%-75% completion. In some embodiments, the percent completion of a reaction is affected by altering reagent concentrations, temperature, and the length of time the reaction is allowed to run. In further embodiments, the percent completion of a cPAL ligation reaction can be estimated by comparing the signal obtained from each DNB in a cPAL ligation reaction and comparing those signals to signals from labeled probes directly hybridized to the anchor hybridization sites of the adaptors in the DNBs. The signal from the labeled probes directly hybridized to the adaptors would provide an estimate of the number of DNBs with available hybridization sites, and this signal could then serve as a baseline to compare to the signals from the ligated probes in a cPAL reaction to determine the percent completion of the ligation reaction. In some embodiments, the completion rate for the ligation reactions may be altered depending on the end use of the information, with some applications desiring a higher level of completion than others.

As discussed further herein, every DNB comprises repeating monomeric units, each monomeric unit comprising one or more adaptors and a target nucleic acid. The target nucleic acid comprises a plurality of detection positions. The term “detection position” refers to a position in a target sequence for which sequence information is desired. As will be appreciated by those in the art, generally a target sequence has multiple detection positions for which sequence information is required, for example in the sequencing of complete genomes as described herein. In some cases, for example in SNP analysis, it may be desirable to just read a single SNP in a particular area.

The present invention provides methods of sequencing that utilize a combination of anchors and sequencing probes. By “sequencing probe” as used herein is meant an oligonucleotide that is designed to provide the identity of a nucleotide at a particular detection position of a target nucleic acid. Sequencing probes hybridize to domains within target sequences, e.g. a first sequencing probe may hybridize to a first target domain, and a second sequencing probe may hybridize to a second target domain. The terms “first target domain” and “second target domain” or grammatical equivalents herein means two portions of a target sequence within a nucleic acid which is under examination. The first target domain may be directly adjacent to the second target domain, or the first and second target domains may be separated by an intervening sequence, for example an adaptor. The terms “first” and “second” are not meant to confer an orientation of the sequences with respect to the 5′-3′ orientation of the target sequence. For example, assuming a 5′-3′ orientation of the complementary target sequence, the first target domain may be located either 5′ to the second domain, or 3′ to the second domain. Sequencing probes can overlap, e.g. a first sequencing probe can hybridize to the first 6 bases adjacent to one terminus of an adaptor, and a second sequencing probe can hybridize to the 4rd-9th bases from the terminus of the adaptor (for example when an anchor has three degenerate bases). Alternatively, a first sequencing probe can hybridize to the 6 bases adjacent to the “upstream” terminus of an adaptor and a second sequencing probe can hybridize to the 6 bases adjacent to the “downstream” terminus of an adaptor.

Sequencing probes will generally comprise a number of degenerate bases and a specific nucleotide at a specific location within the probe to query the detection position (also referred to herein as an “interrogation position”).

In general, pools of sequencing probes are used when degenerate bases are used. That is, a probe having the sequence “NNNANN” is actually a set of probes of having all possible combinations of the four nucleotide bases at five positions (i.e., 1024 sequences) with an adenosine at the 6th position. (As noted herein, this terminology is also applicable to degenerate anchors: for example, when a degenerate anchor has “three degenerate bases”, for example, it is actually a set of oligonucleotides comprising the sequence complementary to the adaptor sequence plus all possible combinations at three positions, so it is a pool of 64 probes).

In some embodiments, for each interrogation position, four differently labeled pools can be combined in a single pool and used in a sequencing step. Thus, in any particular sequencing step, 4 pools are used, each with a different specific base at the interrogation position and with a different label corresponding to the base at the interrogation position. That is, sequencing probes are also generally labeled such that a particular nucleotide at a particular interrogation position is associated with a label that is different from the labels of sequencing probes with a different nucleotide at the same interrogation position. For example, four pools can be used: NNNANN-dye1, NNNTNN-dye2, NNNCNN-dye3 and NNNGNN-dye4 in a single step, as long as the dyes are optically resolvable. In some embodiments, for example for SNP detection, it may only be necessary to include two pools, as the SNP call will be either a C or an A, etc. Similarly, some SNPs have three possibilities. Alternatively, in some embodiments, if the reactions are done sequentially rather than simultaneously, the same dye can be done, just in different steps: e.g. the NNNANN-dye1 probe can be used alone in a reaction, and either a signal is detected or not, and the probes washed away; then a second pool, NNNTNN-dye1 can be introduced.

In any of the sequencing methods described herein, sequencing probes may have a wide range of lengths, including about 3 to about 25 bases. In further embodiments, sequencing probes may have lengths in the range of about 5 to about 20, about 6 to about 18, about 7 to about 16, about 8 to about 14, about 9 to about 12, and about 10 to about 11 bases.

Sequencing probes of the present invention are designed to be complementary, and in general, perfectly complementary, to a sequence of the target sequence such that hybridization of a portion target sequence and probes of the present invention occurs. In particular, it is important that the interrogation position base and the detection position base be perfectly complementary and that the methods of the invention do not result in signals unless this is true.

In many embodiments, sequencing probes are perfectly complementary to the target sequence to which they hybridize; that is, the experiments are run under conditions that favor the formation of perfect basepairing, as is known in the art. As will be appreciated by those in the art, a sequencing probe that is perfectly complementary to a first domain of the target sequence could be only substantially complementary to a second domain of the same target sequence; that is, the present invention relies in many cases on the use of sets of probes, for example, sets of hexamers, that will be perfectly complementary to some target sequences and not to others.

In some embodiments, depending on the application, the complementarity between the sequencing probe and the target need not be perfect; there may be any number of base pair mismatches, which will interfere with hybridization between the target sequence and the single stranded nucleic acids of the present invention. However, if the number of mismatches is so great that no hybridization can occur under even the least stringent of hybridization conditions, the sequence is not a complementary target sequence. Thus, by “substantially complementary” herein is meant that the sequencing probes are sufficiently complementary to the target sequences to hybridize under normal reaction conditions. However, for most applications, the conditions are set to favor probe hybridization only if perfectly complementarity exists. Alternatively, sufficient complementarity is required to allow the ligase reaction to occur; that is, there may be mismatches in some part of the sequence but the interrogation position base should allow ligation only if perfect complementarity at that position occurs.

In some cases, in addition to or instead of using degenerate bases in probes of the invention, universal bases which hybridize to more than one base can be used. For example, inosine can be used. Any combination of these systems and probe components can be utilized.

Sequencing probes of use in methods of the present invention are usually detectably labeled. By “label” or “labeled” herein is meant that a compound has at least one element, isotope or chemical compound attached to enable the detection of the compound. In general, labels of use in the invention include without limitation isotopic labels, which may be radioactive or heavy isotopes, magnetic labels, electrical labels, thermal labels, colored and luminescent dyes, enzymes and magnetic particles as well. Dyes of use in the invention may be chromophores, phosphors or fluorescent dyes, which due to their strong signals provide a good signal-to-noise ratio for decoding. Sequencing probes may also be labeled with quantum dots, fluorescent nanobeads or other constructs that comprise more than one molecule of the same fluorophore. Labels comprising multiple molecules of the same fluorophore will generally provide a stronger signal and will be less sensitive to quenching than labels comprising a single molecule of a fluorophore. It will be understood that any discussion herein of a label comprising a fluorophore will apply to labels comprising single and multiple fluorophore molecules.

Many embodiments of the invention include the use of fluorescent labels. Suitable dyes for use in the invention include, but are not limited to, fluorescent lanthanide complexes, including those of Europium and Terbium, fluorescein, rhodamine, tetramethylrhodamine, eosin, erythrosin, coumarin, methyl-coumarins, pyrene, Malacite green, stilbene, Lucifer Yellow, Cascade Blue™, Texas Red, and others described in the 6th Edition of the Molecular Probes Handbook by Richard P. Haugland, hereby expressly incorporated by reference in its entirety for all purposes and in particular for its teachings regarding labels of use in accordance with the present invention. Commercially available fluorescent dyes for use with any nucleotide for incorporation into nucleic acids include, but are not limited to: Cy3, Cy5, (Amersham Biosciences, Piscataway, N.J., USA), fluorescein, tetramethylrhodamine-, Texas Red®, Cascade Blue®, BODIPY® FL-14, BODIPY®R, BODIPY® TR-14, Rhodamine Green™, Oregon Green® 488, BODIPY® 630/650, BODIPY® 650/665-, Alexa Fluor® 488, Alexa Fluor® 532, Alexa Fluor® 568, Alexa Fluor® 594, Alexa Fluor® 546 (Molecular Probes, Inc. Eugene, Oreg., USA), Quasar 570, Quasar 670, Cal Red 610 (BioSearch Technologies, Novato, Ca). Other fluorophores available for post-synthetic attachment include, inter alia, Alexa Fluor® 350, Alexa Fluor® 532, Alexa Fluor® 546, Alexa Fluor® 568, Alexa Fluor® 594, Alexa Fluor® 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethylrhodamine, Texas Red (available from Molecular Probes, Inc., Eugene, Oreg., USA), and Cy2, Cy3.5, Cy5.5, and Cy7 (Amersham Biosciences, Piscataway, N.J. USA, and others). In some embodiments, the labels used include fluoroscein, Cy3, Texas Red, Cy5, Quasar 570, Quasar 670 and Cal Red 610 are used in methods of the present invention.

Labels can be attached to nucleic acids to form the labeled sequencing probes of the present invention using methods known in the art, and to a variety of locations of the nucleosides. For example, attachment can be at either or both termini of the nucleic acid, or at an internal position, or both. For example, attachment of the label may be done on a ribose of the ribose-phosphate backbone at the 2′ or 3′ position (the latter for use with terminal labeling), in one embodiment through an amide or amine linkage. Attachment may also be made via a phosphate of the ribose-phosphate backbone, or to the base of a nucleotide. Labels can be attached to one or both ends of a probe or to any one of the nucleotides along the length of a probe.

Sequencing probes are structured differently depending on the interrogation position desired. For example, in the case of sequencing probes labeled with fluorophores, a single position within each sequencing probe will be correlated with the identity of the fluorophore with which it is labeled. Generally, the fluorophore molecule will be attached to the end of the sequencing probe that is opposite to the end targeted for ligation to the anchor.

By “anchor” as used herein is meant an oligonucleotide designed to be complementary to at least a portion of an adaptor, referred to herein as “an anchor site”. Depending on the context, an “anchor” may function as a primer, as, for example, in sequencing-by-synthesis reactions in which one or more nucleotide bases are added to the end of a primer by a polymerase or other enzyme. Adaptors can contain multiple anchor sites for hybridization with multiple anchors, as described herein. As discussed further herein, anchors of use in the present invention can be designed to hybridize to an adaptor such that at least one end of the anchor is flush with one terminus of the adaptor (either “upstream” or “downstream”, or both). In further embodiments, anchors can be designed to hybridize to at least a portion of an adaptor (a first adaptor site) and also at least one nucleotide of the target nucleic acid adjacent to the adaptor (“overhangs”). As illustrated in FIG. 2, anchor 2402 comprises a sequence complementary to a portion of the adaptor. Anchor 2402 also comprises four degenerate bases at one terminus. This degeneracy allows for a portion of the anchor population to fully or partially match the sequence of the target nucleic acid adjacent to the adaptor and allows the anchor to hybridize to the adaptor and reach into the target nucleic acid adjacent to the adaptor regardless of the identity of the nucleotides of the target nucleic acid adjacent to the adaptor. This shift of the terminal base of the anchor into the target nucleic acid shifts the position of the base to be called closer to the ligation point, thus allowing the fidelity of the ligase to be maintained. In general, ligases ligate probes with higher efficiency if the probes are perfectly complementary to the regions of the target nucleic acid to which they are hybridized, but the fidelity of ligases decreases with distance away from the ligation point. Thus, in order to minimize and/or prevent errors due to incorrect pairing between a sequencing probe and the target nucleic acid, it can be useful to maintain the distance between the nucleotide to be detected and the ligation point of the sequencing and anchors. By designing the anchor to reach into the target nucleic acid, the fidelity of the ligase is maintained while still allowing a greater number of nucleotides adjacent to each adaptor to be identified. Although the embodiment illustrated in FIG. 2 is one in which the sequencing probe hybridizes to a region of the target nucleic acid on one side of the adaptor, it will be appreciated that embodiments in which the sequencing probe hybridizes on the other side of the adaptor are also encompassed by the invention. In FIG. 2, “N” represents a degenerate base and “B” represents nucleotides of undetermined sequence. As will be appreciated, in some embodiments, rather than degenerate bases, universal bases may be used.

Anchors of the invention may comprise any sequence that allows the anchor to hybridize to a DNB, generally to an adaptor of a DNB. Such anchors may comprise a sequence such that when the anchor is hybridized to an adaptor, the entire length of the anchor is contained within the adaptor. In some embodiments, anchors may comprise a sequence that is complementary to at least a portion of an adaptor and also comprise degenerate bases that are able to hybridize to target nucleic acid regions adjacent to the adaptor. In some exemplary embodiments, anchors are hexamers that comprise 3 bases that are complementary to an adaptor and 3 degenerate bases. In some exemplary embodiments, anchors are 8-mers that comprise 3 bases that are complementary to an adaptor and 5 degenerate bases. In further exemplary embodiments, particularly when multiple anchors are used, a first anchor comprises a number of bases complementary to an adaptor at one end and degenerate bases at another end, whereas a second anchor comprises all degenerate bases and is designed to ligate to the end of the first anchor that comprises degenerate bases. It will be appreciated that these are exemplary embodiments, and that a wide range of combinations of known and degenerate bases can be used to produce anchors of use in accordance with the present invention.

The present invention provides sequencing by ligation methods for identifying sequences of DNBs. In certain aspects, the sequencing by ligation methods of the invention include providing different combinations of anchors and sequencing probes, which, when hybridized to adjacent regions on a DNB, can be ligated to form probe ligation products. The probe ligation products are then detected, which provides the identity of one or more nucleotides in the target nucleic acid. By “ligation” as used herein is meant any method of joining two or more nucleotides to each other. Ligation can include chemical as well as enzymatic ligation. In general, the sequencing by ligation methods discussed herein utilize enzymatic ligation by ligases. Such ligases invention can be the same or different than ligases discussed above for creation of the nucleic acid templates. Such ligases include without limitation DNA ligase I, DNA ligase II, DNA ligase III, DNA ligase IV, E. coli DNA ligase, T4 DNA ligase, T4 RNA ligase 1, T4 RNA ligase 2, T7 ligase, T3 DNA ligase, and thermostable ligases (including without limitation Taq ligase) and the like. As discussed above, sequencing by ligation methods often rely on the fidelity of ligases to only join probes that are perfectly complementary to the nucleic acid to which they are hybridized. This fidelity will decrease with increasing distance between a base at a particular position in a probe and the ligation point between the two probes. As such, conventional sequencing by ligation methods can be limited in the number of bases that can be identified. The present invention increases the number of bases that can be identified by using multiple probe pools, as is described further herein.

A variety of hybridization conditions may be used in the sequencing by ligation methods of sequencing as well as other methods of sequencing described herein. These conditions include high, moderate and low stringency conditions; see for example Maniatis et al., Molecular Cloning: A Laboratory Manual, 2d Edition, 1989, and Short Protocols in Molecular Biology, ed. Ausubel, et al, which are hereby incorporated by reference. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Acid Probes, “Overview of principles of hybridization and the strategy of nucleic acid assays,” (1993). Generally, stringent conditions are selected to be about 5-10° C. lower than the thermal melting point (Tm) for the specific sequence at a defined ionic strength and pH. The Tm is the temperature (under defined ionic strength, pH and nucleic acid concentration) at which 50% of the probes complementary to the target hybridize to the target sequence at equilibrium (as the target sequences are present in excess, at Tm, 50% of the probes are occupied at equilibrium). Stringent conditions can be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g. 10 to 50 nucleotides) and at least about 60° C. for long probes (e.g. greater than 50 nucleotides). Stringent conditions may also be achieved with the addition of helix destabilizing agents such as formamide. The hybridization conditions may also vary when a non-ionic backbone, i.e. PNA is used, as is known in the art. In addition, cross-linking agents may be added after target binding to cross-link, i.e. covalently attach, the two strands of the hybridization complex.

Although much of the description of sequencing methods is provided in terms of nucleic acid templates of the invention, it will be appreciated that these sequencing methods also encompass identifying sequences in DNBs generated from such nucleic acid templates, as described herein.

For any of sequencing methods known in the art and described herein using nucleic acid templates of the invention, the present invention provides methods for determining at least about 10 to about 200 bases in target nucleic acids. In further embodiments, the present invention provides methods for determining at least about 20 to about 180, about 30 to about 160, about 40 to about 140, about 50 to about 120, about 60 to about 100, and about 70 to about 80 bases in target nucleic acids. In still further embodiments, sequencing methods are used to identify at least 5, 10, 15, 20, 25, 30 or more bases adjacent to one or both ends of each adaptor in a nucleic acid template of the invention.

Any of the sequencing methods described herein and known in the art can be applied to nucleic acid templates and/or DN Bs of the invention in solution or to nucleic acid templates and/or DNBs disposed on a surface and/or in an array.

Single cPAL

In one aspect, the present invention provides methods for identifying sequences of DNBs by using combinations of sequencing and anchors that hybridize to adjacent regions of a DNB and are ligated, usually by application of a ligase. Such methods are generally referred to herein as cPAL (combinatorial probe anchor ligation) methods. In one aspect, cPAL methods of the invention produce probe ligation products comprising a single anchor and a single sequencing probe. Such cPAL methods in which only a single anchor is used are referred to herein as “single cPAL”.

One embodiment of single cPAL is illustrated in FIG. 1. A monomeric unit 2301 of a DNB comprises a target nucleic acid 2309 and an adaptor 2308. An anchor 2302 hybridizes to a complementary region on adaptor 2308. In the example illustrated in FIG. 1, anchor 2302 hybridizes to the adaptor region directly adjacent to target nucleic acid 2309, although, as is discussed further herein, anchors can also be designed to reach into the target nucleic acid adjacent to an adaptor by incorporating a desired number of degenerate bases at the terminus of the anchor. A pool of differentially labeled sequencing probes 2306 will hybridize to complementary regions of the target nucleic acid. A sequencing probe 2310 that hybridizes to the region of target nucleic acid 2309 adjacent to anchor 2302 will be ligated to the anchor form a probe ligation product. The efficiency of hybridization and ligation is increased when the base in the interrogation position of the probe is complementary to the unknown base in the detection position of the target nucleic acid. This increased efficiency favors ligation of perfectly complementary sequencing probes to anchors over mismatch sequencing probes. As discussed above, ligation is generally accomplished enzymatically using a ligase, but other ligation methods can also be utilized in accordance with the invention. In FIG. 1, “N” represents a degenerate base and “B” represents nucleotides of undetermined sequence. As will be appreciated, in some embodiments, rather than degenerate bases, universal bases may be used.

As also discussed above, the sequencing probes can be oligonucleotides representing each base type at a specific position and labeled with a detectable label that differentiates each sequencing probe from the sequencing probes with other nucleotides at that position. Thus, in the example illustrated in FIG. 1, a sequencing probe 2310 that hybridizes adjacent to anchor 2302 and is ligated to the anchor will identify the base at a position in the target nucleic acid 5 bases from the adaptor as a “G”. Multiple cycles of anchor and sequencing probe hybridization and ligation can be used to identify a desired number of bases of the target nucleic acid on each side of each adaptor in a DNB.

As will be appreciated, hybridization of the anchor and the sequencing probe can be sequential or simultaneous in any of the cPAL methods described herein.

In the embodiment illustrated in FIG. 1, sequencing probe 2310 hybridizes to a region “upstream” of the adaptor, however it will be appreciated that sequencing probes may hybridize either “upstream” or “downstream” of the adaptor to identify nucleotides at positions in the nucleic acid on both sides of the adaptor. Such embodiments allow generation of multiple points of data from each adaptor for each hybridization-ligation-detection cycle of the single cPAL method. The terms “upstream” and “downstream” refer to the regions 5′ and 3′ of the adaptor, depending on the orientation of the system. In general, “upstream” and “downstream” are relative terms and are not meant to be limiting; rather they are used for ease of understanding.

In some embodiments, probes used in a single cPAL method may have from about 3 to about 20 bases corresponding to an adaptor and from about 1 to about 20 degenerate bases (i.e., in a pool of anchors). Such anchors may also include universal bases, as well as combinations of degenerate and universal bases.

In some embodiments, anchors with degenerated bases may have about 1-5 mismatches with respect to the adaptor sequence to increase the stability of full match hybridization at the degenerated bases. Such a design provides an additional way to control the stability of the ligated anchor and sequencing probes to favor those probes that are perfectly matched to the target (unknown) sequence. In further embodiments, a number of bases in the degenerate portion of the anchors may be replaced with basic sites (i.e., sites which do not have a base on the sugar) or other nucleotide analogs to influence the stability of the hybridized probe to favor the full match hybrid at the distal end of the degenerate part of the anchor that will participate in the ligation reactions with the sequencing probes, as described herein. Such modifications may be incorporated, for example, at interior bases, particularly for anchors that comprise a large number (i.e., greater than 5) of degenerated bases. In addition, some of the degenerated or universal bases at the distal end of the anchor may be designed to be cleavable after hybridization (for example by incorporation of a uracil) to generate a ligation site to the sequencing probe or to a second anchor, as described further below.

In further embodiments, the hybridization of the anchors can be controlled through manipulation of the reaction conditions, for example the stringency of hybridization. In an exemplary embodiment, the anchor hybridization process may start with conditions of high stringency (higher temperature, lower salt, higher pH, higher concentration of formamide, and the like), and these conditions may be gradually or stepwise relaxed. This may require consecutive hybridization cycles in which different pools of anchors are removed and then added in subsequent cycles. Such methods provide a higher percentage of target nucleic acid occupied with perfectly complementary anchors, particularly anchors perfectly complementary at positions at the distal end that will be ligated to the sequencing probe. Hybridization time at each stringency condition may also be controlled to obtain greater numbers of full match hybrids.

Double cPAL (and Beyond)

In still further embodiments, the present invention provides cPAL methods utilizing two ligated anchors in every hybridization-ligation cycle. See for example U.S. Patent Application Ser. Nos. 60/992,485; 61/026,337; 61/035,914 and 61/061,134, which are hereby expressly incorporated by reference in their entirety, and especially the examples and claims. FIG. 3 illustrates an example of a “double cPAL” method in which a first anchor 2502 and a second anchor 2505 hybridize to complimentary regions of an adaptor; that is, the first anchor hybridizes to the first anchor site and the second anchor hybridizes to the second adaptor site. In the example illustrated in FIG. 3, the first anchor 2502 is fully complementary to a region of the adaptor 2511 (the first anchor site), and the second anchor 2505 is complementary to the adaptor region adjacent to the hybridization position of the first anchor (the second anchor site). In general, the first and second anchor sites are adjacent.

The second anchor may optionally also comprises degenerate bases at the terminus that is not adjacent to the first anchor such that it will hybridize to a region of the target nucleic acid 2512 adjacent to adaptor 2511. This allows sequence information to be generated for target nucleic acid bases farther away from the adaptor/target interface. Again, as outlined herein, when a probe is said to have “degenerate bases”, it means that the probe actually comprises a set of probes, with all possible combinations of sequences at the degenerate positions. For example, if an anchor is 9 bases long with 6 known bases and three degenerate bases, the anchor is actually a pool of 64 probes.

The second anchor is generally too short to be maintained alone in its duplex hybridization state, but upon ligation to the first anchor it forms a longer anchor that is stable for subsequent methods. In the some embodiments, the second anchor has about 1 to about 5 bases that are complementary to the adaptor and about 5 to about 10 bases of degenerate sequence. As discussed above for the “single cPAL” method, a pool of sequencing probes 2508 representing each base type at a detection position of the target nucleic acid and labeled with a detectable label that differentiates each sequencing probe from the sequencing probes with other nucleotides at that position is hybridized 2509 to the adaptor-anchor duplex and ligated to the terminal 5′ or 3′ base of the ligated anchors. In the example illustrated in FIG. 3, the sequencing probes are designed to interrogate the base that is five positions 5′ of the ligation point between the sequencing probe 2514 and the ligated anchors 2513. Since the second anchor 2505 has five degenerate bases at its 5′ end, it reaches 5 bases into the target nucleic acid 2512, allowing interrogation with the sequencing probe at a full 10 bases from the interface between the target nucleic acid 2512 and the adaptor 2511. In FIG. 3, “N” represents a degenerate base and “B” represents nucleotides of undetermined sequence. As will be appreciated, in some embodiments, rather than degenerate bases, universal bases may be used.

In some embodiments, the second anchor may have about 5-10 bases corresponding to an adaptor and about 5-15 bases, which are generally degenerated, corresponding to the target nucleic acid. This second anchor may be hybridized first under optimal conditions to favor high percentages of target occupied with full match at a few bases around the ligation point between the two anchors. The first anchor and/or the sequencing probe may be hybridized and ligated to the second degenerate anchor in a single step or sequentially. In some embodiments, the first and second anchors may have at their ligation point from about 5 to about 50 complementary bases that are not complementary to the adaptor, thus forming a “branching-out” hybrid. This design allows an adaptor-specific stabilization of the hybridized second anchor. In some embodiments, the second anchor is ligated to the sequencing probe before hybridization of the first anchor; in some embodiments the second anchor is ligated to the first anchor prior to hybridization of the sequencing probe; in some embodiments the first and second anchors and the sequencing probe hybridize simultaneously and ligation occurs between the first and second anchor and between the second anchor and the sequencing probe simultaneously or essentially simultaneously, while in other embodiments the ligation between the first and second anchor and between the second anchor and the sequencing probe occurs sequentially in any order. Stringent washing conditions can be used to remove unligated probes; (e.g., using temperature, pH, salt, a buffer with an optimal concentration of formamide can all be used, with optimal conditions and/or concentrations being determined using methods known in the art). Such methods can be particularly useful in methods utilizing second anchors with large numbers of degenerated bases that are hybridized outside of the corresponding junction point between the anchor and the target nucleic acid.

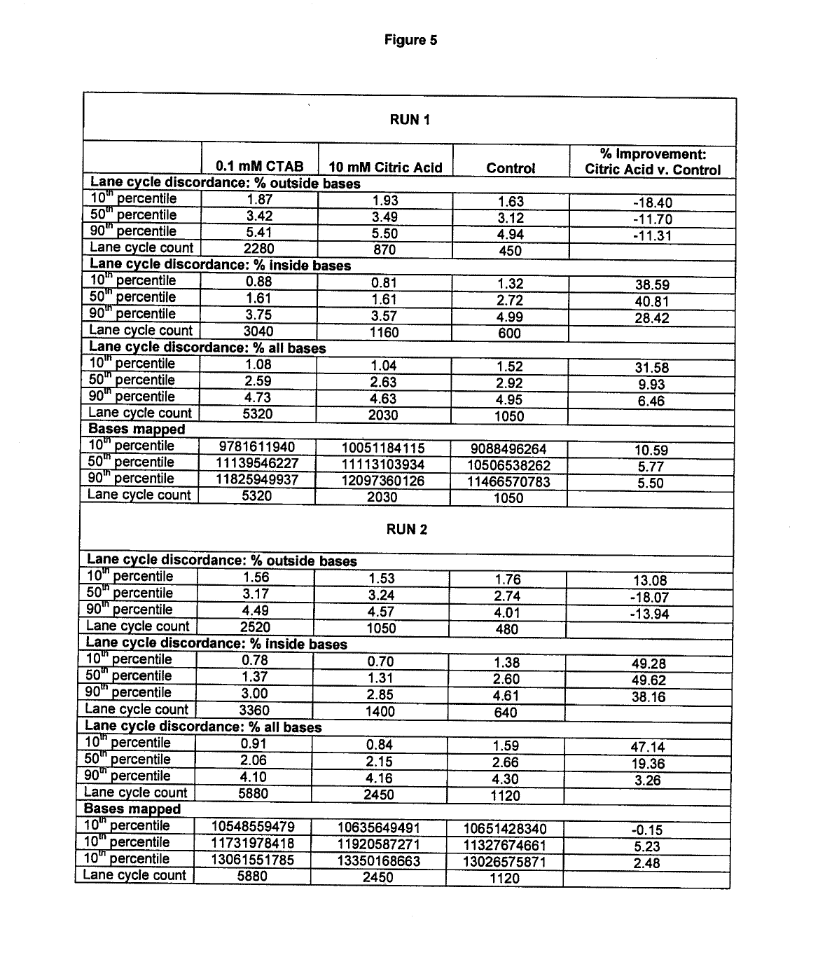

In certain embodiments, double cPAL methods utilize ligation of two anchors in which one anchor is fully complementary to an adaptor and the second anchor is fully degenerate (again, actually a pool of probes). An example of such a double cPAL method is illustrated in FIG. 4, in which the first anchor 2602 is hybridized to adaptor 2611 of DNB 2601. The second anchor 2605 is fully degenerate and is thus able to hybridize to the unknown nucleotides of the region of the target nucleic acid 2612 adjacent to adaptor 2611. The second anchor is designed to be too short to be maintained alone in its duplex hybridization state, but upon ligation to the first anchor the formation of the longer ligated anchor construct provides the stability needed for subsequent steps of the cPAL process. The second fully degenerate anchor may in some embodiments be from about 5 to about 20 bases in length. For longer lengths (i.e., above 10 bases), alterations to hybridization and ligation conditions may be introduced to lower the effective Tm of the degenerate anchor. The shorter second anchor will generally bind non-specifically to target nucleic acid and adaptors, but its shorter length will affect hybridization kinetics such that in general only those second anchors that are perfectly complementary to regions adjacent to the adaptors and the first anchors will have the stability to allow the ligase to join the first and second anchors, generating the longer ligated anchor construct. Non-specifically hybridized second anchors will not have the stability to remain hybridized to the DNB long enough to subsequently be ligated to any adjacently hybridized sequencing probes. In some embodiments, after ligation of the second and first anchors, any unligated anchors will be removed, usually by a wash step. In FIG. 4, “N” represents a degenerate base and “B” represents nucleotides of undetermined sequence. As will be appreciated, in some embodiments, rather than degenerate bases, universal bases may be used.

In further exemplary embodiments, the first anchor will be a hexamer comprising 3 bases complementary to the adaptor and 3 degenerate bases, whereas the second anchor comprises only degenerate bases and the first and second anchors are designed such that only the end of the first anchor with the degenerate bases will ligate to the second anchor. In further exemplary embodiments, the first anchor is an 8-mer comprising 3 bases complementary to an adaptor and 5 degenerate bases, and again the first and second anchors are designed such that only the end of the first anchor with the degenerate bases will ligate to the second anchor. It will be appreciated that these are exemplary embodiments and that a wide range of combinations of known and degenerate bases can be used in the design of both the first and second (and in some embodiments the third and/or fourth) anchors.

In variations of the above described examples of a double cPAL method, if the first anchor terminates closer to the end of the adaptor, the second anchor will be proportionately more degenerate and therefore will have a greater potential to not only ligate to the end of the first anchor but also to ligate to other second anchors at multiple sites on the DNB. To prevent such ligation artifacts, the second anchors can be selectively activated to engage in ligation to a first anchor or to a sequencing probe. Such activation include selectively modifying the termini of the anchors such that they are able to ligate only to a particular anchor or sequencing probe in a particular orientation with respect to the adaptor. For example, 5′ and 3′ phosphate groups can be introduced to the second anchor, with the result that the modified second anchor would be able to ligate to the 3′ end of a first anchor hybridized to an adaptor, but two second anchors would not be able to ligate to each other (because the 3′ ends are phosphorylated, which would prevent enzymatic ligation). Once the first and second anchors are ligated, the 3′ ends of the second anchor can be activated by removing the 3′ phosphate group (for example with T4 polynucleotide kinase or phosphatases such as shrimp alkaline phosphatase and calf intestinal phosphatase).

If it is desired that ligation occur between the 3′ end of the second anchor and the 5′ end of the first anchor, the first anchor can be designed and/or modified to be phosphorylated on its 5′ end and the second anchor can be designed and/or modified to have no 5′ or 3′ phosphorylation. Again, the second anchor would be able to ligate to the first anchor, but not to other second anchors. Following ligation of the first and second anchors, a 5′ phosphate group can be produced on the free terminus of the second anchor (for example, by using T4 polynucleotide kinase) to make it available for ligation to sequencing probes in subsequent steps of the cPAL process.

In some embodiments, the two anchors are applied to the DNBs simultaneously. In some embodiments, the two anchors are applied to the DNBs sequentially, allowing one of the anchors to hybridize to the DNBs before the other. In some embodiments, the two anchors are ligated to each other before the second adaptor is ligated to the sequencing probe. In some embodiments, the anchors and the sequencing probe are ligated in a single step. In embodiments in which two anchors and the sequencing probe are ligated in a single step, the second adaptor can be designed to have enough stability to maintain its position until all three probes (the two anchors and the sequencing probe) are in place for ligation. For example, a second anchor comprising five bases complementary to the adaptor and five degenerate bases for hybridization to the region of the target nucleic acid adjacent to the adaptor can be used. Such a second anchor may have sufficient stability to be maintained with low stringency washing, and thus a ligation step would not be necessary between the steps of hybridization of the second anchor and hybridization of a sequencing probe. In the subsequent ligation of the sequencing probe to the second anchor, the second anchor would also be ligated to the first anchor, resulting in a duplex with increased stability over any of the anchors or sequencing probes alone.

Similar to the double cPAL method described above, it will be appreciated that cPAL with three or more anchors is also encompassed by the present invention. Such anchors can be designed in accordance with methods described herein and known in the art to hybridize to regions of adaptors such that one terminus of one of the anchors is available for ligation to sequencing probes hybridized adjacent to the terminal anchor. In an exemplary embodiment, three anchors are provided—two are complementary to different sequences within an adaptor and the third comprises degenerate bases to hybridize to sequences within the target nucleic acid. In a further embodiment, one of the two anchors complementary to sequences within the adaptor may also comprise one or more degenerate bases at on terminus, allowing that anchor to reach into the target nucleic acid for ligation with the third anchor. In further embodiments, one of the anchors may be fully or partially complementary to the adaptor and the second and third anchors will be fully degenerate for hybridization to the target nucleic acid. Four or more fully degenerate anchors can in further embodiments be ligated sequentially to the three ligated anchors to achieve extension of reads further into the target nucleic acid sequence. In an exemplary embodiment, a first anchor comprising twelve bases complementary to an adaptor may ligate with a second hexameric anchor in which all six bases are degenerate. A third anchor, also a fully degenerate hexamer, can also ligate to the second anchor to further extend into the unknown sequence of the target nucleic acid. A fourth, fifth, sixth, etc. anchor may also be added to extend even further into the unknown sequence. In still further embodiments and in accordance with any of the cPAL methods described herein, one or more of the anchors may comprise one or more labels that serve to “tag” the anchor and/or identify the particular anchor hybridized to an adaptor of a DNB.

Detecting Fluorescently Labeled Sequencing Probes

As discussed above, sequencing probes used in accordance with the present invention may be detectably labeled with a wide variety of labels. Although the following description is primarily directed to embodiments in which the sequencing probes are labeled with fluorophores, it will be appreciated that similar embodiments utilizing sequencing probes comprising other kinds of labels are encompassed by the present invention.

Multiple cycles of cPAL (whether single, double, triple, etc.) will identify multiple bases in the regions of the target nucleic acid adjacent to the adaptors. In brief, the cPAL methods are repeated for interrogation of multiple bases within a target nucleic acid by cycling anchor hybridization and enzymatic ligation reactions with sequencing probe pools designed to detect nucleotides at varying positions removed from the interface between the adaptor and target nucleic acid. In any given cycle, the sequencing probes used are designed such that the identity of one or more of bases at one or more positions is correlated with the identity of the label attached to that sequencing probe. Once the ligated sequencing probe (and hence the base(s) at the interrogation position(s) is detected, the ligated complex is stripped off of the DNB and a new cycle of adaptor and sequencing probe hybridization and ligation is conducted.

In general, four fluorophores are generally used to identify a base at an interrogation position within a sequencing probe, and a single base is queried per hybridization-ligation-detection cycle. However, as will be appreciated, embodiments utilizing 8, 16, 20 and 24 fluorophores or more are also encompassed by the present invention. Increasing the number of fluorophores increases the number of bases that can be identified during any one cycle.

In one exemplary embodiment, a set of 7-mer pools of sequencing probes is employed having the following structures:

3′-F1-NNNNNNAp
3′-F2-NNNNNNGp
3′-F3-NNNNNNCp
3′-F4-NNNNNNTp

The “p” represents a phosphate available for ligation and “N” represents degenerate bases. F1-F4 represent four different fluorophores—each fluorophore is thus associated with a particular base. This exemplary set of probes would allow detection of the base immediately adjacent to the adaptor upon ligation of the sequencing probe to an anchor hybridized to the adaptor. To the extent that the ligase used to ligate the sequencing probe to the anchor discriminates for complementarity between the base at the interrogation position of the probe and the base at the detection position of the target nucleic acid, the fluorescent signal that would be detected upon hybridization and ligation of the sequencing probe provides the identity of the base at the detection position of the target nucleic acid.

In some embodiments, a set of sequencing probes will comprise three differentially labeled sequencing probes, with a fourth optional sequencing probe left unlabeled.

After performing a hybridization-ligation-detection cycle, the anchor-sequencing probe ligation products are stripped and a new cycle is begun. In some embodiments, accurate sequence information can be obtained as far as six bases or more from the ligation point between the anchor and sequencing probes and as far as twelve bases or more from the interface between the target nucleic acid and the adaptor. The number of bases that can be identified can be increased using methods described herein, including the use of anchors with degenerate ends that are able to reach further into the target nucleic acid.

Imaging acquisition may be performed using methods known in the art, including the use of commercial imaging packages such as Metamorph (Molecular Devices, Sunnyvale, Calif.). Data extraction may be performed by a series of binaries written in, e.g., C/C++ and base-calling and read-mapping may be performed by a series of Matlab and Perl scripts.

In an exemplary embodiment, DNBs disposed on a surface undergo a cycle of cPAL as described herein in which the sequencing probes utilized are labeled with four different fluorophores (each corresponding to a particular base at an interrogation position within the probe). To determine the identity of a base of each DNB disposed on the surface, each field of view (“frame”) is imaged with four different wavelengths corresponding the to the four fluorescently labeled sequencing probes. All images from each cycle are saved in a cycle directory, where the number of images is four times the number of frames (when four fluorophores are used). Cycle image data can then be saved into a directory structure organized for downstream processing.

In some embodiments, data extraction will rely on two types of image data: bright-field images to demarcate the positions of all DNBs on a surface, and sets of fluorescence images acquired during each sequencing cycle. Data extraction software can be used to identify all objects with the bright-field images and then for each such object, the software can be used to compute an average fluorescence value for each sequencing cycle. For any given cycle, there are four data points, corresponding to the four images taken at different wavelengths to query whether that base is an A, G, C or T. These raw data points (also referred to herein as “base calls”) are consolidated, yielding a discontinuous sequencing read for each DNB.

The population of identified bases can then be assembled to provide sequence information for the target nucleic acid and/or identify the presence of particular sequences in the target nucleic acid. In some embodiments, the identified bases are assembled into a complete sequence through alignment of overlapping sequences obtained from multiple sequencing cycles performed on multiple DNBs. As used herein, the term “complete sequence” refers to the sequence of partial or whole genomes as well as partial or whole target nucleic acids. In further embodiments, assembly methods utilize algorithms that can be used to “piece together” overlapping sequences to provide a complete sequence. In still further embodiments, reference tables are used to assist in assembling the identified sequences into a complete sequence. A reference table may be compiled using existing sequencing data on the organism of choice. For example human genome data can be accessed through the National Center for Biotechnology Information at ftp.ncbi.nih.gov/refseq/release, or through the J. Craig Venter Institute at http://www.jcvi.org/researchhuref/. All or a subset of human genome information can be used to create a reference table for particular sequencing queries. In addition, specific reference tables can be constructed from empirical data derived from specific populations, including genetic sequence from humans with specific ethnicities, geographic heritage, religious or culturally-defined populations, as the variation within the human genome may slant the reference data depending upon the origin of the information contained therein.

In any of the embodiments of the invention discussed herein, a population of nucleic acid templates and/or DNBs may comprise a number of target nucleic acids to substantially cover a whole genome or a whole target polynucleotide. As used herein, “substantially covers” means that the amount of nucleotides (i.e., target sequences) analyzed contains an equivalent of at least two copies of the target polynucleotide, or in another aspect, at least ten copies, or in another aspect, at least twenty copies, or in another aspect, at least 100 copies. Target polynucleotides may include DNA fragments, including genomic DNA fragments and cDNA fragments, and RNA fragments. Guidance for the step of reconstructing target polynucleotide sequences can be found in the following references, which are incorporated by reference: Lander et al, Genomics, 2: 231-239 (1988); Vingron et al, J. Mol. Biol., 235: 1-12 (1994); and like references.

Sets of Probes

As will be appreciated, different combinations of sequencing and anchors can be used in accordance with the various cPAL methods described above. The following descriptions of sets of probes (also referred to herein as “pools of probes”) of use in the present invention are exemplary embodiments and it will be appreciated that the present invention is not limited to these combinations.

In one aspect, sets of probes are designed for identification of nucleotides at positions at a specific distance from an adaptor. For example, certain sets of probes can be used to identify bases up to 3, 4, 5, 6, 7, 8, 9, 10, 11, 12, 13, 14, 15, 16, 17, 18, 19, 20, 21, 22, 23, 24, 25, 26, 27, 28, 29, 30 and more positions away from the adaptor. As discussed above, anchors with degenerate bases at one terminus can be designed to reach into the target nucleic acid adjacent to an adaptor, allowing sequencing probes to ligate further away from the adaptor and thus provide the identity of a base further away from the adaptor.

In an exemplary embodiment, a set of probes comprises at least two anchors designed to hybridize to adjacent regions of an adaptor. In one embodiment, the first anchor is fully complementary to a region of the adaptor, while the second anchor is complementary to the adjacent region of the adaptor. In some embodiments, the second anchor will comprise one or more degenerate nucleotides that extend into and hybridize to nucleotides of the target nucleic acid adjacent to the adaptor. In an exemplary embodiment, the second anchor comprises at least 1-10 degenerate bases. In a further exemplary embodiment, the second anchor comprises 2-9, 3-8, 4-7, and 5-6 degenerate bases. In a still further exemplary embodiment, the second anchor comprises one or more degenerate bases at one or both termini and/or within an interior region of its sequence.

In a further embodiment, a set of probes will also comprise one or more groups of sequencing probes for base determination in one or more detection positions with a target nucleic acid. In one embodiment, the set comprises enough different groups of sequencing probes to identify about 1 to about 20 positions within a target nucleic acid. In a further exemplary embodiment, the set comprises enough groups of sequencing probes to identify about 2 to about 18, about 3 to about 16, about 4 to about 14, about 5 to about 12, about 6 to about 10, and about 7 to about 8 positions within a target nucleic acid.

In further exemplary embodiments, 10 pools of labeled or tagged probes will be used in accordance with the invention. In still further embodiments, sets of probes will include two or more anchors with different sequences. In yet further embodiments, sets of probes will include 3, 4, 5, 6, 7, 8, 9, 10, 11, 12, 13, 14, 15 or more anchors with different sequences.

In a further exemplary embodiment, a set of probes is provided comprising one or more groups of sequencing probes and three anchors. The first anchor is complementary to a first region of an adaptor, the second anchor is complementary to a second region of an adaptor, and the second region and the first region are adjacent to each other. The third anchor comprises three or more degenerate nucleotides and is able to hybridize to nucleotides in the target nucleic acid adjacent to the adaptor. The third anchor may also in some embodiments be complementary to a third region of the adaptor, and that third region may be adjacent to the second region, such that the second anchor is flanked by the first and third anchors.

In some embodiments, sets of anchor and/or sequencing probes will comprise variable concentrations of each type of probe, and the variable concentrations may in part depend on the degenerate bases that may be contained in the anchors. For example, probes that will have lower hybridization stability, such as probes with greater numbers of A's and/or T's, can be present in higher relative concentrations as a way to offset their lower stabilities. In further embodiments, these differences in relative concentrations are established by preparing smaller pools of probes independently and then mixing those independently generated pools of probes in the proper amounts.

Improving Specificity and Fidelity of Ligation Reactions

In some aspects, the ligation reactions used in cPAL methods of the invention are modified to include elements for increasing the fidelity of ligation of two nucleic acids adjacently hybridized to a target nucleic acid. In some embodiments, such methods include adding a substance that preferentially increases the stability of double stranded nucleic acids, generally by binding preferentially to double stranded nucleic acids (“double stranded binding moieties”). In some embodiments, an intercalator is used and is added to the ligation reaction mix. “Intercalating agent” or “intercalator” as used herein refers to a substance capable of insertion between adjacent base pairs in a nucleic acid duplex, e.g. that preferentially binds to double-stranded nucleic acids over single stranded nucleic acids Similarly, as will be appreciated by those in the art, minor- and major-groove binding moieties can also be used.

In specific aspects, the intercalator includes but is not limited to ethidium bromide, dihydroethidium, ethidium homodimer-1, ethidium homodimer-2, acridine, propidium iodide, YOYO-1 or TOTO-1, proflavine, daunomycin, doxorubicin, POPO-1, POPO-3, BOBO-1, BOBO-3, Psoralen, Actinomycin D, SYBR Green or thalidomide, and can be fluorescent or non-fluorescent. In a very specific aspect, the intercalator is ethidium bromide. Preferred ranges of ethidium bromide for use in the present invention include from 0.1 ng/μl to about 20.0 ng/μl, and more preferably from about 2.5 ng/μl to about 15.0 ng/μl, even more preferably from about 5.0 ng/μl to about 10.0 ng/μl.

In a further embodiment, the invention provides a method for determining an identity of a base at a position in a target nucleic acid comprising: providing library constructs comprising target nucleic acid and at least one adaptor, wherein the target nucleic acid has a position to be interrogated; hybridizing anchors to the adaptors in the library constructs; hybridizing a pool of sequencing probes to the target nucleic acid; ligating the sequencing probes to the anchors in the presence of a double stranded binding moiety such as an intercalator, wherein the sequencing probe that is complementary to the target nucleic acid will ligate efficiently to an anchor; and determining which sequencing probe is ligated to the anchor so as to determine a sequence of the target nucleic acid. In specific aspects, the unligated sequencing probes are discarded before sequence determination. In a preferred aspect, these steps are repeated until a desired number of bases have been determined.

In a still further embodiment, the invention provides a method for synthesizing nucleic acid library constructs comprising: obtaining target nucleic acids; ligating a first adaptor to the target nucleic acids to produce first library constructs, wherein the first adaptor comprises a restriction endonuclease recognition site for an enzyme that binds in the adaptor but cleaves in the target nucleic acid; amplifying the first library constructs; circularizing the first library constructs; digesting the library constructs with a restriction endonuclease that recognizes the restriction endonuclease recognition site the first adaptor; and ligating a second adaptor to the library constructs to produce second library constructs, wherein one or more of these steps comprise an intercalator in a reaction mix. In a specific aspect, these steps can be repeated until a desired number of interspersed adaptors have been ligated to the target nucleic acids.

In a further embodiment, the invention provides a method for enhancing the selectivity of combined polymerase reactions and ligation reactions, comprising: hybridizing a nucleic acid to a primer; subjecting said hybridized nucleic acid to an extension reaction by extending the primer with a polymerizing enzyme to form a primer extension product, and ligating one end of the extended primer product to a double-stranded nucleic acid, wherein the extension reaction and the ligation reaction are performed in the presence of an intercalating agent. In specific aspects, the double-stranded nucleic acid to which the primer extension product is ligated is the opposite end of the extended primer product. In other aspects, the primer extension product is ligated to a separate nucleic acid. In one specific aspect, the separate nucleic acid is an adaptor. Such methods are useful in the production of nucleic acid libraries as described above.

As discussed in further detail herein, in some embodiments, arrayed targets are hybridized with anchors followed by washing and discarding of excess anchor. The arrays are then hybridized with a mix of T4 DNA ligase and 9-mer fluorescent sequencing probes labeled at either the 3′ or 5′ end. The 9-mer sequencing probes engage in ligation with the anchor oligonucleotides in the presence of T4 ligase, resulting in the formation of a stable hybrid and the association of fluorophore with the anchor and target nucleic acid in a sequence-specific manner. Optionally included in such ligation reactions are double stranded binding moieties such as ethidium bromide, which can be present at varying concentrations, including from about 1 ng/ul to 10 ng/ul. Alternative intercalating agents include but are not limited to dihydroethidium, ethidium homodimer-1, ethidium homodimer-2, acridine, propidium iodide, YOYO-1 or TOTO-1, proflavine, daunomycin, doxorubicin, and thalidomide.

Signal intensity if affected by the concentration of the intercalator present in the reaction. For example, increasing ethidium bromide concentration in a ligation reaction from 1 ng/ul to 10 ng/ul results in a decrease of overall signal intensity of all 4 fluorescent probes. The decrease in signal intensity may reflect the destabilizing action of ethidium bromide on duplex DNA and suggest a mechanism for increased color purity. When a destabilizing force is applied to the duplex the addition of a mismatch has the effect of producing a greater destabilization than if the mismatch was added to a non-destabilized duplex. Decreased signal intensity is not itself detrimental, and may be compensated for by appropriate sensitivity of the measuring instrument.

Other Sequencing Methods

In one aspect, methods and compositions of the present invention are used in combination with techniques such as those described in WO2007120208, WO2006073504, WO2007133831, and US2007099208, and U.S. Patent Application Ser. Nos. 60/992,485; 61/026,337; 61/035,914; 61/061,134; 61/116,193; 61/102,586; 12/265,593; 12/266,385; 11/938,096; 11/981,804; 11/981,797; 11/981,793; 11/981,767; 11/981,761; 11/981,730; 11/981,685; 11/981,661; 11/981,607; 11/981,605; 11/927,388; 11/927,356; 11/679,124; 11/541,225; 10/547,214; 11/451,692; and 11/451,691, all of which are incorporated herein by reference in their entirety for all purposes and in particular for all teachings related to sequencing, particularly sequencing of concatemers.

In a further aspect, sequences of DNBs are identified using sequencing methods known in the art, including, but not limited to, hybridization-based methods, such as disclosed in Drmanac, U.S. Pat. Nos. 6,864,052; 6,309,824; and 6,401,267; and Drmanac et al, U.S. patent publication 2005/0191656, and sequencing-by-synthesis methods, e.g., Nyren et al, U.S. Pat. No. 6,210,891; Ronaghi, U.S. Pat. No. 6,828,100; Ronaghi et al (1998), Science, 281: 363-365; Balasubramanian, U.S. Pat. No. 6,833,246; Quake, U.S. Pat. No. 6,911,345; Li et al, Proc. Natl. Acad. Sci., 100: 414-419 (2003); Smith et al, PCT publication WO 2006/074351; Bowers et al., Nat. Methods 6:593-595 (2009); and Thompson et al., Curr. Protoc. Mol. Biol., Chapter 7:Unit 7.10 (2010); and ligation-based methods, e.g. Shendure et al (2005), Science, 309: 1728-1739, and Macevicz, U.S. Pat. No. 6,306,597; wherein each of these references is herein incorporated by reference in its entirety for all purposes and in particular teachings regarding the figures, legends and accompanying text describing the compositions, methods of using the compositions and methods of making the compositions, particularly with respect to sequencing.

In some embodiments, nucleic acid templates of the invention, as well as DNBs generated from those templates, are used in sequencing-by-synthesis methods. The efficiency of sequencing by synthesis methods utilizing nucleic acid templates of the invention is increased over conventional sequencing by synthesis methods utilizing nucleic acids that do not comprise multiple interspersed adaptors. Rather than a single long read, nucleic acid templates of the invention allow for multiple short reads that each start at one of the adaptors in the template. Such short reads consume fewer labeled dNTPs, thus saving on the cost of reagents. In addition, sequencing by synthesis reactions can be performed on DNB arrays, which provide a high density of sequencing targets as well as multiple copies of monomeric units. Such arrays provide detectable signals at the single molecule level while at the same time providing an increased amount of sequence information, because most or all of the DNB monomeric units will be extended without losing sequencing phase. The high density of the arrays also reduces reagent costs—in some embodiments the reduction in reagent costs can be from about 30 to about 40% over conventional sequencing by synthesis methods. In some embodiments, the interspersed adaptors of the nucleic acid templates of the invention provide a way to combine about two to about ten standard reads if inserted at distances of from about 30 to about 100 bases apart from one another. In such embodiments, the newly synthesized strands will not need to be stripped off for further sequencing cycles, thus allowing the use of a single DNB array through about 100 to about 400 sequencing by synthesis cycles.

In some embodiments of the present invention, the unchained cPAL sequencing methods are extended to include two or more ligation events with sequencing probes. For example, after a first ligation product comprising a first sequencing probe ligated to a construct comprising one or more anchors is detected, a second sequencing probe may be hybridized to the nucleic acid target at a position adjacent to that first ligation product and ligated to the first sequencing probe. The second sequencing probe may then be detected. As will be appreciated, multiple sequencing probes may undergo such a hybridization-ligation cycle. The resultant ligation products can then be removed from the target and another round of cPAL sequencing as described herein can be conducted. In such embodiments, the unchained cPAL sequencing method is partially combined with a chained method utilizing one or more additional sequencing probes. As will be appreciated, each new sequencing probe can be detected using methods known in the art. For example, if the sequencing probes are labeled with fluorophores, after each ligated sequencing probe is detected, the attached fluorophore can be cleaved, allowing for the second sequencing probe added to the “chain” to be detected without interference from the label on the first sequencing probe.

Two-Phase Sequencing

In one aspect, the present invention provides methods for “two-phase” sequencing, which is also referred to herein as “shotgun sequencing”. Such methods are described in U.S. patent application Ser. No. 12/325,922, filed Dec. 1, 2008, which is hereby incorporated by reference in its entirety for all purposes and in particular for all teachings related to two-phase or shotgun sequencing.

Generally, two phase-sequencing methods of use in the present invention comprise the following steps: (a) sequencing the target nucleic acid to produce a primary target nucleic acid sequence that comprises one or more sequences of interest; (b) synthesizing a plurality of target-specific oligonucleotides, wherein each of said plurality of target-specific oligonucleotides corresponds to at least one of the sequences of interest; (c) providing a library of fragments of the target nucleic acid (or constructs that comprise such fragments and that may further comprise, for example, adaptors and other sequences as described herein) that hybridize to the plurality of target-specific oligonucleotides; and (d) sequencing the library of fragments (or constructs that comprise such fragments) to produce a secondary target nucleic acid sequence. In order to close gaps due to missing sequence or resolve low confidence base calls in a primary sequence of genomic DNA, such as human genomic DNA, the number of target-specific oligonucleotides that are synthesized for these methods may be from about ten thousand to about one million; thus the present invention contemplates the use of at least about 10,000 target-specific oligonucleotides, or about 25,000, or about 50,000, or about 100,000, or about 20,000, or about 50,000, or about 100,000, or about 200,000 or more.

In saying that the plurality of target-specific oligonucleotides “corresponds to” at least one of the sequences of interest, it is meant that such target-specific oligonucleotides are designed to hybridize to the target nucleic acid in proximity to, including but not limited to, adjacent to, the sequence of interest such that there is a high likelihood that a fragment of the target nucleic acid that hybridizes to such an oligonucleotides will include the sequence of interest. Such target-specific oligonucleotides are therefore useful for hybrid capture methods to produce a library of fragments enriched for such sequences of interest, as sequencing primers for sequencing the sequence of interest, as amplification primers for amplifying the sequence of interest, or for other purposes.

In shotgun sequencing and other sequencing methods according to the present invention, after assembly of sequencing reads, to the skilled person it is apparent from the assembled sequence that gaps exist or that there is low confidence in one or more bases or stretches of bases at a particular site in the sequence. Sequences of interest, which may include such gaps, low confidence sequence, or simply different sequences at a particular location (i.e., a change of one or more nucleotides in target sequence), can also be identified by comparing the primary target nucleic acid sequence to a reference sequence.

According to one embodiment of such methods sequencing the target nucleic acid to produce a primary target nucleic acid sequence comprises computerized input of sequence readings and computerized assembly of the sequence readings to produce the primary target nucleic acid sequence. In addition, design of the target-specific oligonucleotides can be computerized, and such computerized synthesis of the target-specific oligonucleotides can be integrated with the computerized input and assembly of the sequence readings and design of the target-specific oligonucleotides. This is especially helpful since the number of target-specific oligonucleotides to be synthesized can be in the tens of thousands or hundreds of thousands for genomes of higher organisms such as humans, for example. Thus the invention provides automated integration of the process of creating the oligonucleotide pool from the determined sequences and the regions identified for further processing. In some embodiments, a computer-driven program uses the identified regions and determined sequence near or adjacent to such identified regions to design oligonucleotides to isolate and/or create new fragments that cover these regions. The oligonucleotides can then be used as described herein to isolate fragments, either from the first sequencing library, from a precursor of the first sequencing library, from a different sequencing library created from the same target nucleic acid, directly from target nucleic acids, and the like. In further embodiments, this automated integration of identifying regions for further analysis and isolating/creating the second library defines the sequence of the oligonucleotides within the oligonucleotide pool and directs synthesis of these oligonucleotides.

In some embodiments of the two phase sequencing methods of the invention, a releasing process is performed after the hybrid capture process, and in other aspects of the technology, an amplification process is performed before the second sequencing process.

In still further embodiments, some or all regions are identified in the identifying step by comparison of determined sequences with a reference sequence. In some aspects, the second shotgun sequencing library is isolated using a pool of oligonucleotides comprising oligonucleotides based on a reference sequence. Also, in some aspects, the pool of oligonucleotides comprises at least 1000 oligonucleotides of different sequence, in other aspects, the pool of oligonucleotides comprises at least 10,000, 25,000, 50,000, 75,000, or 100,000 or more oligonucleotides of different sequence

In some aspects of the invention, one or more of the sequencing processes used in this two-phase sequencing method is performed by sequencing-by-ligation, and in other aspects, one or more of the sequencing processes is performed by sequencing-by-hybridization or sequencing-by-synthesis.

In certain aspects of the invention, between about 1 to about 30% of the complex target nucleic acid is identified as having to be re-sequenced in Phase II of the methods, and in other aspects, between about 1 to about 10% of the complex target nucleic acid is identified as having to be re-sequenced in Phase II of the methods. In some aspects, coverage for the identified percentage of complex target nucleic acid is between about 25× to about 100×.

In further aspects, 1 to about 10 target-specific selection oligonucleotides are defined and synthesized for each target nucleic acid region that is re-sequenced in Phase II of the methods; in other aspects, about 3 to about 6 target-specific selection oligonucleotides are defined for each target nucleic acid region that is re-sequenced in Phase II of the methods.

In still further aspects of the technology, the target-specific selection oligonucleotides are identified and synthesized by an automated process, wherein the process that identifies regions of the complex nucleic acid missing nucleic acid sequence or having low confidence nucleic acid sequence and defines sequences for the target-specific selection oligonucleotides communicates with oligonucleotide synthesis software and hardware to synthesize the target-specific selection oligonucleotides. In other aspects of the technology, the target-specific selection oligonucleotides are between about 20 and about 30 bases in length, and in some aspects are unmodified.

Not all regions identified for further analysis may actually exist in the complex target nucleic acid. One reason for predicted lack of coverage in a region may be that a region expected to be in the complex target nucleic acid may actually not be present (e.g., a region may be deleted or re-arranged in the target nucleic acid), and thus not all oligonucleotides produced from the pool may isolate a fragment for inclusion in the second shotgun sequencing library. In some embodiments, at least one oligonucleotide will be designed and created for each region identified for further analysis. In further embodiments, an average of three or more oligonucleotides will be provided for each region identified for further analysis. It is a feature of the invention that the pool of oligonucleotides can be used directly to create the second shotgun sequencing library by polymerase extension of the oligonucleotides using templates derived from a target nucleic acid. It is another feature of the invention that the pool of oligonucleotides can be used directly to create amplicons via circle dependent replication using the oligonucleotide pools and circle dependent replication. It is another feature of the invention that the methods will provide sequencing information to identify absent regions of interest, e.g. predicted regions that were identified for analysis but which do not exist, e.g., due to a deletion or rearrangement.

The above described embodiments of the two-phase sequencing method can be used in combination with any of the nucleic acid constructs and sequencing methods described herein and known in the art.

SNP Detection

Methods and compositions discussed above can in further embodiments be used to detect specific sequences in nucleic acid constructs such as DNBs. In particular, cPAL methods utilizing sequencing and anchors can be used to detect polymorphisms or sequences associated with a genetic mutation, including single nucleotide polymorphisms (SNPs). For example, to detect the presence of a SNP, two sets of differentially labeled sequencing probes can be used, such that detection of one probe over the other indicates whether a polymorphism present in the sample. Such sequencing probes can be used in conjunction with anchors in methods similar to the cPAL methods described above to further improve the specificity and efficiency of detection of the SNP.

### Long Fragment Read Technology

Overview

Individual human genomes are diploid in nature, with half of the homologous chromosomes being derived from each parent. The context in which variations occur on each individual chromosome can have profound effects on the expression and regulation of genes and other transcribed regions of the genome. Further, determining if two potentially detrimental mutations occur within one or both alleles of a gene is of paramount clinical importance.

Current methods for whole-genome sequencing lack the ability to separately assemble parental chromosomes in a cost-effective way and describe the context (haplotypes) in which variations co-occur. Simulation experiments show that chromosome-level haplotyping requires allele linkage information across a range of at least 70-100 kb. This cannot be achieved with existing technologies that use amplified DNA, which are be limited to reads less than 1000 bases due to difficulties in uniform amplification of long DNA molecules and loss of linkage information in sequencing. Mate-pair technologies can provide an equivalent to the extended read length but are limited to less than 10 kb due to inefficiencies in making such DNA libraries (due to the difficulty of circularizing DNA longer than a few kb in length). This approach also needs extreme read coverage to link all heterozygotes.

Single molecule sequencing of greater than 100 kb DNA fragments would be useful for haplotyping if processing such long molecules were feasible, if the accuracy of single molecule sequencing were high, and detection/instrument costs were low. This is very difficult to achieve on short molecules with high yield, let alone on 100 kb fragments.

Most recent human genome sequencing has been performed on short read-length (<200 bp), highly parallelized systems starting with hundreds of nanograms of DNA. These technologies are excellent at generating large volumes of data quickly and economically. Unfortunately, short reads, often paired with small mate-gap sizes (500 bp-10 kb), eliminate most SNP phase information beyond a few kilobases (McKernan et al., Genome Res. 19:1527, 2009). Furthermore, it is very difficult to maintain long DNA fragments in multiple processing steps without fragmenting as a result of shearing.

At the present time three personal genomes, those of J. Craig Venter (Levy et al., PLoS Biol. 5:e254, 2007), a Gujarati Indian (HapMap sample NA20847; Kitzman et al., Nat. Biotechnol. 29:59, 2011), and two Europeans (Max Planck One [MP1]; Suk et al., Genome Res., 2011; genome.cshlp.org/content/early/2011/09/02/gr.125047.111.full.pdf; and HapMap Sample NA 12878; Duitama et al., Nucl. Acids Res. 40:2041-2053, 2012) have been sequenced and assembled as diploid. All have involved cloning long DNA fragments into constructs in a process similar to the bacterial artificial chromosome (BAC) sequencing used during construction of the human reference genome (Venter et al., Science 291:1304, 2001; Lander et al., Nature 409:860, 2001). While these processes generate long phased contigs (N50s of 350 kb [Levy et al., PLoS Biol. 5:e254, 2007], 386 kb [Kitzman et al., Nat. Biotechnol. 29:59-63, 2011] and 1 Mb [Suk et al., Genome Res. 21:1672-1685, 2011]) they require a large amount of initial DNA, extensive library processing, and are too expensive to use in a routine clinical environment.

Additionally, whole chromosome haplotyping has been demonstrated through direct isolation of metaphase chromosomes (Zhang et al., Nat. Genet. 38:382-387, 2006; Ma et al., Nat. Methods 7:299-301, 2010; Fan et al., Nat. Biotechnol. 29:51-57, 2011; Yang et al., Proc. Natl. Acad. Sci. USA 108:12-17, 2011). These methods are excellent for long-range haplotyping but have yet to be used for whole-genome sequencing and require preparation and isolation of whole metaphase chromosomes, which can be challenging for some clinical samples.

LFR methods overcome these limitations. LFR includes DNA preparation and tagging, along with related algorithms and software, to enable an accurate assembly of separate sequences of parental chromosomes (i.e., complete haplotyping) in diploid genomes at significantly reduced experimental and computational costs.

LFR is based on the physical separation of long fragments of genomic DNA (or other nucleic acids) across many different aliquots such that there is a low probability of any given region of the genome of both the maternal and paternal component being represented in the same aliquot. By placing a unique identifier in each aliquot and analyzing many aliquots in the aggregate, DNA sequence data can be assembled into a diploid genome, e.g., the sequence of each parental chromosome can be determined. LFR does not require cloning fragments of a complex nucleic acid into a vector, as in haplotyping approaches using large-fragment (e.g., BAC) libraries. Nor does LFR require direct isolation of individual chromosomes of an organism. Finally, LFR can be performed on an individual organism and does not require a population of the organism in order to accomplish haplotype phasing.

As used herein, the term “vector” means a plasmid or viral vector into which a fragment of foreign DNA is inserted. A vector is used to introduce foreign DNA into a suitable host cell, where the vector and inserted foreign DNA replicates due to the presence in the vector of, for example, a functional origin of replication or autonomously replicating sequence. As used herein, the term “cloning” refers to the insertion of a fragment of DNA into a vector and replication of the vector with inserted foreign DNA in a suitable host cell.

LFR can be used together with the sequencing methods discussed in detail herein and, more generally, as a preprocessing method with any sequencing technology known in the art, including both short-read and longer-read methods. LFR also can be used in conjunction with various types of analysis, including, for example, analysis of the transcriptome, methylome, etc. Because it requires very little input DNA, LFR can be used for sequencing and haplotyping one or a small number of cells, which can be particularly important for cancer, prenatal diagnostics, and personalized medicine. This can facilitate the identification of familial genetic disease, etc. By making it possible to distinguish calls from the two sets of chromosomes in a diploid sample, LFR also allows higher confidence calling of variant and non-variant positions at low coverage. Additional applications of LFR include resolution of extensive rearrangements in cancer genomes and full-length sequencing of alternatively spliced transcripts.

LFR can be used to process and analyze complex nucleic acids, including but not limited to genomic DNA, that is purified or unpurified, including cells and tissues that are gently disrupted to release such complex nucleic acids without shearing and overly fragmenting such complex nucleic acids.

In one aspect, LFR produces virtual read lengths of approximately 100-1000 kb in length.

In addition, LFR can also dramatically reduce the computational demands and associated costs of any short read technology. Importantly, LFR removes the need for extending sequencing read length if that reduces the overall yield. An additional benefit of LFR is a substantial (10- to 1000-fold) reduction in errors or questionable base calls that can result from current sequencing technologies, usually one per 100 kb, or 30,000 false positive calls per human genome, and a similar number of undetected variants per human genome. This dramatic reduction in errors minimizes the need for follow up confirmation of detected variants and facilitates adoption of human genome sequencing for diagnostic applications.

In addition to being applicable to all sequencing platforms, LFR-based sequencing can be applied to any application, including without limitation, the study of structural rearrangements in cancer genomes, full methylome analysis including the haplotypes of methylated sites, and de novo assembly applications for metagenomics or novel genome sequencing, even of complex polyploid genomes like those found in plants.

LFR provides the ability to obtain actual sequences of individual chromosomes as opposed to just the consensus sequences of parental or related chromosomes (in spite of their high similarities and presence of long repeats and segmental duplications). To generate this type of data, the continuity of sequence is in general established over long DNA ranges such as 100 kb to 1 Mb.

A further aspect of the invention includes software and algorithms for efficiently utilizing LFR data for whole chromosome haplotype and structural variation mapping and false positive/negative error correcting to fewer than 300 errors per human genome.

In a further aspect, LFR techniques of the invention reduce the complexity of DNA in each aliquot by 100-1000 fold depending on the number of aliquots and cells used. Complexity reduction and haplotype separation in >100 kb long DNA can be helpful in more efficiently and cost effectively (up to 100-fold reduction in cost) assembling and detect all variations in human and other diploid genomes.

LFR methods described herein can be used as a pre-processing step for sequencing diploid genomes using any sequencing methods known in the art. The LFR methods described herein may in further embodiments be used on any number of sequencing platforms, including for example without limitation, polymerase-based sequencing-by-synthesis (e.g., HiSeq 2500 system, Illumina, San Diego, Calif.), ligation-based sequencing (e.g., SOLiD 5500, Life Technologies Corporation, Carlsbad, Calif.), ion semiconductor sequencing (e.g., Ion PGM or Ion Proton sequencers, Life Technologies Corporation, Carlsbad, Calif.), zero-mode waveguides (e.g., PacBio RS sequencer, Pacific Biosciences, Menlo Park, Calif.), nanopore sequencing (e.g., Oxford Nanopore Technologies Ltd., Oxford, United Kingdom), pyrosequencing (e.g., 454 Life Sciences, Branford, Conn.), or other sequencing technologies. Some of these sequencing technologies are short-read technologies, but others produce longer reads, e.g., the GS FLX+ (454 Life Sciences; up to 1000 bp), PacBio RS (Pacific Biosciences; approximately 1000 bp) and nanopore sequencing (Oxford Nanopore Technologies Ltd.; 100 kb). For haplotype phasing, longer reads are advantageous, requiring much less computation, although they tend to have a higher error rate and errors in such long reads may need to be identified and corrected according to methods set forth herein before haplotype phasing.

According to one embodiment of the invention, the basic steps of LFR include: (1) separating long fragments of a complex nucleic acid (e.g., genomic DNA) into aliquots, each aliquot containing a fraction of a genome equivalent of DNA; (2) amplifying the genomic fragments in each aliquot; (3) fragmenting the amplified genomic fragments to create short fragments (e.g., ˜500 bases in length in one embodiment) of a size suitable for library construction; (4) tagging the short fragments to permit the identification of the aliquot from which the short fragments originated; (5) pooling the tagged fragments; (6) sequencing the pooled, tagged fragments; and (7) analyzing the resulting sequence data to map and assemble the data and to obtain haplotype information. According to one embodiment, LFR uses a 384-well plate with 10-20% of a haploid genome in each well, yielding a theoretical 19-38× physical coverage of both the maternal and paternal alleles of each fragment. An initial DNA redundancy of 19-38× ensures complete genome coverage and higher variant calling and phasing accuracy. LFR avoids subcloning of fragments of a complex nucleic acid into a vector or the need to isolate individual chromosomes (e.g., metaphase chromosomes), and it can be fully automated, making it suitable for high-throughput, cost-effective applications.

We have also developed techniques for using LFR for error reduction and other purposes as detailed herein. LFR methods have been described in U.S. patent application Ser. Nos. 12/329,365 and 13/447,087, US Pat. Publications US 2011-0033854 and 2009-0176234, and U.S. Pat. Nos. 7,901,890, 7,897,344, 7,906,285, 7,901,891, and 7,709,197, all of which are hereby incorporated by reference in their entirety.

As used herein, the term “haplotype” means a combination of alleles at adjacent locations (loci) on the chromosome that are transmitted together or, alternatively, a set of sequence variants on a single chromosome of a chromosome pair that are statistically associated. Every human individual has two sets of chromosomes, one paternal and the other maternal. Usually DNA sequencing results only in genotypic information, the sequence of unordered alleles along a segment of DNA. Inferring the haplotypes for a genotype separates the alleles in each unordered pair into two separate sequences, each called a haplotype. Haplotype information is necessary for many different types of genetic analysis, including disease association studies and making inference on population ancestries.

As used herein, the term “phasing” (or resolution) means sorting sequence data into the two sets of parental chromosomes or haplotypes. Haplotype phasing refers to the problem of receiving as input a set of genotypes for one individual or a population (i.e., more than one individual) and outputting a pair of haplotypes for each individual, one being paternal and the other maternal. Phasing can involve resolving sequence data over a region of a genome, or as little as two sequence variants in a read or contig, which may be referred to as local phasing, or microphasing. It can also involve phasing of longer contigs, generally including greater than about ten sequence variants, or even a whole genome sequence, which may be referred to as “universal phasing.” Optionally, phasing sequence variants takes place during genome assembly.

Aliquoting Fractions of a Genome Equivalent of the Complex Nucleic Acid

The LFR process is based upon the stochastic physical separation of a genome in long fragments into many aliquots such that each aliquot contains a fraction of a haploid genome. As the fraction of the genome in each pool decreases, the statistical likelihood of having a corresponding fragment from both parental chromosomes in the same pool dramatically diminishes.

In some embodiments, a 10% genome equivalent is aliquoted into each well of a multiwell plate. In other embodiments, 1% to 50% of a genome equivalent of the complex nucleic acid is aliquoted into each well. As noted above, the number of aliquots and genome equivalents can depend on the number of aliquots, original fragment size, or other factors. Optionally, a double-stranded nucleic acid (e.g., a human genome) is denatured before aliquoting; thus single-stranded complements may be apportioned to different aliquots.

For example, at 0.1 genome equivalents per aliquot (approximately 0.66 picogram, or pg, of DNA, at approximately 6.6 pg per human genome) there is a 10% chance that two fragments will overlap and a 50% chance those fragments will be derived from separate parental chromosomes; this yields a 95% of the base pairs in an aliquot are non-overlapping, i.e., 5% overall chance that a particular aliquot will be uninformative for a given fragment, because the aliquot contains fragments deriving from both maternal and paternal chromosomes. Aliquots that are uninformative can be identified because the sequence data resulting from such aliquots contains an increased amount of “noise,” that is, the impurity in the connectivity matrix between pairs of hets. Fuzzy interference system (FIS) allows robustness against a certain degree of impurity, i.e., it can make correct connection despite the impurity (up to a certain degree). Even smaller amounts of genomic DNA can be used, particularly in the context of micro- or nanodroplets or emulsions, where each droplet could include one DNA fragment (e.g., a single 50 kb fragment of genomic DNA or approximately 1.5×10⁻⁵ genome equivalents). Even at 50 percent of a genome equivalent, a majority of aliquots would be informative. At higher levels, e.g., 70 percent of a genome equivalent, wells that are informative can be identified and used. According to one aspect of the invention, 0.000015, 0.0001, 0.001, 0.01, 0.1, 1, 5, 10, 15, 20, 25, 40, 50, 60, or 70 percent of a genome equivalent of the complex nucleic acid is present in each aliquot.

It should be appreciated that the dilution factor can depend on the original size of the fragments. That is, using gentle techniques to isolate genomic DNA, fragments of roughly 100 kb can be obtained, which are then aliquoted. Techniques that allow larger fragments result in a need for fewer aliquots, and those that result in shorter fragments may require more dilution.

We have successfully performed all six enzymatic steps in the same reaction without DNA purification, which facilitates miniaturization and automation and makes it feasible to adapt LFR to a wide variety of platforms and sample preparation methods.

According to one embodiment, each aliquot is contained in a separate well of a multi-well plate (for example, a 384 well plate). However, any appropriate type of container or system known in the art can be used to hold the aliquots, or the LFR process can be performed using microdroplets or emulsions, as described herein. According to one embodiment of the invention, volumes are reduced to sub-microliter levels. In one embodiment, automated pipetting approaches can be used in 1536 well formats.

In general, as the number of aliquots increases, for instance to 1536, and the percent of the genome decreases down to approximately 1% of a haploid genome, the statistical support for haplotypes increases dramatically, because the sporadic presence of both maternal and paternal haplotypes in the same well diminishes. Consequently, a large number of small aliquots with a negligent frequency of mixed haplotypes per aliquot allows for the use of fewer cells. Similarly, longer fragments (e.g., 300 kb or longer) help bridge over segments lacking heterozygous loci.

Nanoliter (nl) dispensing tools (e.g., Hamilton Robotics Nano Pipetting head, TTP LabTech Mosquito, and others) that provide noncontact pipeting of 50-100 nl can be used for fast and low cost pipetting to make tens of genome libraries in parallel. The increase in the number of aliquots (as compared with a 384 well plate) results in a large reduction in the complexity of the genome within each well, reducing the overall cost of computing over 10-fold and increasing data quality. Additionally, the automation of this process increases the throughput and lowers the hands-on cost of producing libraries.

LFR Using Smaller Aliquot Volumes, Including Microdroplets and Emulsions

Even further cost reductions and other advantages can be achieved using microdroplets. In some embodiments, LFR is performed with combinatorial tagging in emulsion or microfluidic devices. A reduction of volumes down to picoliter levels in 10,000 aliquots can achieve an even greater cost reduction due to lower reagent and computational costs.

In one embodiment, LFR uses 10 microliter (μl) volume of reagents per well in a 384 well format. Such volumes can be reduced to by using commercially available automated pipetting approaches in 1536 well formats, for example. Further volume reductions can be achieved using nanoliter (nl) dispensing tools (e.g., Hamilton Robotics Nano Pipetting head, TTP LabTech Mosquito, and others) that provide noncontact pipeting of 50-100 nl can be used for fast and low cost pipetting to make tens of genome libraries in parallel. Increasing the number of aliquots results in a large reduction in the complexity of the genome within each well, reducing the overall cost of computing and increasing data quality. Additionally, the automation of this process increases the throughput and lower the cost of producing libraries.

In further embodiments, unique identification of each aliquot is achieved with 8-12 base pair error correcting barcodes. In some embodiments, the same number of adaptors as wells is used.

In further embodiments, a novel combinatorial tagging approach is used based on two sets of 40 half-barcode adapters. In one embodiment, library construction involves using two different adaptors. A and B adapters are easily be modified to each contain a different half-barcode sequence to yield thousands of combinations. In a further embodiment, the barcode sequences are incorporated on the same adapter. This can be achieved by breaking the B adaptor into two parts, each with a half barcode sequence separated by a common overlapping sequence used for ligation. The two tag components have 4-6 bases each. An 8-base (2×4 bases) tag set is capable of uniquely tagging 65,000 aliquots. One extra base (2×5 bases) will allow error detection and 12 base tags (2×6 bases, 12 million unique barcode sequences) can be designed to allow substantial error detection and correction in 10,000 or more aliquots using Reed-Solomon design. In exemplary embodiments, both 2×5 base and 2×6 base tags, including use of degenerate bases (i.e., “wild-cards”), are employed to achieve optimal decoding efficiency.

A reduction of volumes down to picoliter levels (e.g., in 10,000 aliquots) can achieve an even greater reduction in reagent and computational costs. In some embodiments, this level of cost reduction and extensive aliquoting is accomplished through the combination of the LFR process with combinatorial tagging to emulsion or microfluidic-type devices. The ability to perform all enzymatic steps in the same reaction without DNA purification facilitates the ability to miniaturize and automate this process and results in adaptability to a wide variety of platforms and sample preparation methods.

In one embodiment, LFR methods are used in conjunction with an emulsion-type device. A first step to adapting LFR to an emulsion type device is to prepare an emulsion reagent of combinatorial barcode tagged adapters with a single unique barcode per droplet. Two sets of 100 half-barcodes is sufficient to uniquely identify 10,000 aliquots. However, increasing the number of half-barcode adapters to over 300 can allow for a random addition of barcode droplets to be combined with the sample DNA with a low likelihood of any two aliquots containing the same combination of barcodes. Combinatorial barcode adapter droplets can be made and stored in a single tube as a reagent for thousands of LFR libraries.

In one embodiment, the present invention is scaled from 10,000 to 100,000 or more aliquot libraries. In a further embodiment, the LFR method is adapted for such a scale-up by increasing the number of initial half barcode adapters. These combinatorial adapter droplets are then fused one-to-one with droplets containing ligation ready DNA representing less than 1% of the haploid genome. Using a conservative estimate of 1 nl per droplet and 10,000 drops this represents a total volume of 10 μl for an entire LFR library.

Recent studies have also suggested an improvement in GC bias after amplification (e.g., by MDA) and a reduction in background amplification by decreasing the reaction volumes down to nanoliter size.

There are currently several types of microfluidics devices (e.g., devices sold by Advanced Liquid Logic, Morrisville, N.C.) or pico/nano-droplet (e.g., RainDance Technologies, Lexington, Mass.) that have pico-/nano-drop making, fusing (3000/second) and collecting functions and could be used in such embodiments of LFR. In other embodiments, ˜10-20 nanoliter drops are deposited in plates or on glass slides in 3072-6144 format (still a cost effective total MDA volume of 60 μl without losing the computational cost savings or the ability to sequence genomic DNA from a small number of cells) or higher using improved nano-pipeting or acoustic droplet ejection technology (e.g., LabCyte Inc., Sunnyvale, Calif.) or using microfluidic devices (e.g., those produced by Fluidigm, South San Francisco, Calif.) that are capable of handling up to 9216 individual reaction wells. Increasing the number of aliquots results in a large reduction in the complexity of the genome within each well, reducing the overall cost of computing and increasing data quality. Additionally, the automation of this process increases the throughput and lower the cost of producing libraries.

Amplifying

According to one embodiment, the LFR process begins with a short treatment of genomic DNA with a 5′ exonuclease to create 3′ single-stranded overhangs that serve as MDA initiation sites. The use of the exonuclease eliminates the need for a heat or alkaline denaturation step prior to amplification without introducing bias into the population of fragments. Alkaline denaturation can be combined with the 5′ exonuclease treatment, which results in a further reduction in bias. The DNA is then diluted to sub-genome concentrations and aliquoted. After aliquoting the fragments in each well are amplified, e.g., using an MDA method. In certain embodiments, the MDA reaction is a modified phi29 polymerase-based amplification reaction, although another known amplification method can be used.

In some embodiments, the MDA reaction is designed to introduce uracils into the amplification products. In some embodiments, a standard MDA reaction utilizing random hexamers is used to amplify the fragments in each well. In many embodiments, rather than the random hexamers, random 8-mer primers are used to reduce amplification bias in the population of fragments. In further embodiments, several different enzymes can also be added to the MDA reaction to reduce the bias of the amplification. For example, low concentrations of non-processive 5′ exonucleases and/or single-stranded binding proteins can be used to create binding sites for the 8-mers. Chemical agents such as betaine, DMSO, and trehalose can also be used to reduce bias through similar mechanisms.

Fragmentation

According to one embodiment, after amplification of DNA in each well, the amplification products are subjected to a round of fragmentation. In some embodiments the above-described CoRE method is used to further fragment the fragments in each well following amplification. In order to use the CoRE method, the MDA reaction used to amplify the fragments in each well is designed to incorporate uracils into the MDA products. The fragmenting of the MDA products can also be achieved via sonication or enzymatic treatment.

If a CoRE method is used to fragment the MDA products, each well containing amplified DNA is treated with a mix of uracil DNA glycosylase (UDG), DNA glycosylase-lyase endonuclease VIII, and T4 polynucleotide kinase to excise the uracil bases and create single base gaps with functional 5′ phosphate and 3′ hydroxyl groups. Nick translation through use of a polymerase such as Taq polymerase results in double-stranded blunt end breaks, resulting in ligatable fragments of a size range dependent on the concentration of dUTP added in the MDA reaction. In some embodiments, the CoRE method used involves removing uracils by polymerization and strand displacement by phi29.

Following fragmentation of the MDA products, the ends of the resultant fragments can be repaired. Such repairs can be necessary, because many fragmentation techniques can result in termini with overhanging ends and termini with functional groups that are not useful in later ligation reactions, such as 3′ and 5′ hydroxyl groups and/or 3′ and 5′ phosphate groups. In many aspects of the present invention, it is useful to have fragments that are repaired to have blunt ends, and in some cases, it can be desirable to alter the chemistry of the termini such that the correct orientation of phosphate and hydroxyl groups is not present, thus preventing “polymerization” of the target sequences. The control over the chemistry of the termini can be provided using methods known in the art. For example, in some circumstances, the use of phosphatase eliminates all the phosphate groups, such that all ends contain hydroxyl groups. Each end can then be selectively altered to allow ligation between the desired components. One end of the fragments can then be “activated”, in some embodiments by treatment with alkaline phosphatase.

After fragmentation and, optionally, end repair, the fragments are tagged with an adaptor.

Tagging

Generally, the tag adaptor arm is designed in two segments—one segment is common to all wells and blunt end ligates directly to the fragments using methods described further herein. The second segment is unique to each well and contains a “barcode” sequence such that when the contents of each well are combined, the fragments from each well can be identified.

According to one embodiment the “common” adaptor is added as two adaptor arms—one arm is blunt end ligated to the 5′ end of the fragment and the other arm is blunt end ligated to the 3′ end of the fragment. The second segment of the tagging adaptor is a “barcode” segment that is unique to each well. This barcode is generally a unique sequence of nucleotides, and each fragment in a particular well is given the same barcode. Thus, when the tagged fragments from all the wells are re-combined for sequencing applications, fragments from the same well can be identified through identification of the barcode adaptor. The barcode is ligated to the 5′ end of the common adaptor arm. The common adaptor and the barcode adaptor can be ligated to the fragment sequentially or simultaneously. The ends of the common adaptor and the barcode adaptor can be modified such that each adaptor segment will ligate in the correct orientation and to the proper molecule. Such modifications prevent “polymerization” of the adaptor segments or the fragments by ensuring that the fragments are unable to ligate to each other and that the adaptor segments are only able to ligate in the illustrated orientation.

In further embodiments, a three-segment design is utilized for the adaptors used to tag fragments in each well. This embodiment is similar to the barcode adaptor design described above, except that the barcode adaptor segment is split into two segments. This design allows for a wider range of possible barcodes by allowing combinatorial barcode adaptor segments to be generated by ligating different barcode segments together to form the full barcode segment. This combinatorial design provides a larger repertoire of possible barcode adaptors while reducing the number of full size barcode adaptors that need to be generated.

According to one embodiment, after the fragments in each well are tagged, all of the fragments are combined to form a single population. These fragments can then be used to generate nucleic acid templates of the invention for sequencing. The nucleic acid templates generated from these tagged fragments are identifiable as originating from a particular well by the barcode tag adaptors attached to each fragment. Similarly, upon sequencing of the tag, the genomic sequence to which it is attached is also identifiable as originating from the well.

In some embodiments, LFR methods described herein do not include multiple levels or tiers of fragmentation/aliquoting, as described in U.S. patent application Ser. No. 11/451,692, filed Jun. 13, 2006, which is herein incorporated by reference in its entirety for all purposes. That is, some embodiments utilize only a single round of aliquoting, and also allow the repooling of aliquots for a single array, rather than using separate arrays for each aliquot.

LFR Using One or a Small Number of Cells as the Source of Complex Nucleic Acids

According to one embodiment, an LFR method is used to analyze the genome of an individual cell or a small number of cells. The process for isolating DNA in this case is similar to the methods described above, but may occur in a smaller volume.

As discussed above, isolating long fragments of genomic nucleic acid from a cell can be accomplished by a number of different methods. In one embodiment, cells are lysed and the intact nucleic are pelleted with a gentle centrifugation step. The genomic DNA is then released through proteinase K and RNase digestion for several hours. The material can then in some embodiments be treated to lower the concentration of remaining cellular waste—such treatments are well known in the art and can include without limitation dialysis for a period of time (e.g., from 2-16 hours) and/or dilution. Since such methods of isolating the nucleic acid does not involve many disruptive processes (such as ethanol precipitation, centrifugation, and vortexing), the genomic nucleic acid remains largely intact, yielding a majority of fragments that have lengths in excess of 150 kilobases. In some embodiments, the fragments are from about 100 to about 750 kilobases in lengths. In further embodiments, the fragments are from about 150 to about 600, about 200 to about 500, about 250 to about 400, and about 300 to about 350 kilobases in length.

Once the DNA is isolated and before it is aliquoted into individual wells, the genomic DNA must be carefully fragmented to avoid loss of material, particularly to avoid loss of sequence from the ends of each fragment, since loss of such material will result in gaps in the final genome assembly. In some cases, sequence loss is avoided through use of an infrequent nicking enzyme, which creates starting sites for a polymerase, such as phi29 polymerase, at distances of approximately 100 kb from each other. As the polymerase creates the new DNA strand, it displaces the old strand, with the end result being that there are overlapping sequences near the sites of polymerase initiation, resulting in very few deletions of sequence.

In some embodiments, a controlled use of a 5′ exonuclease (either before or during the MDA reaction) can promote multiple replications of the original DNA from the single cell and thus minimize propagation of early errors through copying of copies.

In one aspect, methods of the present invention produce quality genomic data from single cells. Assuming no loss of DNA, there is a benefit to starting with a low number of cells (10 or less) instead of using an equivalent amount of DNA from a large prep. Starting with less than 10 cells and faithfully aliquoting substantially all DNA ensures uniform coverage in long fragments of any given region of the genome. Starting with five or fewer cells allows four times or greater coverage per each 100 kb DNA fragment in each aliquot without increasing the total number of reads above 120 Gb (20 times coverage of a 6 Gb diploid genome). However, a large number of aliquots (10,000 or more) and longer DNA fragments (>200 kb) are even more important for sequencing from a few cells, because for any given sequence there are only as many overlapping fragments as the number of starting cells and the occurrence of overlapping fragments from both parental chromosomes in an aliquot can be a devastating loss of information.

LFR is well suited to this problem, as it produces excellent results starting with only about 10 cells worth of starting input genomic DNA, and even one single cell would provide enough DNA to perform LFR. The first step in LFR is generally low bias whole genome amplification, which can be of particular use in single cell genomic analysis. Due to DNA strand breaks and DNA losses in handling, even single molecule sequencing methods would likely require some level of DNA amplification from the single cell. The difficulty in sequencing single cells comes from attempting to amplify the entire genome. Studies performed on bacteria using MDA have suffered from loss of approximately half of the genome in the final assembled sequence with a fairly high amount of variation in coverage across those sequenced regions. This can partially be explained as a result of the initial genomic DNA having nicks and strand breaks which cannot be replicated at the ends and are thus lost during the MDA process. LFR provides a solution to this problem through the creation of long overlapping fragments of the genome prior to MDA. According to one embodiment of the invention, in order to achieve this, a gentle process is used to isolate genomic DNA from the cell. The largely intact genomic DNA is then be lightly treated with a frequent nickase, resulting in a semi-randomly nicked genome. The strand-displacing ability of phi29 is then used to polymerize from the nicks creating very long (>200 kb) overlapping fragments. These fragments are then be used as starting template for LFR.

### Base Calling, Mapping and Assembly

Data generated using any of the sequencing methods described herein can be analyzed and assembled using methods known in the art.

In some embodiments, four images, one for each color dye, are generated for each queried genomic position. The position of each spot in an image and the resulting intensities for each of the four colors is determined by adjusting for crosstalk between dyes and background intensity. A quantitative model can be fit to the resulting four-dimensional dataset. A base is called for a given spot, with a quality score that reflects how well the four intensities fit the model.

In further embodiments, read data is encoded in a compact binary format and includes both a called base and quality score. The quality score is correlated with base accuracy. Analysis software, including sequence assembly software, can use the score to determine the contribution of evidence from individual bases with a read.

Reads are generally “gapped” due to the DNB structure. Gap sizes vary (usually +/−1 base) due to the variability inherent in enzyme digestion. Due to the random-access nature of cPAL, reads may occasionally have an unread base (“no-call”) in an otherwise high-quality DNB. Read pairs are mated as described in further detail herein.

Mapping software capable of aligning read data to a reference sequence can be used to map data generated by the sequencing methods described herein. Such mapping software will generally be tolerant of small variations from a reference sequence, such as those caused by individual genomic variation, read errors, or unread bases. This property often allows direct reconstruction of SNPs. To support assembly of larger variations, including large-scale structural changes or regions of dense variation, each arm of a DNB can be mapped separately, with mate pairing constraints applied after alignment.

Assembly of sequence reads can in some embodiments utilize software that supports DNB read structure (mated, gapped reads with non-called bases) to generate a diploid genome assembly that can in some embodiments be leveraged off of sequence information generating LFR methods of the present invention for phasing heterozygote sites.

Methods of the present invention can be used to reconstruct novel segments not present in a reference sequence. Algorithms utilizing a combination of evidential (Bayesian) reasoning and de Bruijin graph-based algorithms may be used in some embodiments. In some embodiments, statistical models empirically calibrated to each dataset can be used, allowing all read data to be used without pre-filtering or data trimming. Large scale structural variations (including without limitation deletions, translocations, and the like) and copy number variations can also be detected by leveraging mated reads.

### EXAMPLES

### Example 1

### Producing DNBs

The following are exemplary protocols for producing DNBs (also referred to herein as “amplicons”) from nucleic acid templates of the invention comprising target nucleic acids interspersed with one or more adaptors. Single-stranded linear nucleic acid templates are first subjected to amplification with a phosphorylated 5′ primer and a biotinylated 3′ primer, resulting in a double-stranded linear nucleic acid templates tagged with biotin.

First, streptavidin magnetic beads were prepared by resuspending MagPrep-Streptavidin beads (Novagen Part. No. 70716-3) in 1× bead binding buffer (150 mM NaCl and 20 mM Tris, pH 7.5 in nuclease free water) in nuclease-free microfuge tubes. The tubes were placed in a magnetic tube rack, the magnetic particles were allowed to clear, and the supernatant was removed and discarded. The beads were then washed twice in 800 μl 1× bead binding buffer, and resuspended in 80 μl 1× bead binding buffer. Amplified nucleic acid templates (also referred to herein as “library constructs”) from the PCR reaction were brought up to 60 μl volume, and 20 μl 4× bead binding buffer was added to the tube. The nucleic acid templates were then added to the tubes containing the MagPrep beads, mixed gently, incubated at room temperature for 10 minutes and the MagPrep beads were allowed to clear. The supernatant was removed and discarded. The MagPrep beads (mixed with the amplified library constructs) were then washed twice in 800 μl 1× bead binding buffer. After washing, the MagPrep beads were resuspended in 80 μl 0.1 N NaOH, mixed gently, incubated at room temperature and allowed to clear. The supernatant was removed and added to a fresh nuclease-free tube. 4 μl 3M sodium acetate (pH 5.2) was added to each supernatant and mixed gently.

Next, 420 μl of PBI buffer (supplied with QIAprep PCR Purification Kits) was added to each tube, the samples were mixed and then were applied to QIAprep Miniprep columns (Qiagen Part No. 28106) in 2 ml collection tubes and centrifuged for 1 minutes at 14,000 rpm. The flow through was discarded, and 0.75 ml PE buffer (supplied with QIAprep PCR Purification Kits) was added to each column, and the column was centrifuged for an additional 1 minute. Again the flow through was discarded. The column was transferred to a fresh tube and 50 μl of EB buffer (supplied with QIAprep PCR Purification Kits) was added. The columns were spun at 14,000 for 1 minute to elute the single-stranded nucleic acid templates. The quantity of each sample was then measured.

Circularization of Single-Stranded Templates Using CircLigase:

First, 10 pmol of the single-stranded linear nucleic acid templates was transferred to a nuclease-free PCR tube. Nuclease free water was added to bring the reaction volume to 30 μl, and the samples were kept on ice. Next, 4 μl 10× CircLigase Reaction Buffer (Epicentre Part. No. CL4155K), 2 μl 1 mM ATP, 2 μl 50 mM MnCl₂, and 2 μl CircLigase (100 U/μl) (collectively, 4× CircLigase Mix) were added to each tube, and the samples were incubated at 60° C. for 5 minutes. Another 10 μl of 4× CircLigase Mix was added was added to each tube and the samples were incubated at 60° for 2 hours, 80° C. for 20 minutes, then 4° C. The quantity of each sample was then measured.

Removal of Residual Linear DNA from CircLigase Reactions by Exonuclease Digestion.

First, 30 μl of each CircLigase sample was added to a nuclease-free PCR tube, then 3 μl water, 4 μl 10× Exonuclease Reaction Buffer (New England Biolabs Part No. B0293S), 1.5 μl Exonuclease I (20 U/μl, New England Biolabs Part No. M0293L), and 1.5 μl Exonuclease III (100 U/μl, New England Biolabs Part No. M0206L) were added to each sample. The samples were incubated at 37° C. for 45 minutes. Next, 75 mM EDTA, pH 8.0 was added to each sample and the samples were incubated at 85° C. for 5 minutes, then brought down to 4° C. The samples were then transferred to clean nuclease-free tubes. Next, 500 μl of PN buffer (supplied with QIAprep PCR Purification Kits) was added to each tube, mixed and the samples were applied to QIAprep Miniprep columns (Qiagen Part No. 28106) in 2 ml collection tubes and centrifuged for 1 minute at 14,000 rpm. The flow through was discarded, and 0.75 ml PE buffer (supplied with QIAprep PCR Purification Kits) was added to each column, and the column was centrifuged for an additional 1 minute. Again the flow through was discarded. The column was transferred to a fresh tube and 40 μl of EB buffer (supplied with QIAprep PCR Purification Kits) was added. The columns were spun at 14,000 for 1 minute to elute the single-stranded library constructs. The quantity of each sample was then measured.

Circle Dependent Replication for DNB Production:

The nucleic acid templates were subjected to circle dependent replication to create DNBs comprising concatamers of target nucleic acid and adaptor sequences. 40 fmol of exonucleoase-treated single-stranded circles were added to nuclease-free PCR strip tubes, and water was added to bring the final volume to 10.0. μl. Next, 10 μl of 2× Primer Mix (7 μl water, 2 μl 10× phi29 Reaction Buffer (New England Biolabs Part No. B0269S), and 1 μl primer (2 μM)) was added to each tube and the tubes were incubated at room temperature for 30 minutes. Next, 20 μl of phi 29 Mix (14 μl water, 2 μl 10× phi29 Reaction Buffer (New England Biolabs Part No. B0269S), 3.2 dNTP mix (2.5 mM of each dATP, dCTP, dGTP and dTTP), and 0.8 μl phi29 DNA polymerase (10 U/μl, New England Biolabs Part No. M0269S)) was added to each tube. The tubes were then incubated at 30° C. for 120 minutes. The tubes were then removed, and 75 mM EDTA, pH 8.0 was added to each sample. The quantity of circle dependent replication product was then measured.

Determining DNB Quality:

Once the quantity of the DNBs was determined, the quality of the DNBs was assessed by looking at color purity. The DNBs were suspended in amplicon dilution buffer (0.8× phi29 Reaction Buffer (New England Biolabs Part No. B0269S) and 10 mM EDTA, pH 8.0), and various dilutions were added into lanes of a flowslide and incubated at 30° C. for 30 minutes. The flowslides were then washed with buffer and a probe solution containing four different random 12-mer probes labeled with Cy5, Texas Red, FITC or Cy3 was added to each lane. The flow slides were transferred to a hot block pre-heated to 30° C. and incubated at 30° C. for 30 minutes. The flow slides were then imaged using Imager 3.2.1.0 software. The quantity of circle dependent replication product was then measured.

### Example 2

### Single and Double c-PAL

Different lengths of fully degenerate second anchor probes were tested in a two anchor probe detection system. The combinations used were: 1) standard one anchor ligation using an anchor that binds to the adaptor adjacent to the target nucleic acid and a 9-mer sequencing probe, reading at position 4 from the adaptor 2) two anchor ligation using the same first anchor and a second anchor comprising a degenerate five-mer and a 9-mer sequencing probe, reading at position 9 from the adaptor; 3) two anchor ligation using the same first anchor and a second anchor comprising a degenerate six-mer and a 9-mer sequencing probe, reading at position 10 from the adaptor; and 4) two anchor ligation using the same first anchor and a second anchor comprising a degenerate eight-mer and a 9-mer sequencing probe, reading at position 12 from the adaptor. 1 μM of a first anchor probe and 6 μM of a degenerate second anchor probe were combined with T4 DNA ligase in a ligase reaction buffer and applied to the surface of the reaction slide for 30 minutes, after which time the unreacted probes and reagents were washed from the slide. A second reaction mix containing ligase and fluorescent probes of the type 5′ FI-NNNNNBNNN or 5′ FI-NNBNNNNNN 5′ FI-NNNBNNNNN 5′ FI-NNNNBNNNN was introduced. FI represents one of four fluorophores, N represents any one of the four bases A, G, C, or T introduced at random, and B represents one of the four bases A, G, C, or T specifically associated with the fluorophore. After ligation for 1 hr the unreacted probes and reagents were washed from the slide and the fluorescence associated with each DNA target was assayed.

We examined signal intensities associated with the different length degenerate second anchor probes in the systems, with intensities decreasing with increased second anchor probe length. The fit scores for such intensities also decreased with the length of the degenerate second anchor, but still generated reasonable fit scores through the base 10 read.

We then examined the effect of time using the one anchor probe method and the two anchor probe method. The standard anchor and degenerate five-mer were both used with a 9-mer sequencing probe to read positions 4 and 9 from the adaptor, respectively. Although the intensity levels differed more in the two anchor probe method, both the standard one anchor method and the two anchor probe methods at both times demonstrated comparable fit scores, each being over 0.8.

Effect of Degenerate Second Anchor Probe Length on Intensity and Fit Score:

Different combinations of first and second anchor probes with varying second anchor probe length and composition were used to compare the effect of the degenerate anchor probe on signal intensity and fit score when used to identify a base 5′ of the adaptor. Standard one anchor methods were compared to signal intensities and fit scores using two anchor probe methods with either partially degenerate probes having some region of complementarity to the adaptor, or fully degenerate second anchor probes. Degenerate second anchor probes of five-mers to nine-mers were used at one concentration, and two of these—the 6-mer and the seven-mer, were also tested at 4× concentration. Second anchor probes comprising two nucleotides of adaptor complementarity and different lengths of degenerate nucleotides at their 3′ end were also tested at the first concentration. Each of the reactions utilized a same set of four sequencing probes for identification of the nucleotide present at the read position in the target nucleic acid.

The combinations used in the experiments are as follows:
Reaction 1:1 μM of a 12 base first anchor probe
   No second anchor probe
   Read position: 2 nt from the adaptor end
Reaction 2: 1 μM of a 12 base first anchor probe
   20 μM of 5 degenerate base second anchor probe
   Read position: 7 nt from the adaptor end
Reaction 3: 1 μM of a 12 base first anchor probe
   20 μM of a 6 degenerate base second anchor probe
   Read position: 8 nt from the adaptor end
Reaction 4: 1 μM of a 12 base first anchor probe
   20 μM of a 7 degenerate base second anchor probe
   Read position: 9 nt from the adaptor end
Reaction 5: 1 μM of a 12 base first anchor probe
   20 μM of an 8 degenerate base second anchor probe
   Read position: 10 nt from the adaptor end
Reaction 6: 1 μM of a 12 base first anchor probe
   20 μM of a 9 degenerate base second anchor probe
   Read position: lint from the adaptor end
Reaction 7: 1 μM of a 12 base first anchor probe
   80 μM of a 6 degenerate base second anchor probe
   Read position: 8 nt from the adaptor end
Reaction 8: 1 μM of a 12 base first anchor probe
   80 μM of a 7 degenerate base second anchor probe
   Read position: 9 nt from the adaptor end
Reaction 9: 1 μM of a 12 base first anchor probe
   20 μM of a Ent second anchor probe (4 degenerate bases-2 known bases)
   Read position: 6 nt from the adaptor end
Reaction 10:1 μM of a 12 base first anchor probe
   20 μM of a 7 nt second anchor probe (5 degenerate bases-2 known bases)
   Read position: 7 nt from the adaptor end
Reaction 11: 1 μM of a 12 base first anchor probe
   20 μM of an 8 nt second anchor probe (6 degenerate bases-2 known bases)
   Read position: 8 nt from the adaptor end

In studies using different combinations of anchor probes and sequencing probes, the length of the degenerate second anchor probe was shown to be best using a six-mer, whether it was completely degenerate or partially degenerate. The signal intensities using a fully degenerate six-mer at a higher concentration showed signal intensities similar to that of the partially degenerate six-mer. All data had fairly good fit scores except one reaction that used the longest of the second anchors, which also displayed the lowest intensity scores of the reactions performed.

Effect of First Anchor Probe Length on Intensity and Fit Score:

Different combinations of first and second anchor probes with varying first anchor probe length were used to compare the effect of the first anchor probe length on signal intensity and fit score when used to identify a base 3′ of the adaptor. Standard one anchor methods were compared to signal intensities and fit scores using two anchor probe methods with either partially degenerate probes having some region of complementarity to the adaptor, or fully degenerate second anchor probes. Each of the reactions utilized a same set of four sequencing probes for identification of the nucleotide present at the read position in the target nucleic acid. The combinations used in the experiment are as follows:
Reaction 1:1 μM of a 12 base first anchor probe
   No second anchor probe
   Read position: 5 nt from the adaptor end
Reaction 2: 1 μM of a 12 base first anchor probe
   20 μM of 5 degenerate base second anchor probe
   Read position: 10 nt from the adaptor end
Reaction 3: 1 μM of a 10 base first anchor probe
   20 μM of a 7 nt second anchor probe (5 degenerate bases-2 known bases)
   Read position: 10 nt from the adaptor end
Reaction 4: 1 μM of a 13 base first anchor probe
   20 μM of a 7 degenerate base second anchor probe
   Read position: 12 nt from the adaptor end
Reaction 5: 1 μM of a 12 base first anchor probe
   20 μM of an 7 degenerate base second anchor probe
   Read position: 12 nt from the adaptor end
Reaction 6: 1 μM of a 11 base first anchor probe
   20 μM of a 7 degenerate base second anchor probe
   Read position: 12 nt from the adaptor end
Reaction 7: 1 μM of a 10 base first anchor probe
   20 μM of a 7 degenerate base second anchor probe
   Read position: 12 nt from the adaptor end
Reaction 8: 1 μM of a 9 base first anchor probe
   80 μM of a 7 degenerate base second anchor probe
   Read position: 12 nt from the adaptor end

The signal intensity and fit scores observed show an optimum intensity resulting from use of the longer first anchor probes, which in part may be due to the greater meting temperature the longer probes provide to the combined anchor probe.

Effect of Kinase Incubations on Intensity and Fit Score Using Two Anchor Primer Methods:

The reactions as described above were performed at different temperatures using 1 μM of a 10 base first anchor probe, 20 μM of a 7-mer second anchor probe, and sequencing probe with the structure Fluor-NNNNBNNNN to read position 10 from the adaptor in the presence of a kinase at 1 Unit/ml for a period of three days. A reaction with a 15-mer first anchor and the sequencing probe served as a positive control. Although the kinase did have an effect on signal intensities as compared to the control, the range did not change from 4° C. to 37° C., and fit scores remained equivalent with the control. The temperature at which the kinase incubation did have an impact is 42° C., which also displayed a poor fit with the data.

The minimum time needed to kinase was then examined using the same probes and conditions as described above. Kinase incubation of five minutes or above resulting in effectively equivalent signal intensities and fit score.

### Example 3

### Human Genome Sequencing Using Unchained Base Reads on Self-Assembling DNA

Three human genomes were sequenced, generating an average of 45- to 87-fold coverage per genome and identifying 3.2-4.5 million sequence variants per genome. Validation of one genome dataset demonstrated a sequence accuracy of about 1 false variant per 100 kilobases.

Generation of Template Sequencing Substrates

Sequencing substrates were generated by means of genomic DNA fragmentation and recursive cutting with type IIS restriction enzymes and directional adaptor insertion as discussed herein. The four-adaptor library construction process resulted in: (i) high yield adaptor ligation and DNA circularization with minimal chimera formation, (ii) directional adaptor insertion with minimal creation of structures containing undesired adaptor topologies, (iii) iterative selection of constructs with desired adaptor topologies by PCR, (iv) efficient formation of strand-specific ssDNA circles, and (v) single tube solution-phase amplification of ssDNA circles to generate discrete (non-entangled) DNA nanoballs (DNBs) in high concentration. Although the process involved many independent enzymatic steps, it was largely recursive in nature and was amenable to automation for the processing of 96 sample batches.

Genomic DNA (“gDNA”) was fragmented by sonication to a mean length of 500 basepairs (“bp”), and fragments migrating within a 100 bp range (e.g. ˜400 to ˜500 bp for NA19240) were isolated from a polyacrylamide gel and recovered by QiaQuick column purification (Qiagen, Valencia, Calif.). Approximately 1 μg (˜3 pmol) of fragmented gDNA was treated for 60 minutes at 37° C. with 10 units of FastAP (Fermentas, Burlington, ON, CA), purified with AMPure beads (Agencourt Bioscience, Beverly, Mass.), incubated for 1 h at 12° C. with 40 units of T4 DNA polymerase (New England Biolabs (NEB), Ipswich, Mass.), and AMPure purified again, all according to the manufacturers' recommendations, to create non-phosphorylated blunt termini. The end-repaired gDNA fragments were then ligated to synthetic adaptor 1 (Ad1) arms according to the nick translation ligation process as described herein, which produced efficient adaptor-fragment ligation with minimal fragment-fragment and adaptor-adaptor ligation. Oligonucleotides used in adaptor construction and insertion according to the present invention were purchased from IDT. Palindromes were included to enhance formation of compact DNBs via 14-base intramolecular hybridization.

Approximately 1.5 pmol of end repaired gDNA fragments were incubated for 120 minutes at 14° C. in a reaction containing 50 mM Tris-HCl (pH 7.8), 5% PEG 8000, 10 mM MgCl2, 1 mM rATP, a 10-fold molar excess of 5′-phosphorylated and 3′ dideoxy terminated Ad1 arms and 4,000 units of T4 DNA ligase (Enzymatics, Beverly, Mass.). T4 DNA ligation of 5′PO₄ Ad1 arm termini to 3′OH gDNA termini produced a nicked intermediate structure, where the nicks consisted of dideoxy (and therefore non-ligatable) 3′ Ad1 arm termini and non-phosphorylated (and therefore non-ligatable) 5′ gDNA termini. After AMPure purification to remove unincorporated Ad1 arms, the DNA was incubated for 15 min at 60° C. in a reaction containing 200 μM Ad1 PCR1 primers, 10 mM Tris-HCl (pH 7.3), 50 mM KCl, 1.5 mM MgCl2, 1 mM rATP, 100 μM dNTPs, to exchange 3′ dideoxy terminated Ad1 oligos with 3′OH terminated Ad1 PCR1 primers. The reaction was then cooled to 37° C. and, after addition of 50 units of Taq DNA polymerase (NEB) and 2000 units of T4 DNA ligase, was incubated a further 30 minutes at 37° C., to create functional 5′PO₄ gDNA termini by Taq-catalyzed nick translation from Ad1 PCR1 primer 3′ OH termini, and to seal the resulting repaired nicks by T4 DNA ligation.

Approximately 700 pmol of AMPure purified Ad1-ligated material was subjected to PCR (6-8 cycles of 95° C. for 30 seconds, 56° C. for 30 seconds, 72° C. for 4 minutes) in a 800 μL reaction consisting of 40 units of PfuTurbo Cx (Stratagene, La Jolla, Calif.) 1×Pfu Turbo Cx buffer, 3 mM MgSO4, 300 μM dNTPs, 5% DMSO, 1M Betaine, and 500 nM each Ad1 PCR1 primer. This process resulted in selective amplification of the ˜350 fmol of template containing both left and right Ad1 arms, to produce approximately 30 pmol of PCR product incorporating dU moieties at specific locations within the Ad1 arms. Approximately 24 pmol of AMPure-purified product was treated at 37° C. for 60 minutes with 10 units of a UDG/EndoVIII cocktail (USER; NEB) to create Ad1 arms with complementary 3′ overhangs and to render the right Ad1 arm-encoded AcuI site partially single-stranded. This DNA was incubated at 37° C. for 12 hours in a reaction containing 10 mM Tris-HCl (pH 7.5), 50 mM NaCl, 1 mM EDTA, 50 μM s-adenosyl-L-methionine, and 50 units of Eco571 (Fermentas, Glen Burnie, Md.), to methylate the left Ad1 arm AcuI site as well as genomic AcuI sites. Approximately 18 pmol of AMPure-purified, methylated DNA was diluted to a concentration of 3 nM in a reaction consisting of 16.5 mM Tris-OAc (pH 7.8), 33 mM KOAc, 5 mM MgOAc, and 1 mM ATP, heated to 55° C. for 10 min, and cooled to 14° C. for 10 min, to favor intramolecular hybridization (circularization).

The reaction was then incubated at 14° C. for 2 hours with 3600 units of T4 DNA ligase (Enzymatics) in the presence of 180 nM of non-phosphorylated bridge oligo to form monomeric dsDNA circles containing top-strand-nicked Ad1 and double-stranded, unmethylated right Ad1 AcuI sites. The Ad1 circles were concentrated by AMPure purification and incubated at 37° C. for 60 minutes with 1000 PlasmidSafe exonuclease (Epicentre, Madison, Wis.) according to the manufacturer's instructions, to eliminate residual linear DNA.

Approximately 12 pmol of Ad1 circles were digested at 37° C. for 1 hour with 30 units of AcuI (NEB) according to the manufacturer's instructions to form linear dsDNA structures containing Ad1 flanked by two segments of insert DNA. After AMPure purification, approximately 5 pmol of linearized DNA was incubated at 60° C. for 1 hour in a reaction containing 10 mM Tris-HCl (pH8.3), 50 mM KCl, 1.5 mM MgCl2, 0.163 mM dNTP, 0.66 mM dGTP, and 40 units of Taq DNA polymerase (NEB), to convert the 3′ overhangs proximal to the active (right) Ad1 AcuI site to 3′G overhangs by translation of the Ad1 top-strand nick. The resulting DNA was incubated for 2 hours at 14° C. in a reaction containing 50 mM Tris-HCl (pH 7.8), 5% PEG 8000, 10 mM MgCl2, 1mM rATP, 4000 units of T4 DNA ligase, and a 25-fold molar excess of asymmetric Ad2 arms, with one arm designed to ligate to the 3′ G overhang, and the other designed to ligate to the 3′ NN overhang, thereby yielding directional (relative to Ad1) Ad2 arm ligation. Approximately 2 pmol of Ad2-ligated material was purified with AMPure beads, PCR-amplified with PfuTurbo Cx and dU-containing Ad2-specific primers, AMPure purifies, treated with USER, circularized with T4 DNA ligase, concentrated with AMPure and treated with PlasmidSafe, all as above, to create Ad1+2-containing dsDNA circles.

Approximately 1 pmol of Ad1+2 circles were PCR-amplified with Ad1 PCR2 dU-containing primers, AMPure purified, and USER digested, all as discussed above, to create fragments flanked by Ad1 arms with complimentary 3′ overhangs and to render the left Ad1 AcuI site partially single-stranded. The resulting fragments were methylated to inactivate the right Ad1 AcuI site as well as genomic AcuI sites, AMPure purified and circularized, all as above, to form dsDNA circles containing bottom strand-nicked Ad1 and double stranded unmethylated left Ad1 AcuI sites. The circles were concentrated by AMPure purification, AcuI digested, AMPure purified G-tailed, and ligated to asymmetric Ad3 arms, all as discussed above, thereby yielding directional Ad3 arm ligation. The Ad3-ligated material was AMPure purified, PCR-amplified with dU-containing Ad3-specific primers, AMPure purified, USER-digested, circularized and concentrated, all as above, to create Ad1+2+3-containing circles, wherein Ad2 and Ad3 flank Ad1 and contain EcoP15 recognition sites at their distal termini.

Approximately 10 pmol of Ad1+2+3 circles were digested for 4 hours at 37° C. with 100 units of EcoP15 (NEB) according to the manufacturer's instructions, to liberate a fragment containing the three adaptors interspersed between four gDNA fragments. After AMPure purification, the digested DNA was end-repaired with T4 DNA polymerase as above, AMPure purified as above, incubated for 1 hour at 37° C. in a reaction containing 50 mM NaCl, 10 mM Tris-HCl (pH7.9), 10 mM MgCl2, 0.5 mM dATP, and 16 units of Klenow exo-(NEB) to add 3′ A overhangs, and ligated to T-tailed Ad4 arms as above. The ligation reaction was run on a polyacrylamide gel, and Ad1+2+3+Ad4-arm-containing fragments were eluted from the gel and recovered by QiaQuick purification. Approximately 2 pmol of recovered DNA was amplified as above with Pfu Turbo Cx (Stratagene) plus a 5′-biotinylated primer specific for one Ad4 arm and a 5′PO₄ primer specific for the other Ad4 arm.

Approximately 25 pmol of biotinylated PCR product was captured on streptavidin-coated, Dynal paramagnetic beads (Invitrogen, Carlsbad, Calif.), and the non-biotinylated strand, which contained one 5′ Ad4 arm and one 3′ Ad4 arm, was recovered by denaturation with 0.1 N NaOH, all according to the manufacturer's instructions. After neutralization, strands containing Ad1+2+3 in the desired orientation with respect to the Ad4 arms were purified by hybridization to a three-fold excess of an Ad1 top strand-specific biotinylated capture oligo, followed by capture on streptavidin beads and 0.1 N NaOH elution, all according to the manufacturer's instructions. Approximately 3 pmol of recovered DNA was incubated for 1 hour at 60° C. with 200 units of CircLigase (Epicentre) according to manufacturer's instructions, to form single-stranded (ss)DNA Ad1+2+3+4-containing circles, and then incubated for 30 minutes at 37° C. with 100 units of ExoI and 300 units of ExoIII (both from Epicenter) according to the manufacturer's instructions, to eliminate non-circularized DNA.

To assess representational biases during circle construction, genomic DNA and intermediate steps in the library construction process were assayed by quantitative PCR (QPCR) with the StepOne platform (Applied Biosystems, Foster City, Calif.) and a SYBR Green-based QPCR assay (Quanta Biosciences, Gaithersburg, Md.) for the presence and concentration of a set of 96 dbSTS markers representing a range of locus GC contents. The markers were selected from dbSTS to be less than 100 bp in length, to use primers 20 bases in length and with GC content of 45-55%, and to represent a range of locus GC contents. Start and stop coordinates are from NCBI Build 36. Amplicon GC contents were of the amplified PCR product, and 1 kb GC contents were calculated from the 1 kb interval centered on the amplicons. Raw cycle threshold (Ct) values were collected for each marker in each sample. Next, the mean Ct for each sample was subtracted from its respective raw Ct values to generate a set of normalized Ct values, such that the mean normalized Ct value for each sample was zero. Finally, the mean (from four replicate runs) normalized Ct of each marker in gDNA was subtracted from its respective normalized Ct values, to produce a set of delta Ct values for each marker in each sample. This analysis revealed an increase in the concentration of higher GC content markers at the expense of higher AT content markers in the Ad1, Ad2, and Ad3 circles relative to genomic DNA. On average, there was a 1.4 Ct (2.5-fold) difference in concentrations of loci with 1 kb GC content of 30-35% versus those of 50-55%. This bias was similar to the fragment and base level coverage bias observed in the mapped cPAL data.

To assess library construct structure, 4Ad hybrid-captured, single-stranded library DNA was PCR-amplified with Taq DNA polymerase (NEB) and Ad4-specific PCR primers. These PCR products were cloned with the TopoTA cloning kit (Invitrogen), and colony PCR was used to generate PCR amplicons from 192 independent colonies. These PCR products were purified with AMPure beads and sequence information was collected from both strands with Sanger dideoxy sequencing (MCLAB, South San Francisco, Calif.). The resulting traces were filtered for high quality data, and clones containing a library insert with at least one good read were included in the analysis. Table 1 shows data from Sanger sequencing of library intermediates to assess adaptor structure. 147 of 192 library clones contained at least one high quality Sanger read. 143 of these 147 clones (>97%) contained all 4 adaptors in the expected orientation and order. Moreover, 3 of the 4 clones (*) with aberrant adaptor structure were expected to be eliminated from the library during the RCR reaction used to generate DNBs, implying about 99% of DNBs were expected to have the correct adaptor structure. Data derived from NA07022

TABLE 1

| | # clones | % of clones |
|---|---|---|
| All adaptors intact | 143 | 97.2 |
| Adaptor 2 missing | 1 | 0.7 |
| Adaptor 1, 2, 3 missing* | 1 | 0.7 |
| Adaptor 1, 2, 3 wrong orientation* | 2 | 1.4 |
| Total | 147 | 100.0 |

Table 2 shows results from Sanger sequencing of library intermediates to identify adaptor mutations. Analysis of 89 cloned library constructs for which high quality forward and reverse Sanger sequencing data was available revealed about one mutation per 1000 bp of adaptor sequence. Also, 5 of the 89 cloned library constructs (5.6%) had mutations within 10 bp of one of its eight adaptor termini; such mutations might be expected to affect cPAL data quality. The majority of the adaptor mutations were likely introduced by errors in oligonucleotides synthesis. A much lower mutation rate would be expected to result from 32 cycles of high fidelity PCR (32*1.3E-6<1 in 10,000 bp). Data derived from NA07022.

TABLE 2

| | | | | Mutations in: | | | Mu- |
|---|---|---|---|---|---|---|---|
| | | | | Adaptor | Other | All | tation |
| Adaptor | bp | # clones | Total bp | termini | region | regions | rate |
| 1 | 44 | 89 | 3916 | 3 | 2 | 5 | 0.13% |
| 2 | 56 | 89 | 4984 | 2 | 4 | 6 | 0.12% |
| 3 | 56 | 89 | 4984 | 0 | 5 | 5 | 0.10% |
| 4 | 66 | 89 | 9523 | 0 | 8 | 8 | 0.08% |
| Total | 222 | 89 | 23407 | 5 | 19 | 24 | 0.10% |

Generation of DNBs

The circles generated according to the above described method were replicated with Phi29 polymerase. Using a controlled, synchronized synthesis hundreds of tandem copies of the sequencing substrate were obtained in palindrome-promoted coils of single stranded DNA, referred to herein as DNA nanoballs (DNBs). 100 fmol of Ad1+2+3+4 ssDNA circles were incubated for 10 minutes at 90° C. in a 400 μL reaction containing 50 mM Tris-HCl (pH 7.5), 10 mM (NH₄)₂SO₄, 10 mM MgCl₂, 4 mM DTT, and 100 nM Ad4 PCR 5B primer. The reaction was adjusted to an 800 μL reaction containing the above components plus 800 μM each dNTP and 320 units of Phi29 DNA polymerase (Enzymatics), and incubated for 30 min at 30° C. to generate DNBs. Short palindromes in the adaptors promote coiling of ssDNA concatamers via reversible intra-molecular hybridization into compact ˜300 nm DNBs, thereby avoiding entanglement with neighboring DNBs (also referred to herein as “replicons”). The combination of synchronized rolling circle replication (RCR) conditions and palindrome-driven DNB assembly generated over 20 billion discrete DNBs/ml of RCR reaction. These compact structures were stable for several months without evidence of degradation or entanglement.

Generation of Random Arrays of DNBs

The DNBs were adsorbed onto photolithographically etched, surface modified 25×75 mm silicon substrates with grid-patterned arrays of ˜300 nm spots for DNB binding. The use of the grid-patterned surfaces increased DNA content per array and image information density relative to arrays formed on surfaces without such patterns. These arrays are random arrays, in that it is not known which sequences are located at each point of the array until the sequencing reactions are conducted.

To manufacture patterned substrates, a layer of silicon dioxide was grown on the surface of a standard silicon wafer (Silicon Quest International, Santa Clara, Calif.). A layer of titanium was deposited over the silicon dioxide, and the layer was patterned with fiducial markings with conventional photolithography and dry etching techniques. A layer of hexamethyldisilizane (HMDS) (Gelest Inc., Morrisville, Pa.) was added to the substrate surface by vapor deposition, and a deep-UV, positive-tone photoresist material was coated to the surface by centrifugal force. Next, the photoresist surface was exposed with the array pattern with a 248 nm lithography tool, and the resist was developed to produce arrays having discrete regions of exposed HMDS. The HMDS layer in the holes was removed with a plasma-etch process, and aminosilane was vapor-deposited in the holes to provide attachment sites for DNBs. The array substrates were recoated with a layer of photoresist and cut into 75 mm×25 mm substrates, and all photoresist material was stripped from the individual substrates with ultrasonication. Next, a mixture of 50 μm polystyrene beads and polyurethane glue was applied in a series of parallel lines to each diced substrate, and a coverslip was pressed into the glue lines to form a six-lane gravity/capillary-driven flow slide. The aminosilane features patterned onto the substrate serve as binding sites for individual DNBs, whereas the HMDS inhibits DNB binding between features.

DNBs were loaded into flow slide lanes by pipetting 2- to 3-fold more DNBs than binding sites on the slide. Loaded slides were incubated for 2 hours at 23° C. in a closed chamber, and rinsed to neutralize pH and remove unbound DNBs.

Sequencing Reactions

Cell lines derived from two individuals previously characterized by the HapMap project, a Caucasian male of European decent (NA07022) and a Yoruban female (NA19240), were sequenced. In addition, lymphoblast DNA from a Personal Genome Project Caucasian male sample, PGP1 (NA20431) was sequenced. Automated cluster analysis of the four-dimensional intensity data produced raw base reads and associated raw base scores.

High-accuracy cPAL sequencing chemistry was used to independently read up to 10 bases adjacent to each of eight anchor sites, resulting in a total of 31- to 35-base mate-paired reads (62 to 70 bases per DNB). cPAL is an unchained hybridization and ligation technology that extends conventional sequencing by ligation reactions using degenerate anchors, providing extended read lengths (e.g. 8-15 bases) adjacent to each of the eight inserted adaptor sites with similar accuracy at all read positions. There are 70 sequenced positions within one DNB. Read positions of up to 10 bases from an adaptor were detected. Discordance was determined by mapping reads to the reference (taking the best match in cases where multiple reasonable hits were found) and tallying disagreements between the read and the reference at each position. Unchained base reading tolerates sporadic base detection failures in otherwise good reads. The majority of errors occur in a small fraction of low quality bases. Data derived from NA07022. In general, approximately 10 bases adjacent to each adaptor could be read using the cPAL technology.

Unchained sequencing of target nucleic acids by combinatorial probe anchor ligation (cPAL) involves detection of ligation products formed by an anchor oligonucleotide hybridized to part of an adaptor sequence, and a fluorescent degenerate sequencing probe that contains a specified nucleotide at an “interrogation position”. If the nucleotide at the interrogation position is complementary to the nucleotide at the detection position within the target, ligation is favored, resulting in a stable probe-anchor ligation product that can be detected by fluorescent imaging.

Four fluorophores were used to identify the base at an interrogation position within a sequencing probe, and pools of four sequencing probes were used to query a single base position per hybridization-ligation-detection cycle. For example, to read position 4, 3′ of the anchor, the following 9-mer sequencing probes were pooled where “p” represents a phosphate available for ligation and “N” represents degenerate bases:

5′-pNNNANNNNN-Quasar 670
5′-pNNNGNNNNN-Quasar 570
5′-pNNNCNNNNN-Cal fluor red 610
5′-pNNNTNNNNN-fluorescein

A total of forty probes were synthesized (Biosearch Technologies, Novato, Calif.) and HPLC-purified with a wide peak cut. These probes consisted of five sets of four probes designed to query positions 1 through 5 5′ of the anchor and five sets of four probes designed to query positions 3′ of the anchor. These probes were pooled into 10 pools, and the pools were used in combinatorial ligation assays with a total of 16 anchors [4 adaptors×2 adaptor termini×2 anchors (standard and extended)], hence the name combinatorial probe-anchor ligation (cPAL).

To read positions 1-5 in the target sequence adjacent to the adaptor, 1 μM anchor oligo was pipetted onto the array and hybridized to the adaptor region directly adjacent to the target sequence for 30 minutes at 28° C. A cocktail of 1000 U/ml T4 DNA ligase plus four fluorescent probes (at typical concentrations of 1.2 μM T, 0.4 μM A, 0.2 μM C, and 0.1 μM G) was then pipetted onto the array and incubated for 60 minutes at 28° C. Unbound probe was removed by washing with 150 mM NaCl in Tris buffer pH 8.

In general, T4 DNA ligase will ligate probes with higher efficiency if they are perfectly complementary to the regions of the target nucleic acid to which they are hybridized, but the fidelity of ligase decreases with distance from the ligation point. To minimize errors due to incorrect pairing between a sequencing probe and the target nucleic acid, it is useful to limit the distance between the nucleotide to be detected and the ligation point of the sequencing and anchor probes. By employing extended anchors capable of reaching 5 bases into the unknown target sequence, it was possible to use T4 DNA ligase to read positions 6-10 in the target sequence.

Creation of extended anchors involved ligation of two anchor oligos designed to anneal next to each other on the target DNB. First-anchor oligos were designed to terminate near the end of the adaptor, and second-anchor oligos, comprised in part of five degenerate positions that extended into the target sequence, were designed to ligate to the first anchor. In addition, degenerate second-anchor oligos were selectively modified to suppress inappropriate (e.g., self) ligation. For assembly of 3′ extended anchors (which contribute their 3′ ends to ligation with sequencing probe), second-anchor oligos were manufactured with 5′ and 3′ phosphate groups, such that 5′ ends of second-anchors could ligate to 3′ ends of first-anchors, but 3′ ends of second-anchors were unable to participate in ligation, thereby blocking second-anchor ligation artifacts. Once extended anchors were assembled, their 3′ ends were activated by dephosphorylation with T4 polynucleotide kinase (Epicentre). Similarly, for assembly of 5′ extended anchors (which contribute their 5′ ends to ligation with sequencing probe), first-anchors were manufactured with 5′ phosphates, and second-anchors were manufactured with no 5′ or 3′ phosphates, such that the 3′ end of second-anchors could ligate to 5′ ends of first-anchors, but 5′ ends of second-anchors were unable to participate in ligation, thereby blocking second-anchor ligation artifacts. Once extended anchors were assembled, their 5′ ends were activated by phosphorylation with T4 polynucleotide kinase (Epicentre).

First-anchors (4 μM) were typically 10 to 12 bases in length and second-anchors (24 μM) were 6 to 7 bases in length, including the five degenerate bases. The use of high concentrations of second-anchor introduced negligible noise and minimal cost relative to the alternative of using high concentrations of labeled probes. Anchors were ligated with 200 U/ml T4 DNA ligase at 28° C. for 30 minutes and then washed three times before addition of 1 U/ml T4 polynucleotide kinase (Epicentre) for 10 minutes. Sequencing of positions 6-10 then proceeded as above for reading positions 1-5.

After imaging, the hybridized anchor-probe conjugates were removed with 65% formamide, and the next cycle of the process was initiated by the addition of either single-anchor hybridization mix or two-anchor ligation mix. Removal of the probe-anchor product is an important feature of unchained base reading. Starting a new ligation cycle on the clean DNA allows accurate measurements at 20 to 30% ligation yield, which can be achieved at low cost and high accuracy with low concentrations of probes and ligase.

Imaging

A Tecan (Durham N.C.) MSP 9500 liquid handler was used for automated cPAL biochemistry, and a robotic arm was used to interchange the slides between the liquid handler and an imaging station. The imaging station consisted of a four-color epi-illumination fluorescence microscope built with off-the-shelf components, including an Olympus (Center Valley, Pa.) NA=0.95 water-immersion objective and tube lens operated at 25-fold magnification; Semrock (Rochester, N.Y.) dual-band fluorescence filters, FAM/Texas Red and CY3/CY5; a Wegu (Markham, Ontario, Canada) autofocus system; a Sutter (Novato Calif.) 300W xenon arc lamp coupled to Lumatec (Deisenhofen, Germany) 380 liquid light guide; an Aerotech (Pittsburgh, Pa.) ALS130 X-Y stage stack; and two Hamamatsu (Bridgewater, N.J.) 9100 1-megapixel EM-CCD cameras. Each slide was divided into 6,396 320 μm×320 μm fields. The fields were organized into six 1066-field groups, corresponding to the lanes created by glue lines on the substrate. Four-color images of each group were generated (requiring one filter change) before moving to the next group. Images were taken in step-and-repeat mode at an effective rate of seven frames per second. To maximize microscope utilization and match the biochemistry cycle time and imaging cycle time, six slides were processed in parallel with staggered biochemistry start times, such that the imaging of slide N was completed just as slide N+1 was completing its biochemistry cycle.

Further embodiments may include continuous imaging, which will generate a 30-fold throughput improvement to 250 Gb per instrument day and over 1 Tb per instrument day with further camera improvements.

Base Calling

Each imaging field contained 225×225=50625 spots or potential DNB features. The four images associated with a field were processed independently to extract DNB intensity information, with the following steps: (1) background removal, (2) image registration, and (3) intensity extraction. First, background was estimated with a morphological opening (erosion followed by dilation) operation. The resulting background image was then subtracted from the original image. Next, a flexible grid was registered to the image. In addition to correction for rotation and translation, this grid allowed for (R−1)+(C−1) degrees (here: R=C=225) of freedom for scale/pitch, where R and C are the number of DNB rows and columns, respectively, such that each row or column of the grid was allowed to float slightly in order to find the optimal fit to the DNB array. This process accommodates optical aberrations in the image as well as fractional pixels per DNB. Finally, for each grid point, a radius of one pixel was considered; and within that radius, the average of the top three pixels was computed and returned as the extracted intensity value for that DNB.

The data from each field were then subjected to base calling, which involved four major steps: (1) crosstalk correction, (2) normalization, (3) calling bases, and (4) raw base score computation. First, crosstalk correction was applied to reduce optical (fixed) and biochemical (variable) crosstalk between the four channels. All the parameters—fixed or variable—were estimated from the data for each field. A system of four intercepting lines (at one point) was fit to the four-dimensional intensity data with a constrained optimization algorithm. Sequential quadratic programming and genetic algorithms were used for the optimization process. The fit model was then used to reverse-transform the data into the canonical space. After crosstalk correction, each channel was independently normalized, with the distribution of the points on the corresponding channel. Next, the axis closest to each point was selected as its base call. Bases were called on all spots regardless of quality. Each spot then received a raw base score, reflecting the confidence level in that particular base call. The raw base score computation was made by the geometrical mean of several sub-scores, which capture the strength of the clusters as well as their relative position and spread and the position of the data point within its cluster.

DNB Mapping and Sequence Assembly

The sequence reads were mapped to the human genome reference assembly using methods known in the art and as described in 61/173,967, filed Apr. 29, 2009, which is herein incorporated by reference in its entirety for all purposes and in particular for all teachings related to assembly of sequences and mapping of sequences to reference sequences. Assembly and mapping of the sequence reads resulted in about 124 to about 241 Gb mapped and an overall genome coverage of approximately 45- to 87-fold per genome.

The gapped read structure of the present invention requires some adjustments to standard informatic analyses. It is possible to represent each arm as a continuous string of bases if one fixes the lengths of the gaps between reads (e.g. with the most common values), replaces positive gaps with Ns, and uses a consensus call for base positions where reads overlap. Such a string can be aligned to a reference sequence using dynamic programming including standard Smith-Waterman local alignment scoring, or with modified scoring schemes that allow indels only at the locations of gaps between reads. Methods for high-speed mapping of short reads involving some form of indexing of the reference genome can also be applied, though indexes relying on ungapped seeds longer than 10 bases limit the portion of the arm that can be compared to the index and/or require limits on the allowed gap sizes. In simulations, we have found that missing the correct gap structure for even a small fraction (<1%) of arms can substantially increase variation calling errors, because we miss the correct alignment for these arms and may thus put too much confidence in a false mapping with the wrong gap structure. Consequently, the present invention provides a method for efficient mapping of DNBs that can find nearly all correct mappings.

Mate-paired arm reads were aligned to the reference genome in a two-stage process. First, left and right arms were aligned independently using indexing of the reference genome. This initial search will find all locations in the genome that match the arm with at most two single-base substitutions, but may find some locations that have up to five mismatches. The number of mismatches in the reported alignments was further limited so that the expectation of finding an alignment to random sequence of the same length as the reference was <4⁻³. If a particular arm had more than 1.000 alignments, no alignments were carried forward, and the arm was marked as “overflow”. Second, for every location of a left arm identified in the first stage, the right arm was subjected to a local alignment process, which was constrained to a genomic interval informed by the distribution of the mate distance (here, 0 to 700 bases away). Up to four single-base mismatches were allowed during this process; the number of mismatches was further limited so that the expectation of a random alignment of the entire mate pair was <4⁻⁷. The same local search for the left arms was performed in the vicinity of right arm alignments.

At both stages, the alignment of a gapped arm read was performed by trying multiple combinations of gap values. The frequencies of gap values were estimated for every library by aligning a sample of arm reads from that library with lenient limits on the gap values. During the bulk alignment, only a subset of the gap values was used for performance reasons; the cumulative frequency of the neglected gap values was approximately 10⁻³. Both stages were capable of aligning arms containing positions that were not sequenced successfully (no-calls). The expectation calculations above take into account the number of no-calls in the arm. Finally, if a mate-pair had any consistent locations of arms (that is, left and right arms were on the same strand, in the proper order and within the expected mate-distance distribution), then only these locations were retained. Otherwise, all locations of the mate-pair were retained. In either case, for performance reasons, at most 50 locations for every arm were reported; arms that had more retained locations were marked as “overflow”, and no locations were reported. The overall data yield of spots imaged through mapped reads varied between 40 and 50% reflecting end-to-end losses from all process inefficiencies including unoccupied array spots, low quality areas, abnormal DNBs and DNBs with non-human (e.g. EBV-derived) DNA.

The genome sequence was assembled from reads using methods known in the art and described herein. The assembled sequence was then compared to reference sequences for confirmation.

The assembled genome datasets were subjected to a routine identity QC analysis protocol to confirm their sample of origin. Assembly-derived SNP genotypes were found to be highly concordant with those independently obtained from the original DNA samples, indicating the dataset was derived from the sample in question. Also, mitochondrial genome coverage in each lane was sufficient to support lane-level mitochondrial genotyping (average of 31-fold per lane). A 39-SNP mitochondrial genotype profile was compiled for each lane, and compared to that of the overall dataset, demonstrating that each lane derived from the same source.

This and mapped coverage showed a substantial deviation from Poisson expectation but only a small fraction of bases had insufficient coverage. For each sample, coverage of the least covered 10% of the genome varied between approximately 13-fold and 22-fold. Much of this coverage bias was accounted for by local GC content in NA07022, a bias that was significantly reduced by improved PCR conditions in NA19240. The distributions were normalized for facile comparison. The distribution for Poisson sampling of reads, and for mapping with simulated 400 bp mate-pair DNB reads are provided for comparison. In NA19240 only a few percent of the mappable genome is more than 3-fold underrepresented or more than two-fold overrepresented. The percent coverage of genome for NA20431 was similar to NA07022. The principal difference between these two libraries is in the conditions used for PCR. NA19240 was amplified using conditions described in SOM, above. In contrast, NA07022 was amplified using twice the amount of DMSO and Betaine as was used for NA19240, resulting in overrepresentation of high GC content regions of the genome. Single-allele calls (one alternate allele, one no-called allele) were considered detected if they passed the call threshold.

Discordance with respect to the reference genome in uniquely mapping reads from NA07022 was 2.1% (with a range of about 1.4%-3.3% per slide). However, considering only the highest scoring 85% of base calls reduced the raw read discordance to 0.47% including true variant positions.

A range of 2.91 to 4.04 million SNPs was identified with respect to the reference genome, 81 to 90% of which are reported in dbSNP, as well as short indels and block substitutions. With the use of local de novo assembly methods, indels were detected in sizes ranging up to 50 bp. As expected, indels in coding regions tended to occur in multiples of length 3, indicating the possible selection of minimally impacting variants in coding regions.

As an initial test of sequence accuracy, the called SNPs generated according to the method described above were compared with the HapMap phase I/II SNP genotypes reported for NA07022. The present method fully called 94% of these positions with an overall concordance of 99.15% (the remaining 6% of positions were either half-called or not called).

Furthermore, 96% of the Infinium (Illumina, San Diego, Calif.) subset of the HapMap SNPs were fully called with an overall concordance rate of 99.88%, reflecting the higher reported accuracy of these genotypes. Similar concordance rates with available SNP genotypes were observed in NA19240 (with a call rate of over 98%) and NA20431.

Because the whole-genome false positive rate cannot be accurately estimated from known SNP loci, a random subset of novel non-synonymous variants in NA07022 were tested, because this category is enriched for errors. Error rates were extrapolated from the targeted sequencing of 291 such loci, and the false positive rate was estimated at about one variant per 100 kb, including approximately 6.1 substitution variants, approximately 3.0 short deletion variants, approximately 3.9 short insertion variants and approximately 3.1 block variants per Mb. (Table 3).

TABLE 3

| | | | | | Estimated | |
|---|---|---|---|---|---|---|
| | | | Het novel | Estimated false | false | |
| Variation | Total | | FDR | positives on | positives/ | Estimated |
| Type | detected | Novel | (Table S8) | genome | Mbp | FDR |
| SNP | 3,076,869 | 310,690 | 2-6% | 7k-17k | 2.3-6.1 | 0.2-0.6% |
| Deletion | 168,726 | 61,960 | 8-14% | 5k-8k | 1.8-3.0 | 3.0-5.0% |
| Insertion | 168,909 | 61,933 | 11-18% | 7k-11k | 2.3-3.9 | 3.9-6.5% |
| Block | 62,783 | 30,445 | 11-29% | 3k-9k | 1.1-3.1 | 5.2-13.9% |
| substitution | | | | | | |

The concordance of 1M Infinium SNPs with called variants for NA07022 was determined by percent of data sorted by variant quality score. The percent of discordant loci can be decreased by using variant quality score thresholds that filter the percent of the data indicated.

Aberrant mate-pair gaps may indicate the presence of length-altering structural variants and rearrangements with respect to the reference genome. A total of 2,126 clusters of such anomalous mate-pairs were identified in NA07022. PCR-based confirmation was performed of one such heterozygous 1,500-base deletion. More than half of the clusters were consistent in size with the addition or deletion of a single Alu repeat element.

Some applications of complete genome sequencing may benefit from maximal discovery rates, even at the cost of additional false-positives, while for other applications, a lower discovery rate and lower false-positive rate can be preferable. The variant quality score was used to tune call rate and accuracy. Additionally, novelty rate (relative to dbSNP) was also a function of variant quality score.

The proportion of variation calls that are novel (not corroborated by dbSNP, release 129) varied with variant quality score threshold. The variant quality score can be used to select the desired balance between novelty rate and call rate. We plotted the number of known and novel variations detected at a single variant quality score threshold. Note that novelty rate is not a direct proxy for error rate and that variant quality score has a different meaning for different variant types.

The NA07022 data were processed with Trait-o-Matic automated annotation software yielding 1,159 annotated variants, 14 of which have possible disease implications.

Once loci for confirmation sequencing were identified, PCR primer sequences flanking the variants of interest were designed with the JCVI Primer Designer (http://sourceforge.net/projects/primerdesigner/,S1), a management and pipeline suite build atop Primer3. Synthetic oligos [Integrated DNA Technologies, Inc. (IDT), Coralville, Iowa] were used to amplify the loci with Taq polymerase and the PCR products were purified by SPR1 (Agencourt). Purified PCR products were Sanger sequenced on both strands (MCLAB). The resulting traces were filtered for high quality data, run through TraceTuner (http://sourceforge.net/projects/tracetuner/) to generate mixed base calls, and aligned to their expected read sequence with applications from the EMBOSS Software Suite (http://emboss.sourceforge.net/,). For each locus, the expected read sequence was generated for each strand by modifying the reference based on the predicted variation(s) to reflect the combination of the two allele sequences. A locus was determined to be confirmed if the corresponding traces aligned exactly to the expected read sequence at that variant position for at least one strand. Any strand contradiction or discrepancies due to background noise were resolved by visual inspection of the traces.

Analysis of Coding SNPs

All SNP variants identified in NA07022 were analyzed with Trait-o-Matic software. This software, run as a website, returns all non-synonymous SNP (nsSNP) variants found in HGMD, OMIM and SNPedia (cited SNPs), as well as all nsSNPs not specifically listed in the preceding databases, but that occur in genes listed in OMIM (uncited nsSNPs). Analysis of the NA07022 genome with Trait-o-Matic returned 1,141 variants, including 605 cited nsSNPs, and 536 uncited nsSNPs. Filtering of 320 variants with BLOSUM100 scores below 3 and 725 variants with a minor allele frequency (MAF)>0.06 in the Caucasian/European (CEU) population (weighted average of HapMap and 1000 genomes frequency data) left 55 cited nsSNPs and 41 uncited SNPs. Forty-one cited nsSNPs were removed either because their phenotypic evidence was based solely on association studies, or because they were not disease-associated (e.g. olfactory receptor, blood type, eye color), and 38 uncited nsSNPs were removed because they had non-obvious functional consequences.

### Example 4

### Wash Step Before Anchor Hybridization

### Pre-Anchor Wash: Inside Positions

DNBs preps were loaded into flow slide lanes as described above.

A wash step was included before anchor hybridization on inside positions. The pre-anchor wash reagent (PAW) was either 0.1 mM CTAB or 10 mM citric acid for ten minutes after addition of the pre-post strip (PPS) reagent (0.1% Tween) and prior to anchor hybridization for inside positions.

The results are shown in FIG. 5. Discordance for the inside positions decreased and mapped bases increased in those lanes receiving a CTAB or citric acid wash. Apparent discordance for outside positions increased, most likely due to the decrease in discordance of inside positions. All outside positions received the standard procedure with no lane variables. Citric acid provided a slightly higher improvement in discordance and mapping yield than was observed with CTAB.

In separate studies it was found that a citric acid wash for 4 minutes produced similar improvements in discordance and mappable yield as 10 minutes.

### Pre-Anchor Wash: Outside Positions

Various treatments were tested in order to reduce the decay of quality of data from sequencing reactions over 70 cycles, which was observed beginning around cycle 30 to 40. In the standard sequencing protocol, the inside positions are sequenced after the outside positions. As used herein with reference to “double cPAL,” the term “inside positions” refers to the five bases immediately adjacent an adaptor; therefore, the inside positions can be sequenced using an anchor and a probe. The term “outside positions” refers to the next five bases, which can be sequenced using an anchor, a degenerate anchor (which permits sequencing to be performed farther out from the adaptor), and a probe.

Polyethylene glycol (PEG) concentration in the probe mix was increased in order to use the volume exclusion properties of PEG to increase the effective concentration of the probe. Although PEG did not have the desired effect in general, one batch of PEG did improve data quality. Upon further testing, it was determined that this batch had a low pH. We tested other reagents that generate a positive charge. Polyamines (spermine and spermidine) and polylysine did not improve data quality under the conditions that were tested. Cationic surfactants (e.g., cetyltrimethylammonium bromide or CTAB) did improve data quality, while neutral (e.g., Tween or Tritonics 100) or anionic surfactants (e.g., SDS) had no effect. Weak acids (e.g., citric acid) also improved data quality.

The wash step consisted of two lane loadings for a total time of five minutes. Pre-post strip (PPS) reagent (0.1% Tween) or pre-anchor wash (PAW) reagent (10 mM citric acid; 2 ml/well) was added to the wells of a standard sequencing plate and dispensed onto the slide for five minutes after addition of the PPS reagent and prior to anchor ligation. Standard cPAL sequencing reactions were performed, and the average discordance was determined for all positions and lanes that received the treatment.

We observed an improvement in both discordance (median: PPS=3.38%, PAW=2.86%) and mapping yield (fully mapped percentage; median: PPS=50.3, PAW=51.2) with the use of citric acid as a pre-anchor wash.

The present specification provides a complete description of the methodologies, systems and/or structures and uses thereof in example aspects of the presently-described technology. Although various aspects of this technology have been described above with a certain degree of particularity, or with reference to one or more individual aspects, those skilled in the art could make numerous alterations to the disclosed aspects without departing from the spirit or scope of the technology hereof. Since many aspects can be made without departing from the spirit and scope of the presently described technology, the appropriate scope resides in the claims hereinafter appended. Other aspects are therefore contemplated. Furthermore, it should be understood that any operations may be performed in any order, unless explicitly claimed otherwise or a specific order is inherently necessitated by the claim language. It is intended that all matter contained in the above description and shown in the accompanying drawings shall be interpreted as illustrative only of particular aspects and are not limiting to the embodiments shown. Unless otherwise clear from the context or expressly stated, any concentration values provided herein are generally given in terms of admixture values or percentages without regard to any conversion that occurs upon or following addition of the particular component of the mixture. To the extent not already expressly incorporated herein, all published references and patent documents referred to in this disclosure are incorporated herein by reference in their entirety for all purposes. Changes in detail or structure may be made without departing from the basic elements of the present technology as defined in the following claims.

What is claimed:

1. A method of sequencing a target sequence of a nucleic acid molecule, the method comprising:
(a) providing a surface comprising the nucleic acid molecule, the nucleic acid molecule comprising: (i) a first adaptor comprising a first anchor site; and (ii) the target sequence;
(b) applying to the surface an aqueous wash solution comprising an effective amount of an acid, a cationic surfactant, or both an acid and a cationic surfactant;
(c) hybridizing an anchor to the first anchor site;
(d) extending the anchor to produce an anchor extension product;
(e) detecting the extension product, thereby identifying a base of the target sequence; and
(f) repeating steps (b) to (e) until the sequence of the target sequence is determined.

2. The method of claim 1, wherein the surface comprising the nucleic acid molecule is an nucleic acid array comprising a surface and a plurality of the nucleic acid molecules attached to the surface.

3. The method of claim 1, wherein the nucleic acid molecule is a concatemer comprising a plurality of monomer units, each monomer unit comprising the first adaptor and the target sequence.

4. The method of claim 1 comprising extending the anchor by adding a nucleotide to the anchor or a product of a previous extension of the anchor.

5. The method of claim 1 comprising extending the anchor by ligating a sequencing probe to the anchor or a product of a previous extension of the anchor.

6. The method of claim 5, comprising extending the anchor by: (i) ligating one or more extension anchors to the anchor, and (ii) ligating the sequence probe to said one or more extension anchors.

7. The method of claim 5, comprising stripping the extension product from the nucleic acid molecule before repeating steps (b) to (e).

8. The method of claim 1 wherein the aqueous wash solution comprises citric acid.

9. The method of claim 1 wherein the aqueous wash solution comprises cetyltrimethylammonium bromide (CTAB).

10. The method of claim 1 wherein the aqueous wash solution comprises an amount of a weak acid or a cationic surfactant that is effective to reduce discordance by 5 percent or more or to increase a mappable yield by 0.5 percent or more or both compared with a suitable control.

11. The method of claim 1 comprising applying to the surface an aqueous wash solution before hybridizing the anchor to the first anchor site.

12. An aqueous wash solution configured for sequencing a nucleic acid molecule that is attached to a surface, the wash solution comprising an acid, a cationic surfactant, or both, wherein the wash solution is effective to detectably reduce discordance or to increase a mappable yield by 0.5 percent or more or both compared with a suitable control.

13. The wash solution of claim 12 wherein the wash solution is effective to reduce discordance by 5 percent or more compared to a suitable control.

14. The wash solution of claim 12 wherein the wash solution is effective to increase a mappable yield by 0.5 percent or more compared to a suitable control.

15. The method of claim 1, wherein the aqueous was solution applied in step (b) is a wash solution according to claim 12.
